(19) United States
(12) Patent Application Publication
(10) Pub. No.: US 2015/0105350 A1
(43) Pub. Date: Apr. 16, 2015

(54) COMBINATION THERAPY COMPRISING TENOFOVIR ALAFENAMIDE HEMIFUMARATE AND COBICISTAT FOR USE IN THE TREATMENT OF VIRAL INFECTIONS

(71) Applicant: Gilead Sciences, Inc., Foster City, CA (US)

(72) Inventor: Srinivasan Ramanathan, San Carlos, CA (US)

(73) Assignee: Gilead Sciences, Inc., Foster City, CA (US)

(21) Appl. No.: 14/376,116

(22) PCT Filed: Feb. 1, 2013
(86) PCT No.: PCT/US13/24438

§ 371 Date: Dec. 12, 2014

Related U.S. Application Data
(60) Provisional application No. 61/594,894, filed on Feb. 3, 2012.
(60) Provisional application No. 61/618,411, filed on Mar. 30, 2012.
(60) Provisional application No. 61/624,676, filed on Apr. 16, 2012.
(60) Provisional application No. 61/692,392, filed on Aug. 23, 2012.
(60) Provisional application No. 61/737,493, filed on Dec. 14, 2012.

(51) Int. Cl. A61K 31/675; A61K 31/513; A61K 31/47; A61K 31/5377

(52) U.S. Cl. CPC A61K 31/675; A61K 31/5377; A61K 31/513; A61K 31/47
USPC 514/81

(57) ABSTRACT

The use of the hemifumarate form of {9-[(R)-2-[[(S)-[[(S)-1-(isopropoxycarbonyl)ethyl]amino]phenoxyphosphinyl]methoxy]propyl]adenine} (tenofovir alafenamide hemifumarate) in combination with cobicistat is disclosed. In addition, the combination of tenofovir alafenamide hemifumarate, cobicistat, emtricitabine, and elvitegravir, and the combination of tenofovir alafenamide hemifumarate, cobicistat, emtricitabine, and darunavir, are disclosed.

### CROSS-REFERENCE TO RELATED APPLICATIONS

This application claims the benefit of priority from U.S. Provisional Patent Application No. 61/594,894, filed Feb. 3, 2012; U.S. Provisional Patent Application No. 61/618,411, filed Mar. 30, 2012; U.S. Provisional Patent Application No. 61/624,676, filed Apr. 16, 2012; U.S. Provisional Patent Application No. 61/692,392, filed Aug. 23, 2012; and U.S. Provisional Patent Application No. 61/737,493, filed Dec. 14, 2012, the content of each of which is hereby incorporated by reference herein in its entirety.

### BACKGROUND OF THE INVENTION

### Field of the Invention

Tenofovir {9-R-[(2-phosphonomethoxy)propyl]adenine}, an acyclic nucleotide analog of dAMP, is a potent in vitro and in vivo inhibitor of human immunodeficiency virus type 1 (HIV-1) replication. Tenofovir is sequentially phosphorylated in the cell by AMP kinase and nucleoside diphosphate kinase to the active species, tenofovir diphosphate, which acts as a competitive inhibitor of HIV-1 reverse transcriptase that terminates the growing viral DNA chain. The presence of a nonhydrolyzable phosphonic acid moiety in tenofovir circumvents an initial phosphorylation step that can be rate limiting for the activation of nucleoside analog inhibitors of HIV reverse transcriptase. Due to the presence of a phosphonate group, tenofovir is negatively charged at neutral pH, thus limiting its oral bioavailability.

Tenofovir disoproxil fumarate (TDF; VIREAD®), the first generation oral prodrug of tenofovir, has been extensively studied in clinical trials and has received marketing authorization in many countries as a once-daily tablet (300 mg) in combination with other antiretroviral agents for the treatment of HIV-1 infection.

U.S. Pat. No. 7,390,791 describes certain prodrugs of phosphonate nucleotide analogs that are useful in therapy. One such prodrug is 9-[(R)-2-[[(S)-[[(S)-1-(isopropoxycarbonyl)ethyl]amino]phenoxyphosphinyl]-methoxy]propyl]adenine 16:

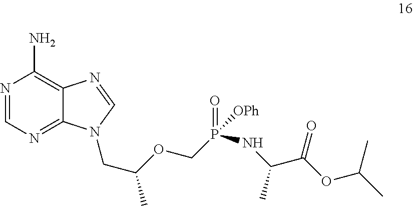 [SMILES: CC(C)OC(=O)[C@H](C)N[P@](=O)(CO[C@H](C)Cn1cnc2c(N)ncnc21)Oc1ccccc1]

GS-7340 {9-[(R)-2-[[(S)-[[(S)-1-(isopropoxycarbonyl)ethyl]amino]phenoxyphosphinyl]methoxy]propyl]adenine} is an isopropylalaninyl phenyl ester prodrug of tenofovir (9-[(2-phosphonomethoxy) propyl]adenine). GS-7340 exhibits potent anti-HIV activity 500- to 1000-fold enhanced activity relative to tenofovir against HIV-1 in T cells, activated peripheral blood mononuclear lymphocytes (PBMCs), and macrophages. GS-7340 also has enhanced ability to deliver and increase the accumulation of the parent tenofovir into PBMCs and other lymphatic tissues in vivo. It is also a potent inhibitor of hepatitis B virus.

GS-7340 is metabolized to tenofovir, which is not dependent on an intracellular nucleoside kinase activity for the first step in the conversion to the active metabolite, tenofovir diphosphate (PMPApp). The cellular enzymes responsible for tenofovir metabolism to the active diphosphorylated form are adenylate kinase and nucleotide diphosphate kinase, which are highly active and ubiquitous. Adenylate kinase exists as multiple isozymes (AK1 to AK4), with the phosphorylation of tenofovir mediated most efficiently by AK2.

Tenofovir does not interact significantly with human drug metabolizing cytochrome P450 enzymes or UDP-glucuronosyltransferases as a substrate, inhibitor, or inducer, in vitro or in vivo in humans. GS-7340 has limited potential to alter cytochrome P450 enzyme activity through inhibition (IC₅₀>7 μM compared to all isoforms tested). Similarly GS-7340 does not inhibit UGT1A1 function at concentrations up to 50 μM. In addition, GS-7340 is not an activator of either the aryl hydrocarbon receptor or human pregnane X receptor.

Although tenofovir and GS-7340 show desirable activities, the treatment cost and the potential for unwanted side effects can both increase as the required dose of a drug increases. Therefore, there is a need for methods and compositions that are useful for achieving an acceptable anti-viral effect using a reduced dose of tenofovir or GS-7340.

Along with U.S. Pat. No. 7,390,791, U.S. Pat. No. 7,803,788 (the content of each of which is incorporated by reference herein in its entirety) also describes certain prodrugs of phosphonate nucleotide analogs that are useful in therapy. As noted above, one such prodrug is 9-[(R)-2-[[(S)-[[(S)-1-(isopropoxycarbonyl)ethyl]amino]phenoxyphosphinyl]methoxy]propyl]adenine. This compound is also known by the Chemical Abstract name L-alanine, N—[(S)-[[(1R)-2-(6-amino-9H-purin-9-yl)-1-methylethoxy]methyl]phenoxyphosphinyl]-, 1-methylethyl ester. U.S. Pat. Nos. 7,390,791 and 7,803,788 disclose a monofumarate form of this compound and its preparation method (see, e.g., Example 4).

### SUMMARY OF THE INVENTION

It has been determined that the systemic exposure to GS-7340 in humans improves when GS-7340 is administered with cobicistat (1,3-thiazol-5-ylmethyl (2R,5R)-(5-{[(2S)-2-[(methyl{[2-(propan-2-yl)-1,3-thiazol-4-yl]methyl}carbamoyl)amino]]-4-(morpholin-4-yl)butanamido}-1,6-diphenylhexan-2-yl)carbamate). When administered with cobicistat, GS-7340 was calculated to have a systemic exposure equivalent 2.2 fold higher than a dose of GS-7340 alone. In another case, GS-7340 administered with cobicistat was calculated to have a systemic exposure equivalent 3-4 fold higher than a dose of GS-7340 alone. In another case, GS-7340 administered with cobicistat was calculated to have a systemic exposure equivalent 1.3 fold higher than a dose of GS-7340 alone.

In one embodiment, the invention provides for the use of the compound GS-7340 or a pharmaceutically acceptable salt thereof and cobicistat, or a pharmaceutically acceptable salt thereof, for the prophylactic or therapeutic treatment of a viral infection in a human. The cobicistat may be coadministered with GS-7340. GS-7340 or a pharmaceutically acceptable salt thereof, may be used in amounts of 3 mg, 8±3 mg, 10±5 mg, 25±5 mg, or 40±10 mg, or other ranges as set forth below. Cobicistat or a pharmaceutically acceptable salt thereof may be used in an amount of 50-500 mg, 100-400 mg, 100-300 mg or 150 mg. GS-7340, or a pharmaceutically acceptable salt thereof, and cobicistat or a pharmaceutically acceptable salt thereof, may be coadministered. A unit dosage form comprising a daily amount of GS-7340 or a pharmaceutically acceptable salt thereof, and a daily amount of cobicistat or pharmaceutically acceptable salt thereof, may be used. The virus of the viral infection may be human immunodeficiency virus (HIV) or Hepatitis B virus (HBV).

In one embodiment, the invention provides for the use of the compound GS-7340, or a pharmaceutically acceptable salt thereof, and cobicistat, or a pharmaceutically acceptable salt thereof, for improving the pharmacokinetics of GS-7340. The cobicistat may be coadministered with GS-7340. GS-7340, or a pharmaceutically acceptable salt thereof, may be used in amounts of 3 mg, 8±3 mg, 10±5 mg, 25±5 mg, or 40±10 mg, or other ranges as set forth below. Cobicistat or a pharmaceutically acceptable salt thereof may be used in an amount of 50-500 mg, 100-400 mg, 100-300 mg or 150 mg. GS-7340, or a pharmaceutically acceptable salt thereof, and the cobicistat or pharmaceutically acceptable salt thereof may be coadministered. A unit dosage form comprising a daily amount GS-7340 or a pharmaceutically acceptable salt thereof, and a daily amount cobicistat or pharmaceutically acceptable salt thereof may be used. The use may be for the prophylactic or therapeutic treatment of a viral infection in a human. The virus may be human immunodeficiency virus (HIV) or Hepatitis B virus (HBV).

In one embodiment, the invention provides for the use of the compound GS-7340 or a pharmaceutically acceptable salt thereof and cobicistat, or a pharmaceutically acceptable salt thereof, for improving the C_{max of GS-}7340. The cobicistat may be coadministered with GS-7340. GS-7340 or a pharmaceutically acceptable salt thereof, may be used in amounts of 3 mg, 8±3 mg, 10±5 mg, 25±5 mg, or 40±10 mg or other ranges as set forth below. Cobicistat or a pharmaceutically acceptable salt thereof may be used in an amount of 50-500 mg, 100-400 mg, 100-300 mg or 150 mg. GS-7340, or a pharmaceutically acceptable salt thereof, and the cobicistat or pharmaceutically acceptable salt thereof may be coadministered. A unit dosage form comprising a daily amount of GS-7340 or a pharmaceutically acceptable salt thereof, and a daily amount of cobicistat or pharmaceutically acceptable salt thereof may be used. The use may be for the prophylactic or therapeutic treatment of a viral infection in a human. The virus may be human immunodeficiency virus (HIV) or Hepatitis B virus (HBV).

In one embodiment, the invention provides for the use of the compound GS-7340 or a pharmaceutically acceptable salt thereof and cobicistat, or a pharmaceutically acceptable salt thereof, for improving blood levels of GS-7340. The cobicistat may be coadministered with GS-7340. GS-7340 or a pharmaceutically acceptable salt thereof, may be used in amounts of 3 mg, 8±3 mg, 10±5 mg, 25±5 mg, or 40±10 mg or other ranges as set forth below. Cobicistat or a pharmaceutically acceptable salt thereof may be used in an amount of 50-500 mg, 100-400 mg, 100-300 mg or 150 mg. GS-7340, or a pharmaceutically acceptable salt thereof, and the cobicistat or pharmaceutically acceptable salt thereof may be coadministered. A unit dosage form comprising a daily amount GS-7340 or a pharmaceutically acceptable salt thereof, and a daily amount cobicistat or pharmaceutically acceptable salt thereof may be used. The use may be for the prophylactic or therapeutic treatment of a viral infection in a human. The virus may be human immunodeficiency virus (HIV) or Hepatitis B virus (HBV).

In one embodiment, the invention provides for a composition comprising a unit-dosage form of GS-7340 or a pharmaceutically acceptable salt thereof; a unit-dosage form of cobicistat, or a pharmaceutically acceptable salt thereof; and a pharmaceutically acceptable carrier or diluent. The composition may include GS-7340 or a pharmaceutically acceptable salt thereof in amounts of 3 mg, 8±3 mg, 10±5 mg, 25±5 mg, or 40±10 mg or other ranges as set forth below. The composition may include cobicistat in amounts of 50-500 mg, 100-400 mg, 100-300 mg or 150 mg. The unit dosage form may be a single daily dosage.

In one embodiment, the invention provides for a kit comprising: (1) GS-7340, or a pharmaceutically acceptable salt thereof; (2) cobicistat, or a pharmaceutically acceptable salt thereof; (3) one or more containers; and (4) prescribing information regarding administering the GS-7340 or a pharmaceutically acceptable salt thereof with the cobicistat or the pharmaceutically acceptable salt thereof. The kit may include GS-7340 or a pharmaceutically acceptable salt thereof in amounts of 3 mg, 8±3 mg, 10±5 mg, 25±5 mg, or 40±10 mg or other ranges as set forth below. The kit may include cobicistat in amounts of 50-500 mg, 100-400 mg, 100-300 mg or 150 mg.

In one embodiment, the invention provides for a method of treating a viral infection in a human comprising coadministering GS-7340 with cobicistat, or a pharmaceutically acceptable salt thereof, wherein the dose of cobicistat coadministered with the GS-7340 provides a systemic exposure of GS-7340 comparable to the systemic exposure obtainable by administration of a greater dose of GS-7340 in the absence of cobicistat. GS-7340 or a pharmaceutically acceptable salt thereof in amounts of 3 mg, 8±3 mg, 10±5 mg, 25±5 mg, or 40±10 mg or other ranges as set forth below may be coadministered with cobicistat. Cobicistat in amounts of 50-500 mg, 100-400 mg, 100-300 mg or 150 mg may be coadministered with GS-7340. The virus may be human immunodeficiency virus (HIV) or Hepatitis B virus (HBV).

In one embodiment, the invention provides for a method for inhibiting activity of a retroviral reverse transcriptase in a human comprising coadministering GS-7340 with cobicistat, or a pharmaceutically acceptable salt thereof, wherein the dose of cobicistat coadministered with the GS-7340 provides a systemic exposure of GS-7340 comparable to the systemic exposure obtainable by administration of a greater dose of GS-7340 in the absence of cobicistat. GS-7340 or a pharmaceutically acceptable salt thereof in amounts of 3 mg, 8±3 mg, 10±5 mg, 25±5 mg, or 40±10 mg or other ranges as set forth below may be coadministered with cobicistat. Cobicistat in amounts of 50-500 mg, 100-400 mg, 100-300 mg or 150 mg may be coadministered with GS-7340. The virus may be human immunodeficiency virus (HIV).

In one embodiment, the invention provides for the use of the compound GS-7340 or a pharmaceutically acceptable salt thereof coadministered with cobicistat, or a pharmaceutically acceptable salt thereof for the manufacture of a medicament for treating a viral infection. The invention further provides for the use of the compound GS-7340 or a pharmaceutically acceptable salt thereof coadministered with cobicistat, or a pharmaceutically acceptable salt thereof for the manufacture of a medicament for treating a viral infection in a human. GS-7340 or a pharmaceutically acceptable salt thereof may be used in a subtherapeutic amount (or, in some embodiments throughout, in a therapeutic amount). GS-7340 or a pharmaceutically acceptable salt thereof may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. Cobicistat may be used in an amount that provides a systemic exposure of GS-7340 comparable to the systemic exposure obtainable by administration of a greater dose of GS-7340 in the absence of cobicistat is used in the manufacture of the medicament. The virus may be human immunodeficiency virus (HIV) or Hepatitis B virus (HBV).

In one embodiment, the invention provides for the use of the compound GS-7340 or a pharmaceutically acceptable salt thereof coadministered with cobicistat, or a pharmaceutically acceptable salt thereof for the manufacture of a medicament for inhibiting activity of a retroviral reverse transcriptase. The invention further provides for the use of the compound GS-7340 or a pharmaceutically acceptable salt thereof coadministered with cobicistat, or a pharmaceutically acceptable salt thereof for the manufacture of a medicament for inhibiting activity of a retroviral reverse transcriptase in a human. GS-7340 or a pharmaceutically acceptable salt thereof may be used in a subtherapeutic amount. GS-7340 or a pharmaceutically acceptable salt thereof may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. The cobicistat may be used in an amount that provides a systemic exposure of GS-7340 comparable to the systemic exposure obtainable by administration of a greater dose of GS-7340 in the absence of cobicistat is used in the manufacture of the medicament. The virus may be human immunodeficiency virus (HIV).

In one embodiment, the invention provides for the use of cobicistat, or a pharmaceutically acceptable salt thereof, to prepare a medicament useful for improving the pharmacokinetics of GS-7340, or a pharmaceutically acceptable salt thereof, following administration to a human. GS-7340 or a pharmaceutically acceptable salt thereof may be used in a subtherapeutic amount. GS-7340 or a pharmaceutically acceptable salt thereof may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. The cobicistat may be used in an amount that provides a systemic exposure of GS-7340 comparable to the systemic exposure obtainable by administration of a greater dose of GS-7340 in the absence of cobicistat is used in the manufacture of the medicament. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof, to prepare a medicament useful for improving the pharmacokinetics of {9-[(R)-2-[[(S)-[[(S)-1-(isopropoxycarbonyl)ethyl]amino]phenoxyphosphinyl]methoxy]propyl]adenine}, or a pharmaceutically acceptable salt thereof, following administration to a human. GS-7340 or a pharmaceutically acceptable salt thereof may be used in a subtherapeutic amount. {9-[(R)-2-[[(S)-[[(S)-1-(isopropoxycarbonyl)ethyl]amino]phenoxyphosphinyl]methoxy]propyl]adenine}, or a pharmaceutically acceptable salt thereof, may be used in amounts of 3 mg, 8±3 mg, 10±5 mg, 25±5 mg, or 40±10 mg or other ranges as set forth herein. Cobicistat may be used in amounts of 50-500 mg, 100-400 mg, 100-300 mg or 150 mg. The cobicistat may be used in an amount that provides a systemic exposure of GS-7340 comparable to the systemic exposure obtainable by administration of a greater dose of GS-7340 in the absence of cobicistat is used in the manufacture of the medicament. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of GS-7340 by about 30-70%, or a pharmaceutically acceptable salt thereof, upon administration of the cobicistat. The use may be for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of GS-7340 by about 2-4 fold, or a pharmaceutically acceptable salt thereof, upon administration of the cobicistat. In one embodiment, the invention provides for the use of cobicistat, or a pharmaceutically acceptable salt thereof; to prepare a medicament for a human useful for reducing a dose of GS-7340 by about 3 fold, or a pharmaceutically acceptable salt thereof, upon administration of the cobicistat. The use may be for the prophylactic or therapeutic treatment of a viral infection in a human. The virus may be human immunodeficiency virus (HIV) or Hepatitis B virus (HBV).

In one embodiment, the invention provides for a method of treating a viral infection in a human comprising coadministering 1) GS-7340 or a pharmaceutically acceptable salt thereof; and 2) cobicistat, or a pharmaceutically acceptable salt thereof to the human. GS-7340 or a pharmaceutically acceptable salt thereof is administered in a subtherapeutic amount. The virus may be human immunodeficiency virus (HIV) or Hepatitis B virus (HBV).

In one embodiment, the invention provides for a use of a subtherapeutic dose of GS-7340 coadministered with cobicistat for treating a viral infection. The virus may be human immunodeficiency virus (HIV) or Hepatitis B virus (HBV).

In one embodiment, the invention provides for the use of a subtherapeutic dose of GS-7340 coadministered with cobicistat for inhibiting retroviral reverse transcriptase. The virus may be human immunodeficiency virus (HIV).

In one embodiment, the invention provides for an anti-virus agent(s) comprising (a) a compound GS-7340 or a pharmaceutically acceptable salt thereof and (b) cobicistat, or a pharmaceutically acceptable salt thereof. The anti-virus agent(s) may include GS-7340 or a pharmaceutically acceptable salt thereof may be used in amounts of 3 mg, 8±3 mg, 10±5 mg, 25±5 mg, or 40±10 mg or other ranges as set forth below. The anti-virus agent(s) may include cobicistat in amounts of 50-500 mg, 100-400 mg, 100-300 mg or 150 mg. The cobicistat may be used in an amount that provides a systemic exposure of GS-7340 comparable to the systemic exposure obtainable by administration of a greater dose of GS-7340 in the absence of cobicistat is used in the manufacture of the medicament. The anti-virus agent may further include 200 mg of emtricitabine and 150 mg of elvitegravir. The anti-virus agent may further include 150 mg cobicistat, 8 or less mg GS-7340, 150 mg elvitegravir, and 200 mg emtricitabine. The anti-virus agent may further include 150 mg cobicistat, 25 or less mg GS-7340, 150 mg elvitegravir, and 200 mg emtricitabine. The anti-virus agent may further include 150 mg cobicistat, 10 or less mg GS-7340, 150 mg elvitegravir, and 200 mg emtricitabine. The anti-virus agent may include 150 mg cobicistat, 8 mg GS-7340, 150 mg elvitegravir, and 200 mg emtricitabine. The anti-virus agent may include 150 mg cobicistat, 10 mg GS-7340, 150 mg elvitegravir, and 200 mg emtricitabine.

In one embodiment, the invention provides for a unit-dosage of GS-7340 or a pharmaceutically acceptable salt thereof and cobicistat, or a pharmaceutically acceptable salt thereof, wherein the unit-dosage is a daily dose. GS-7340 may be present in a subtherapeutic amount. The unit-dosage may further include 150 mg cobicistat, 8 or less mg GS-7340, 150 mg elvitegravir, and 200 mg emtricitabine. The unit-dosage may further include 150 mg cobicistat, 25 or less mg GS-7340, 150 mg elvitegravir, and 200 mg emtricitabine. The unit-dosage may further include 150 mg cobicistat, 10 or less mg GS-7340, 150 mg elvitegravir, and 200 mg emtricitabine. The unit-dosage may include 150 mg cobicistat, 10 mg GS-7340, 150 mg elvitegravir, and 200 mg emtricitabine.

In one embodiment, the invention provides the use of cobicistat, or a pharmaceutically acceptable salt thereof, to prepare a medicament useful for improving the pharmacokinetics of {9-[(R)-2-[[(S)-[[(S)-1-(isopropoxycarbonyl)ethyl]amino]phenoxyphosphinyl]methoxy]propyl]adenine}, or a pharmaceutically acceptable salt thereof, following administration to a human. The medicament may be used for the prophylactic or therapeutic treatment of a viral infection in a human. The virus may be, e.g., human immunodeficiency virus (HIV) or Hepatitis B virus (HBV).

In one embodiment, the invention provides cobicistat for use in improving the pharmacokinetics of {9-[(R)-2-[[(S)-[[(S)-1-(isopropoxycarbonyl)ethyl]amino]phenoxyphosphinyl]methoxy]propyl]adenine} or a pharmaceutically acceptable salt thereof, following administration to a human. The use may be for the prophylactic or therapeutic treatment of a viral infection in a human. The virus may be human immunodeficiency virus (HIV) or Hepatitis B virus (HBV).

In one embodiment, the invention provides a kit comprising: (1) {9-[(R)-2-[[(S)-[[(S)-1-(isopropoxycarbonyl)ethyl]amino]phenoxyphosphinyl]methoxy]propyl]adenine}, or a pharmaceutically acceptable salt thereof; (2) cobicistat, or a pharmaceutically acceptable salt thereof; (3) one or more containers; and (4) prescribing information regarding administering the {9-[(R)-2-[[(S)-[[(S)-1-(isopropoxycarbonyl)ethyl]amino]phenoxyphosphinyl]methoxy]propyl]adenine} or a pharmaceutically acceptable salt thereof with the cobicistat or a pharmaceutically acceptable salt thereof.

In one embodiment, the invention provides a kit comprising: (1) a unit dosage form comprising 5-100 mg of {9-[(R)-2-[[(S)-[[(S)-1-(isopropoxycarbonyl)ethyl]amino]phenoxyphosphinyl]methoxy]propyl]adenine}, or a pharmaceutically acceptable salt thereof; (2) a unit dosage form comprising 150 mg cobicistat, or a pharmaceutically acceptable salt thereof; (3) one or more containers; and (4) prescribing information regarding administering the {9-[(R)-2-[[(S)-[[(S)-1-(isopropoxycarbonyl)ethyl]amino]phenoxyphosphinyl]methoxy]propyl]adenine} or a pharmaceutically acceptable salt thereof with cobicistat or a pharmaceutically acceptable salt thereof.

In one embodiment, the invention provides a use of {9-[(R)-2-[[(S)-[[(S)-1-(isopropoxycarbonyl)ethyl]amino]phenoxyphosphinyl]methoxy]propyl]adenine} or its pharmaceutically acceptable salt for the manufacture of a medicament for inhibiting activity of a retroviral reverse transcriptase in a human, comprising administering GS-7340 or a pharmaceutically acceptable salt thereof, and cobicistat, or a pharmaceutically acceptable salt thereof to the human. The virus may be human immunodeficiency virus (HIV).

In one embodiment, the invention provides {9-[(R)-2-[[(S)-[[(S)-1-(isopropoxycarbonyl)ethyl]amino]phenoxyphosphinyl]methoxy]propyl]adenine} or its pharmaceutically acceptable salt; and cobicistat, or a pharmaceutically acceptable salt thereof; for use in inhibiting activity of a retroviral reverse transcriptase in a human.

In one embodiment, the invention provides a use of cobicistat, or a pharmaceutically acceptable salt thereof, to prepare a medicament for a human useful for reducing a dose between about 30-70% of {9-[(R)-2-[[(S)-[[(S)-1-(isopropoxycarbonyl)ethyl]amino]phenoxyphosphinyl]methoxy]propyl]adenine} or a pharmaceutically acceptable salt thereof, upon administration of the cobicistat. The medicament may be used for the prophylactic or therapeutic treatment of a viral infection in a human. The virus may be human immunodeficiency virus (HIV) or Hepatitis B virus (HBV).

In one embodiment, the invention provides the use of {9-[(R)-2-[[(S)-[[(S)-1-(isopropoxycarbonyl)ethyl]amino]phenoxyphosphinyl]methoxy]propyl]adenine} or a pharmaceutically acceptable salt thereof; and cobicistat or a pharmaceutically acceptable salt thereof for the prophylactic or therapeutic treatment of a viral infection in a human. The use may be for the prophylactic or therapeutic treatment of a viral infection in a human. The virus may be human immunodeficiency virus (HIV) or Hepatitis B virus (HBV).

In one embodiment, the invention provides an anti-viral agent(s) comprising (a) {9-[(R)-2-[[(S)-[[(S)-1-(isopropoxycarbonyl)ethyl]amino]phenoxyphosphinyl]methoxy]propyl]adenine} or a pharmaceutically acceptable salt thereof, which is used in combination with (b) cobicistat or a pharmaceutically acceptable salt thereof for use in the prophylactic or therapeutic treatment of a viral infection in a human.

It has also been determined that the systemic exposure to tenofovir in humans improves when tenofovir is administered with cobicistat. When administered with cobicistat, tenofovir was calculated to have a systemic exposure equivalent 3 to 4 fold higher than a dose of tenofovir alone.

In one embodiment, the invention provides for the use of the compound tenofovir or a pharmaceutically acceptable salt thereof and cobicistat, or a pharmaceutically acceptable salt thereof, for the prophylactic or therapeutic treatment of a viral infection in a human. Tenofovir may be used in amounts of less than 300 mg, 200 mg or less and 100 mg or less. Cobicistat may be used in amounts of 50-500 mg, 100-400 mg, 100-300 mg, and 150 mg. The tenofovir or a pharmaceutically acceptable salt thereof, and the cobicistat or pharmaceutically acceptable salt thereof may be coadministered. The use may provide a unit dosage form comprising a daily amount tenofovir or a pharmaceutically acceptable salt thereof, and a daily amount cobicistat or pharmaceutically acceptable salt thereof is administered. The virus may be human immunodeficiency virus (HIV).

In one embodiment, the invention provides for a composition comprising a unit-dosage form of tenofovir or a pharmaceutically acceptable salt thereof; a unit-dosage form of cobicistat, or a pharmaceutically acceptable salt thereof; and a pharmaceutically acceptable carrier or diluent. Tenofovir may be present in the composition in amounts of less than 300 mg, 200 mg or less and 100 mg or less. Cobicistat may be used in amounts of 50-500 mg, 100-400 mg, 100-300 mg, and 150 mg.

In one embodiment, the invention provides for a kit that includes (1) tenofovir, or a pharmaceutically acceptable salt thereof; (2) cobicistat, or a pharmaceutically acceptable salt thereof; (3) one or more containers; and (4) prescribing information regarding administering the tenofovir or a pharmaceutically acceptable salt thereof with the cobicistat or the pharmaceutically acceptable salt thereof. Tenofovir may be present in the kit in amounts of less than 300 mg, 200 mg or less and 100 mg or less. Cobicistat may be used in amounts of 50-500 mg, 100-400 mg, 100-300 mg, and 150 mg.

In one embodiment, the invention provides for a method of treating a viral infection in a human that includes coadministering tenofovir with cobicistat, or a pharmaceutically acceptable salt thereof, wherein the dose of cobicistat coadministered with the tenofovir provides a systemic exposure of tenofovir comparable to the systemic exposure obtainable by administration of a greater dose of tenofovir in the absence of cobicistat. Tenofovir may be administered in amounts of less than 300 mg, 200 mg or less and 100 mg or less. Cobicistat may be administered in amounts of 50-500 mg, 100-400 mg, 100-300 mg, and 150 mg. The virus may be human immunodeficiency virus (HIV) or Hepatitis B virus (HBV).

In one embodiment, the invention provides for a method for inhibiting activity of a retroviral reverse transcriptase in a human comprising coadministering tenofovir with cobicistat, or a pharmaceutically acceptable salt thereof, wherein the dose of tenofovir coadministered with the cobicistat provides a systemic exposure of tenofovir comparable to the systemic exposure obtainable by administration of a greater dose of tenofovir in the absence of cobicistat. Tenofovir may be coadministered in amounts of less than 300 mg, 200 mg or less and 100 mg or less. Cobicistat may be coadministered in amounts of 50-500 mg, 100-400 mg, 100-300 mg, and 150 mg. The virus may be human immunodeficiency virus (HIV)

In one embodiment, the invention provides for the use of the compound tenofovir or a pharmaceutically acceptable salt thereof coadministered with cobicistat, or a pharmaceutically acceptable salt thereof for the manufacture of a medicament for treating a viral infection. The medicament may be used for the prophylactic or therapeutic treatment of a viral infection in a human. The virus may be human immunodeficiency virus (HIV) or Hepatitis B virus (HBV).

In one embodiment, the invention provides for the use of the compound tenofovir or a pharmaceutically acceptable salt thereof coadministered with cobicistat, or a pharmaceutically acceptable salt thereof for the manufacture of a medicament for treating a viral infection in a human. The tenofovir or a pharmaceutically acceptable salt thereof may be used in a subtherapeutic amount (or, in some embodiments throughout, in a therapeutic amount). Tenofovir may be administered in amounts of less than 300 mg, 200 mg or less and 100 mg or less. The cobicistat may be administered in an amount that provides a systemic exposure of tenofovir comparable to the systemic exposure obtainable by administration of a greater dose of tenofovir in the absence of cobicistat is used in the manufacture of the medicament. Cobicistat in an amount of 150 mg may be used in the manufacture of the medicament. The medicament may be used for the prophylactic or therapeutic treatment of a viral infection in a human. The virus may be human immunodeficiency virus (HIV) or Hepatitis B virus (HBV).

In one embodiment, the invention provides for the use of the compound tenofovir or a pharmaceutically acceptable salt thereof coadministered with cobicistat, or a pharmaceutically acceptable salt thereof for the manufacture of a medicament for inhibiting activity of a retroviral reverse transcriptase. The medicament may be used for the prophylactic or therapeutic treatment of a viral infection in a human. The virus may be human immunodeficiency virus (HIV) or Hepatitis B virus (HBV).

In one embodiment, the invention provides for use of the compound tenofovir or a pharmaceutically acceptable salt thereof coadministered with cobicistat, or a pharmaceutically acceptable salt thereof for the manufacture of a medicament for inhibiting activity of a retroviral reverse transcriptase in a human. The tenofovir or a pharmaceutically acceptable salt thereof may be used in a subtherapeutic amount. Tenofovir may be used in amounts of less than 300 mg, 200 mg or less and 100 mg or less. The cobicistat may be coadministered in an amount that provides a systemic exposure of tenofovir comparable to the systemic exposure obtainable by administration of a greater dose of tenofovir in the absence of cobicistat is used in the manufacture of the medicament. Cobicistat in an amount of 150 mg may be coadministered. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament useful for improving the pharmacokinetics of tenofovir, or a pharmaceutically acceptable salt thereof, following administration to a human. The tenofovir or a pharmaceutically acceptable salt thereof may be used in a subtherapeutic amount. Tenofovir or a pharmaceutically acceptable salt thereof, may be coadministered to the human in an amount of 100 mg or less, 200 mg or less or in amount less than 300 mg. Cobicistat may be used in an amount that provides a systemic exposure of tenofovir comparable to the systemic exposure obtainable by administration of a greater dose of tenofovir in the absence of cobicistat is used in the manufacture of the medicament. Cobicistat in an amount 150 mg may be used to prepare the medicament. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of tenofovir by about 30-70%, or a pharmaceutically acceptable salt thereof, upon administration of the cobicistat. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of tenofovir by about 2 to 4 fold, or a pharmaceutically acceptable salt thereof, upon administration of the cobicistat. In one embodiment, the invention provides for the use of cobicistat, or a pharmaceutically acceptable salt thereof; to prepare a medicament for a human useful for reducing a dose of tenofovir by about 3-fold, or a pharmaceutically acceptable salt thereof, upon administration of the cobicistat. The medicament may be used for the prophylactic or therapeutic treatment of a viral infection in a human. The virus may be human immunodeficiency virus (HIV) or Hepatitis B virus (HBV).

In one embodiment, the invention provides for a method of treating a viral infection in a human comprising coadministering 1) tenofovir or a pharmaceutically acceptable salt thereof; and 2) cobicistat, or a pharmaceutically acceptable salt thereof to the human. The tenofovir or a pharmaceutically acceptable salt thereof may be administered in a subtherapeutic amount. The virus may be human immunodeficiency virus (HIV) or Hepatitis B virus (HBV).

In one embodiment, the invention provides for a use of a subtherapeutic dose of tenofovir coadministered with cobicistat for treating a viral infection. The virus may be human immunodeficiency virus (HIV) or Hepatitis B virus (HBV).

In one embodiment, the invention provides for a use of a subtherapeutic dose of tenofovir coadministered with cobicistat for inhibiting retroviral reverse transcriptase. The virus may be human immunodeficiency virus (HIV).

In one embodiment, the invention provides for an anti-virus agent(s) comprising (a) a compound tenofovir or a pharmaceutically acceptable salt thereof and (b) cobicistat, or a pharmaceutically acceptable salt thereof. The tenofovir may be present in the anti-virus agent(s) in a subtherapeutic amount. The tenofovir may be present in the anti-virus agent(s) in an amount of 100 mg or less, 200 mg or less or less than 300 mg. The cobicistat coadministered with the tenofovir may be present in the anti-virus agent(s) in an amount that provides a systemic exposure of tenofovir comparable to the systemic exposure obtainable by administration of a greater dose of tenofovir in the absence of cobicistat. The anti-virus agent may further include cobicistat in an amount of 150 mg. The anti-virus agent may further include 200 mg of emtricitabine and 150 mg of elvitegravir. The anti-virus agent may include 150 mg cobicistat, 100 or less mg tenofovir, 150 mg elvitegravir, and 200 mg emtricitabine. The anti-virus agent may include 150 mg cobicistat, 200 or less mg tenofovir, 150 mg elvitegravir, and 200 mg emtricitabine. The anti-virus agent may include 150 mg cobicistat, less than 300 mg tenofovir, 150 mg elvitegravir, and 200 mg emtricitabine. The anti-virus agent may include 150 mg cobicistat, 50 mg tenofovir, 150 mg elvitegravir, and 200 mg emtricitabine. The virus may be human immunodeficiency virus (HIV) or Hepatitis B virus (HBV).

In one embodiment, the invention provides for a unit-dosage of tenofovir or a pharmaceutically acceptable salt thereof and cobicistat, or a pharmaceutically acceptable salt thereof, wherein the unit-dosage is a daily dose. Tenofovir may be present in a subtherapeutic amount. The unit-dosage may include 100 mg or less, 200 mg or less or less than 300 mg of tenofovir. The unit-dosage may include an amount of cobicistat that provides a systemic exposure of tenofovir comparable to the systemic exposure obtainable by administration of a greater dose of tenofovir in the absence of cobicistat. The unit-dosage may include 150 mg of cobicistat. The virus may be human immunodeficiency virus (HIV) or Hepatitis B virus (HBV).

Also described is a hemifumarate form of 9-[(R)-2-[[(S)-[[(S)-1-(isopropoxycarbonyl)ethyl]amino]phenoxyphosphinyl]methoxy]propyl]adenine. The name for 9-[(R)-2-[[(S)-[[(S)-1-(isopropoxycarbonyl)ethyl]amino]phenoxyphosphinyl]methoxy]propyl]adenine (GS-7340) is tenofovir alafenamide. The hemifumarate form of tenofovir alafenamide is also referred to herein as tenofovir alafenamide hemifumarate.

In one embodiment of the invention is provided tenofovir alafenamide hemifumarate, especially in combination with cobicistat and/or with other an additional therapeutic agent or agents.

In another embodiment is provided tenofovir alafenamide hemifumarate, wherein the ratio of fumaric acid to tenofovir alafenamide is 0.5±0.1, or 0.5±0.05, or 0.5±0.01, or about 0.5.

In one embodiment is provided tenofovir alafenamide hemifumarate in a solid form.

In one embodiment is provided tenofovir alafenamide hemifumarate that has an X-ray powder diffraction (XRPD) pattern having 2theta values of 6.9±0.2° and 8.6±0.2°. In another embodiment is provided tenofovir alafenamide hemifumarate wherein the XRPD pattern comprises 2theta values of 6.9±0.2°, 8.6±0.2°, 11.0±0.2°, 15.9±0.2°, and 20.2±0.2°.

In one embodiment is provided tenofovir alafenamide hemifumarate that has a differential scanning calorimetry (DSC) onset endotherm of 131±2° C., or 131±1° C.

In one embodiment is provided a pharmaceutical composition comprising tenofovir alafenamide hemifumarate and a pharmaceutically acceptable excipient. In another embodiment is provided the pharmaceutical composition, further comprising an additional therapeutic agent. In a further embodiment, the additional therapeutic agent is selected from the group consisting of human immunodeficiency virus (HIV) protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, CCR5 inhibitors, and additional protease inhibiting compounds.

In one embodiment is provided a method for treating a human immunodeficiency virus (HIV) infection comprising administering to a subject in need thereof a therapeutically effective amount of tenofovir alafenamide hemifumarate. In another embodiment is provided a method for treating an HIV infection comprising administering to a subject in need thereof a therapeutically effective amount of a pharmaceutical composition comprising tenofovir alafenamide hemifumarate. In a further embodiment, the method comprises administering to the subject one or more additional therapeutic agents selected from the group consisting of HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, CCR5 inhibitors, and additional protease inhibiting compounds.

In one embodiment is provided a method for treating a hepatitis B virus (HBV) infection comprising administering to a subject in need thereof a therapeutically effective amount of tenofovir alafenamide hemifumarate. In another embodiment is provided a method for treating an HBV infection comprising administering to a subject in need thereof a therapeutically effective amount of the pharmaceutical composition comprising tenofovir alafenamide hemifumarate.

In one embodiment is provided a method for preparing a pharmaceutical composition comprising combining tenofovir alafenamide hemifumarate and a pharmaceutically acceptable excipient to provide the pharmaceutical composition.

In one embodiment is provided tenofovir alafenamide hemifumarate for use in medical therapy.

In one embodiment is provided the use of tenofovir alafenamide hemifumarate for the prophylactic or therapeutic treatment of an HIV infection. In another embodiment is provided the use of tenofovir alafenamide hemifumarate to treat an HIV infection. In a further embodiment is provided the use of tenofovir alafenamide hemifumarate for the preparation or manufacture of a medicament for the treatment of an HIV infection. In another further embodiment is provided tenofovir alafenamide hemifumarate for use in treating an HIV infection.

In one embodiment is provided the use of tenofovir alafenamide hemifumarate for the prophylactic or therapeutic treatment of an HBV infection. In another embodiment is provided the use of tenofovir alafenamide hemifumarate to treat an HBV infection. In a further embodiment is provided the use of tenofovir alafenamide hemifumarate for the preparation or manufacture of a medicament for the treatment of an HBV infection. In another further embodiment is provided tenofovir alafenamide hemifumarate for use in treating an HBV infection.

In some embodiments of the invention, the methods of treating and the like comprise administration of multiple daily doses. In other embodiments, the methods of treating and the like comprise administration of a single daily dose.

In one embodiment, the invention provides for the use of tenofovir alafenamide hemifumarate and cobicistat, or a pharmaceutically acceptable salt thereof, for the prophylactic or therapeutic treatment of a viral infection in a human. The cobicistat may be coadministered with tenofovir alafenamide hemifumarate. Tenofovir alafenamide hemifumarate may be used in amounts of 3 mg, 8±3 mg, 10±5 mg, 25±5 mg, or 40±10 mg or other ranges as set forth below. Cobicistat or a pharmaceutically acceptable salt thereof may be used in an amount of 50-500 mg, 100-400 mg, 100-300 mg or 150 mg. Tenofovir alafenamide hemifumarate and cobicistat or pharmaceutically acceptable salt thereof may be coadministered. A unit dosage form comprising a daily amount of tenofovir alafenamide hemifumarate, and a daily amount of cobicistat or pharmaceutically acceptable salt thereof may be used. The virus may be human immunodeficiency virus (HIV) or Hepatitis B virus (HBV).

In one embodiment, the invention provides for the use tenofovir alafenamide hemifumarate and cobicistat, or a pharmaceutically acceptable salt thereof, for improving the pharmacokinetics of tenofovir alafenamide hemifumarate. Cobicistat may be coadministered with tenofovir alafenamide hemifumarate. Tenofovir alafenamide hemifumarate may be used in amounts of 3 mg, 8±3 mg, 10±5 mg, 25±5 mg, or 40±10 mg or other ranges as set forth below. Cobicistat or a pharmaceutically acceptable salt thereof may be used in an amount of 50-500 mg, 100-400 mg, 100-300 mg or 150 mg. Tenofovir alafenamide hemifumarate and cobicistat, or pharmaceutically acceptable salt thereof, may be coadministered. A unit dosage form comprising a daily amount of tenofovir alafenamide hemifumarate, and a daily amount of cobicistat or pharmaceutically acceptable salt thereof may be used. The use may be for the prophylactic or therapeutic treatment of a viral infection in a human. The virus may be human immunodeficiency virus (HIV) or Hepatitis B virus (HBV).

In one embodiment, the invention provides for the use of tenofovir alafenamide hemifumarate and cobicistat, or a pharmaceutically acceptable salt thereof, for improving the Cₘₐₓ of tenofovir alafenamide hemifumarate. The cobicistat may be coadministered with tenofovir alafenamide hemifumarate. Tenofovir alafenamide hemifumarate may be used in amounts of 3 mg, 8±3 mg, 10±5 mg, 25±5 mg, or 40±10 mg or other ranges as set forth below. Cobicistat or a pharmaceutically acceptable salt thereof may be used in an amount of 50-500 mg, 100-400 mg, 100-300 mg or 150 mg. Tenofovir alafenamide hemifumarate and cobicistat, or pharmaceutically acceptable salt thereof, may be coadministered. A unit dosage form comprising a daily amount of tenofovir alafenamide hemifumarate, and a daily amount of cobicistat, or a pharmaceutically acceptable salt thereof, may be used. The use may be for the prophylactic or therapeutic treatment of a viral infection in a human. The virus may be human immunodeficiency virus (HIV) or Hepatitis B virus (HBV).

In one embodiment, the invention provides for the use of tenofovir alafenamide hemifumarate and cobicistat, or a pharmaceutically acceptable salt thereof, for improving blood levels of tenofovir alafenamide hemifumarate. The cobicistat may be coadministered with tenofovir alafenamide hemifumarate. Tenofovir alafenamide hemifumarate may be used in amounts of 3 mg, 8±3 mg, 10±5 mg, 25±5 mg, or 40±10 mg or other ranges as set forth below. Cobicistat, or a pharmaceutically acceptable salt thereof, may be used in an amount of 50-500 mg, 100-400 mg, 100-300 mg or 150 mg. Tenofovir alafenamide hemifumarate and cobicistat, or a pharmaceutically acceptable salt thereof, may be coadministered. A unit dosage form comprising a daily amount tenofovir alafenamide hemifumarate, and a daily amount cobicistat, or a pharmaceutically acceptable salt thereof, may be used. The use may be for the prophylactic or therapeutic treatment of a viral infection in a human. The virus may be human immunodeficiency virus (HIV) or Hepatitis B virus (HBV).

In one embodiment, the invention provides for a composition comprising a unit-dosage form of tenofovir alafenamide hemifumarate; a unit-dosage form of cobicistat, or a pharmaceutically acceptable salt thereof; and a pharmaceutically acceptable carrier or diluent. The composition may include tenofovir alafenamide hemifumarate in amounts of 3 mg, 8±3 mg, 10±5 mg, 25±5 mg, or 40±10 mg or other ranges as set forth below. The composition may include cobicistat in amounts of 50-500 mg, 100-400 mg, 100-300 mg or 150 mg. The unit-dosage form may be a single daily dosage.

In one embodiment, the invention provides for a kit comprising: (1) tenofovir alafenamide hemifumarate; (2) cobicistat, or a pharmaceutically acceptable salt thereof; (3) one or more containers; and (4) prescribing information regarding administering the tenofovir alafenamide hemifumarate with the cobicistat, or the pharmaceutically acceptable salt thereof. The kit may include tenofovir alafenamide hemifumarate in amounts of 3 mg, 8±3 mg, 10±5 mg, 25±5 mg, or 40±10 mg or other ranges as set forth below. The kit may include cobicistat in amounts of 50-500 mg, 100-400 mg, 100-300 mg or 150 mg.

In one embodiment, the invention provides for a method of treating a viral infection in a human comprising coadministering tenofovir alafenamide hemifumarate with cobicistat, or a pharmaceutically acceptable salt thereof, wherein the dose of cobicistat coadministered with the tenofovir alafenamide hemifumarate provides a systemic exposure of tenofovir alafenamide hemifumarate comparable to the systemic exposure obtainable by administration of a greater dose of tenofovir alafenamide hemifumarate in the absence of cobicistat. Tenofovir alafenamide hemifumarate in amounts of 3 mg, 8±3 mg, 10±5 mg, 25±5 mg, or 40±10 mg or other ranges as set forth below may be coadministered with cobicistat. Cobicistat in amounts of 50-500 mg, 100-400 mg, 100-300 mg or 150 mg may be coadministered with tenofovir alafenamide hemifumarate. The virus may be human immunodeficiency virus (HIV) or Hepatitis B virus (HBV).

In one embodiment, the invention provides for a method for inhibiting activity of a retroviral reverse transcriptase in a human comprising coadministering tenofovir alafenamide hemifumarate with cobicistat, or a pharmaceutically acceptable salt thereof, wherein the dose of cobicistat coadministered with the tenofovir alafenamide hemifumarate provides a systemic exposure of tenofovir alafenamide hemifumarate comparable to the systemic exposure obtainable by administration of a greater dose of tenofovir alafenamide hemifumarate in the absence of cobicistat. Tenofovir alafenamide hemifumarate or a pharmaceutically acceptable salt thereof in amounts of 3 mg, 8±3 mg, 10±5 mg, 25±5 mg, or 40±10 mg or other ranges as set forth below may be coadministered with cobicistat. Cobicistat in amounts of 50-500 mg, 100-400 mg, 100-300 mg or 150 mg may be coadministered with tenofovir alafenamide hemifumarate. The virus may be human immunodeficiency virus (HIV).

In one embodiment, the invention provides for the use of tenofovir alafenamide hemifumarate coadministered with cobicistat, or a pharmaceutically acceptable salt thereof for the manufacture of a medicament for treating a viral infection. The invention further provides for the use of tenofovir alafenamide hemifumarate coadministered with cobicistat, or a pharmaceutically acceptable salt thereof for the manufacture of a medicament for treating a viral infection in a human. Tenofovir alafenamide hemifumarate may be used in a subtherapeutic amount (or, in some embodiments throughout, in a therapeutic amount). Tenofovir alafenamide hemifumarate may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. Cobicistat may be used in an amount that provides a systemic exposure of tenofovir alafenamide hemifumarate comparable to the systemic exposure obtainable by administration of a greater dose of tenofovir alafenamide hemifumarate in the absence of cobicistat in the manufacture of the medicament. The virus may be human immunodeficiency virus (HIV) or Hepatitis B virus (HBV).

In one embodiment, the invention provides for the use of tenofovir alafenamide hemifumarate coadministered with cobicistat, or a pharmaceutically acceptable salt thereof for the manufacture of a medicament for inhibiting activity of a retroviral reverse transcriptase. The invention further provides for the use of tenofovir alafenamide hemifumarate coadministered with cobicistat, or a pharmaceutically acceptable salt thereof, for the manufacture of a medicament for inhibiting activity of a retroviral reverse transcriptase in a human. Tenofovir alafenamide hemifumarate may be used in a subtherapeutic amount. Tenofovir alafenamide hemifumarate may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. Cobicistat may be used in an amount that provides a systemic exposure of tenofovir alafenamide hemifumarate comparable to the systemic exposure obtainable by administration of a greater dose of tenofovir alafenamide hemifumarate in the absence of cobicistat in the manufacture of the medicament. The virus may be human immunodeficiency virus (HIV).

In one embodiment, the invention provides for the use of cobicistat, or a pharmaceutically acceptable salt thereof, to prepare a medicament useful for improving the pharmacokinetics of tenofovir alafenamide hemifumarate following administration to a human. Tenofovir alafenamide hemifumarate may be used in a subtherapeutic amount. Tenofovir alafenamide hemifumarate may be used in amounts of 3 mg, 8±3 mg, 10±5 mg, 25±5 mg, or 40±10 mg or other ranges as set forth below. Cobicistat may be used in amounts of 50-500 mg, 100-400 mg, 100-300 mg or 150 mg. Cobicistat may be used in an amount that provides a systemic exposure of tenofovir alafenamide hemifumarate comparable to the systemic exposure obtainable by administration of a greater dose of tenofovir alafenamide hemifumarate in the absence of cobicistat in the manufacture of the medicament. The medicament may be used for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of tenofovir alafenamide hemifumarate by about 30-70% upon administration of the cobicistat. The use may be for the prophylactic or therapeutic treatment of a viral infection in a human. The virus may be human immunodeficiency virus (HIV) or Hepatitis B virus (HBV).

In one embodiment, the invention provides for the use of cobicistat, or a pharmaceutically acceptable salt thereof; to prepare a medicament for a human useful for reducing a dose of tenofovir alafenamide hemifumarate by about 2-4 fold upon administration of the cobicistat. In one embodiment, the invention provides for the use of cobicistat, or a pharmaceutically acceptable salt thereof; to prepare a medicament for a human useful for reducing a dose of tenofovir alafenamide hemifumarate by about 3 fold upon administration of the cobicistat. The use may be for the prophylactic or therapeutic treatment of a viral infection in a human. The virus may be human immunodeficiency virus (HIV) or Hepatitis B virus (HBV).

In one embodiment, the invention provides for a method of treating a viral infection in a human comprising coadministering 1) tenofovir alafenamide hemifumarate; and 2) cobicistat, or a pharmaceutically acceptable salt thereof, to the human. Tenofovir alafenamide hemifumarate is administered in a subtherapeutic amount. The virus may be human immunodeficiency virus (HIV) or Hepatitis B virus (HBV).

In one embodiment, the invention provides for a use of a subtherapeutic dose of tenofovir alafenamide hemifumarate coadministered with cobicistat for treating a viral infection. The virus may be human immunodeficiency virus (HIV) or Hepatitis B virus (HBV).

In one embodiment, the invention provides for the use of a subtherapeutic dose of tenofovir alafenamide hemifumarate coadministered with cobicistat for inhibiting retroviral reverse transcriptase. The virus may be human immunodeficiency virus (HIV)

In one embodiment, the invention provides for an anti-virus agent(s) comprising (a) tenofovir alafenamide hemifumarate and (b) cobicistat, or a pharmaceutically acceptable salt thereof. The anti-virus agent(s) may include tenofovir alafenamide hemifumarate in amounts of 3 mg, 8±3 mg, 10±5 mg, 25±5 mg, or 40±10 mg or other ranges as set forth below. The anti-virus agent(s) may include cobicistat in amounts of 50-500 mg, 100-400 mg, 100-300 mg or 150 mg. The cobicistat may be used in an amount that provides a systemic exposure of tenofovir alafenamide hemifumarate comparable to the systemic exposure obtainable by administration of a greater dose of tenofovir alafenamide hemifumarate in the absence of cobicistat in the manufacture of the medicament. The anti-virus agent may further include 200 mg of emtricitabine and 150 mg of elvitegravir. The anti-virus agent may further include 150 mg cobicistat, 8 or less mg tenofovir alafenamide hemifumarate, 150 mg elvitegravir, and 200 mg emtricitabine. The anti-virus agent may further include 150 mg cobicistat, 25 or less mg tenofovir alafenamide hemifumarate, 150 mg elvitegravir, and 200 mg emtricitabine. The anti-virus agent may further include 150 mg cobicistat, 10 or less mg tenofovir alafenamide hemifumarate, 150 mg elvitegravir, and 200 mg emtricitabine. The anti-virus agent may include 150 mg cobicistat, 8 mg tenofovir alafenamide hemifumarate, 150 mg elvitegravir, and 200 mg emtricitabine. The anti-virus agent may include 150 mg cobicistat, 10 mg tenofovir alafenamide hemifumarate, 150 mg elvitegravir, and 200 mg emtricitabine.

In one embodiment, the invention provides for a unit-dosage of tenofovir alafenamide hemifumarate and cobicistat, or a pharmaceutically acceptable salt thereof, wherein the unit-dosage is a daily dose. Tenofovir alafenamide hemifumarate may be present in a subtherapeutic amount. The unit-dosage may further include 150 mg cobicistat, 8 or less mg tenofovir alafenamide hemifumarate, 150 mg elvitegravir, and 200 mg emtricitabine. The unit-dosage may further include 150 mg cobicistat, 25 or less mg tenofovir alafenamide hemifumarate, 150 mg elvitegravir, and 200 mg emtricitabine. The unit-dosage may further include 150 mg cobicistat, 10 or less mg tenofovir alafenamide hemifumarate, 150 mg elvitegravir, and 200 mg emtricitabine. The unit-dosage may include 150 mg cobicistat, 10 mg tenofovir alafenamide hemifumarate, 150 mg elvitegravir, and 200 mg emtricitabine.

In one embodiment, the invention provides the use of cobicistat, or a pharmaceutically acceptable salt thereof; to prepare a medicament useful for improving the pharmacokinetics of tenofovir alafenamide hemifumarate following administration to a human. The medicament may be used for the prophylactic or therapeutic treatment of a viral infection in a human. The virus may be human immunodeficiency virus (HIV) or Hepatitis B virus (HBV).

In one embodiment, the invention provides cobicistat for use in improving the pharmacokinetics of tenofovir alafenamide hemifumarate following administration to a human. The use may be for the prophylactic or therapeutic treatment of a viral infection in a human. The virus may be human immunodeficiency virus (HIV) or Hepatitis B virus (HBV).

In one embodiment, the invention provides a kit comprising: (1) tenofovir alafenamide hemifumarate; (2) cobicistat, or a pharmaceutically acceptable salt thereof; (3) one or more containers; and (4) prescribing information regarding administering the tenofovir alafenamide hemifumarate with the cobicistat or a pharmaceutically acceptable salt thereof.

In one embodiment, the invention provides a kit comprising: (1) a unit dosage form comprising 5-100 mg of tenofovir alafenamide hemifumarate; (2) a unit dosage form comprising 150 mg cobicistat, or a pharmaceutically acceptable salt thereof; (3) one or more containers; and (4) prescribing information regarding administering the tenofovir alafenamide hemifumarate with cobicistat or a pharmaceutically acceptable salt thereof.

In one embodiment, the invention provides a use of tenofovir alafenamide hemifumarate for the manufacture of a medicament for inhibiting activity of a retroviral reverse transcriptase in a human, comprising administering tenofovir alafenamide hemifumarate and cobicistat, or a pharmaceutically acceptable salt thereof, to the human. The virus may be human immunodeficiency virus (HIV).

In one embodiment, the invention provides tenofovir alafenamide hemifumarate and cobicistat, or a pharmaceutically acceptable salt thereof, for use in inhibiting activity of a retroviral reverse transcriptase in a human.

In one embodiment, the invention provides a use of cobicistat, or a pharmaceutically acceptable salt thereof, to prepare a medicament for a human useful for reducing a dose between about 30-70% of tenofovir alafenamide hemifumarate upon administration of the cobicistat. The medicament may be used for the prophylactic or therapeutic treatment of a viral infection in a human. The virus may be human immunodeficiency virus (HIV) or Hepatitis B virus (HBV).

In one embodiment, the invention provides the use of tenofovir alafenamide hemifumarate and cobicistat, or a pharmaceutically acceptable salt thereof, for the prophylactic or therapeutic treatment of a viral infection in a human. The use may be for the prophylactic or therapeutic treatment of a viral infection in a human. The virus may be human immunodeficiency virus (HIV) or Hepatitis B virus (HBV).

In one embodiment, the invention provides an anti-viral agent(s) comprising (a) tenofovir alafenamide hemifumarate, which is used in combination with (b) cobicistat, or a pharmaceutically acceptable salt thereof, for use in the prophylactic or therapeutic treatment of a viral infection in a human.

In one embodiment, the invention provides for the use of ritonavir in the compositions, kits, unit-dosages and uses set forth above in place of cobicistat.

In one embodiment, the invention provides a method for inhibiting Pgp-mediated intestinal secretion of GS-7340, or a pharmaceutically acceptable salt thereof, in a human by coadministration of cobicistat, or a pharmaceutically acceptable salt thereof, with GS-7340, or a pharmaceutically acceptable salt thereof. In one embodiment, 150 mg of cobicistat, or a pharmaceutically acceptable salt thereof, is coadministered with 10 mg of GS-7340, or a pharmaceutically acceptable salt thereof.

In one embodiment, the invention provides a method for inhibiting Pgp-mediated intestinal secretion of tenofovir alafenamide hemifumarate in a human by coadministration of cobicistat, or a pharmaceutically acceptable salt thereof, with tenofovir alafenamide hemifumarate. In one embodiment, 150 mg of cobicistat, or a pharmaceutically acceptable salt thereof, is coadministered with 10 mg of tenofovir alafenamide hemifumarate.

In one embodiment, the invention provides the use of an anti-virus agent for the prophylactic or therapeutic treatment of a viral infection in a human, wherein the anti-virus agent comprises 150 mg cobicistat, 10 or less mg GS-7340, 150 mg elvitegravir, and 200 mg emtricitabine.

In one embodiment, the invention provides a method of treating a viral infection in a human comprising coadministering 150 mg cobicistat, 10 or less mg GS-7340, 150 mg elvitegravir, and 200 mg emtricitabine to the human.

In one embodiment, the invention provides the use of 150 mg cobicistat, 10 or less mg GS-7340, 150 mg elvitegravir, and 200 mg emtricitabine for the manufacture of a medicament for treating a viral infection in a human.

In one embodiment, the invention provides the use of an anti-virus agent for the prophylactic or therapeutic treatment of a viral infection in a human, wherein the anti-virus agent comprises 150 mg cobicistat, 10 or less mg tenofovir alafenamide hemifumarate, 150 mg elvitegravir, and 200 mg emtricitabine.

In one embodiment, the invention provides a method of treating a viral infection in a human comprising coadministering 150 mg cobicistat, 10 or less mg tenofovir alafenamide hemifumarate, 150 mg elvitegravir, and 200 mg emtricitabine to the human.

In one embodiment, the invention provides the use of 150 mg cobicistat, 10 or less mg tenofovir alafenamide hemifumarate, 150 mg elvitegravir, and 200 mg emtricitabine for the manufacture of a medicament for treating a viral infection in a human.

In one embodiment, the invention provides an anti-virus agent(s) comprising (a) tenofovir alafenamide hemifumarate, (b) cobicistat, or a pharmaceutically acceptable salt thereof, (c) emtricitabine, and (d) darunavir.

In one embodiment, the invention provides an anti-virus agent(s) comprising (a) 8 or less mg of tenofovir alafenamide hemifumarate, (b) 150 mg of cobicistat, or a pharmaceutically acceptable salt thereof, (c) 200 mg of emtricitabine, and (d) 800 mg of darunavir.

In one embodiment, the invention provides an anti-virus agent(s) comprising (a) 25 or less mg of tenofovir alafenamide hemifumarate, (b) 150 mg of cobicistat, or a pharmaceutically acceptable salt thereof, (c) 200 mg of emtricitabine, and (d) 800 mg of darunavir.

In one embodiment, the invention provides an anti-virus agent(s) comprising (a) 10 mg of tenofovir alafenamide hemifumarate, (b) 150 mg of cobicistat, or a pharmaceutically acceptable salt thereof, (c) 200 mg of emtricitabine, and (d) 800 mg of darunavir.

In one embodiment, the invention provides an anti-virus agent(s) comprising (a) GS-7340, or a pharmaceutically acceptable salt thereof, (b) cobicistat, or a pharmaceutically acceptable salt thereof, (c) emtricitabine, and (d) darunavir.

In one embodiment, the invention provides an anti-virus agent(s) comprising (a) 8 or less mg of GS-7340, or a pharmaceutically acceptable salt thereof, (b) 150 mg of cobicistat, or a pharmaceutically acceptable salt thereof, (c) 200 mg of emtricitabine, and (d) 800 mg of darunavir.

In one embodiment, the invention provides an anti-virus agent(s) comprising (a) 25 or less mg of GS-7340, or a pharmaceutically acceptable salt thereof, (b) 150 mg of cobicistat, or a pharmaceutically acceptable salt thereof, (c) 200 mg of emtricitabine, and (d) 800 mg of darunavir.

In one embodiment, the invention provides an anti-virus agent(s) comprising (a) 10 mg of GS-7340, or a pharmaceutically acceptable salt thereof, (b) 150 mg of cobicistat, or a pharmaceutically acceptable salt thereof, (c) 200 mg of emtricitabine, and (d) 800 mg of darunavir.

In one embodiment, the invention provides the use of an anti-virus agent for the prophylactic or therapeutic treatment of a viral infection in a human, wherein the anti-virus agent comprises 150 mg cobicistat, 10 or less mg GS-7340, 800 mg of darunavir, and 200 mg emtricitabine.

In one embodiment, the invention provides a method of treating a viral infection in a human comprising coadministering 150 mg cobicistat, 10 or less mg GS-7340, 800 mg of darunavir, and 200 mg emtricitabine to the human.

In one embodiment, the invention provides the use of 150 mg cobicistat, 10 or less mg GS-7340, 800 mg of darunavir, and 200 mg emtricitabine for the manufacture of a medicament for treating a viral infection in a human.

In one embodiment, the invention provides the use of an anti-virus agent for the prophylactic or therapeutic treatment of a viral infection in a human, wherein the anti-virus agent comprises 150 mg cobicistat, 10 or less mg tenofovir alafenamide hemifumarate, 800 mg of darunavir, and 200 mg emtricitabine.

In one embodiment, the invention provides a method of treating a viral infection in a human comprising coadministering 150 mg cobicistat, 10 or less mg tenofovir alafenamide hemifumarate, 800 mg of darunavir, and 200 mg emtricitabine to the human.

In one embodiment, the invention provides the use of 150 mg cobicistat, 10 or less mg tenofovir alafenamide hemifumarate, 800 mg of darunavir, and 200 mg emtricitabine for the manufacture of a medicament for treating a viral infection in a human.

In one embodiment, the invention provides the use of a dose of a cytochrome p450 inhibitor, or a pharmaceutically acceptable salt thereof, to boost a dose GS-7340, or a pharmaceutically acceptable salt thereof, for the prophylactic or therapeutic treatment of a viral infection in a human. In one embodiment, the cytochrome p450 inhibitor is cobicistat, or a pharmaceutically acceptable salt thereof. In one further embodiment, the dose of GS-7340 would be a subtherapeutic amount absent the dose of cobicistat.

In one embodiment, the invention provides a composition comprising: a unit-dosage form of GS-7340, or a pharmaceutically acceptable salt thereof; a unit-dosage form of cobicistat, or a pharmaceutically acceptable salt thereof; and a pharmaceutically acceptable carrier or diluent, wherein the amount of GS-7340 in the unit-dosage form is a subtherapeutic amount.

In one embodiment, the invention provides the use of a dose of a cytochrome p450 inhibitor, or a pharmaceutically acceptable salt thereof, to boost a dose tenofovir alafenamide hemifumarate for the prophylactic or therapeutic treatment of a viral infection in a human. In one embodiment, the cytochrome p450 inhibitor is cobicistat, or a pharmaceutically acceptable salt thereof. In one further embodiment, the dose of tenofovir alafenamide hemifumarate would be a subtherapeutic amount absent the dose of cobicistat.

In one embodiment, the invention provides a composition comprising: a unit-dosage form of tenofovir alafenamide hemifumarate; a unit-dosage form of cobicistat, or a pharmaceutically acceptable salt thereof; and a pharmaceutically acceptable carrier or diluent, wherein the amount of tenofovir alafenamide hemifumarate in the unit-dosage form is a subtherapeutic amount.

In one embodiment, the invention provides the uses and methods related to treating a viral infection, as noted herein, wherein the viral infection is human immunodeficiency virus (HIV).

In one embodiment, the invention provides the uses and methods related to treating a viral infection, as noted herein, wherein the viral infection is Hepatitis B virus (HBV).

In one embodiment, the invention provides a method of treating a viral infection in a human, comprising administering to the human a composition comprising cobicistat, or a pharmaceutically acceptable salt thereof, and tenofovir alafenamide hemifumarate, wherein the composition contains an amount of cobicistat, or a pharmaceutically acceptable salt thereof, sufficient for an amount of tenofovir alafenamide hemifumarate in the composition to provide an effect on the viral infection that is greater than the effect of the amount of tenofovir alafenamide hemifumarate in the absence of cobicistat, or a pharmaceutically acceptable salt thereof, and wherein the viral infection is human immunodeficiency virus (HIV) or Hepatitis B virus (HBV).

In one embodiment, the invention provides a method of treating a viral infection in a human, comprising administering to the human a composition comprising cobicistat, or a pharmaceutically acceptable salt thereof, and tenofovir alafenamide hemifumarate, wherein an effect on the viral infection of an amount of tenofovir alafenamide hemifumarate in the composition is greater than the effect of the same amount of tenofovir alafenamide hemifumarate in the absence of cobicistat, or a pharmaceutically acceptable salt thereof, and wherein the viral infection is human immunodeficiency virus (HIV) or Hepatitis B virus (HBV).

In one embodiment, the invention provides an anti-viral treatment method on a viral infection in a human, comprising administering to the human a composition comprising cobicistat, or a pharmaceutically acceptable salt thereof, and tenofovir alafenamide hemifumarate, wherein the composition contains an amount of cobicistat, or a pharmaceutically acceptable salt thereof, sufficient for an amount of tenofovir alafenamide hemifumarate in the composition to provide an anti-viral effect that is greater than the anti-viral effect of the amount of tenofovir alafenamide hemifumarate in the absence of cobicistat, or a pharmaceutically acceptable salt thereof, and wherein the viral infection is human immunodeficiency virus (HIV) or Hepatitis B virus (HBV).

In one embodiment, the invention provides an anti-viral treatment method on a viral infection in a human, comprising administering to the human a composition comprising cobicistat, or a pharmaceutically acceptable salt thereof, and tenofovir alafenamide hemifumarate, wherein an anti-viral effect of an amount of tenofovir alafenamide hemifumarate in the composition is greater than the anti-viral effect of the same amount of tenofovir alafenamide hemifumarate in the absence of cobicistat, or a pharmaceutically acceptable salt thereof, and wherein the viral infection is human immunodeficiency virus (HIV) or Hepatitis B virus (HBV).

In one embodiment, the invention provides a composition comprising: cobicistat, or a pharmaceutically acceptable salt thereof; and tenofovir alafenamide hemifumarate. In a further embodiment, the composition comprises: 50-500 mg of cobicistat, or a pharmaceutically acceptable salt thereof; and 3-40 mg of tenofovir alafenamide hemifumarate. In another embodiment, the composition further comprises a pharmaceutically acceptable carrier or diluent.

In one embodiment, the invention provides a method of treating a viral infection in a human comprising administering a composition comprising: cobicistat, or a pharmaceutically acceptable salt thereof; and tenofovir alafenamide hemifumarate, to the human.

In one embodiment, the invention provides a method of treating a viral infection in a human comprising coadministering cobicistat, or a pharmaceutically acceptable salt thereof, and tenofovir alafenamide hemifumarate, to the human.

In one embodiment, the invention provides a method of inhibiting activity of a retroviral reverse transcriptase comprising coadministering cobicistat, or a pharmaceutically acceptable salt thereof, and tenofovir alafenamide hemifumarate. In a further embodiment, the coadministering of cobicistat, or a pharmaceutically acceptable salt thereof, and tenofovir alafenamide hemifumarate, is in a human.

In one embodiment, the invention provides use of cobicistat, or a pharmaceutically acceptable salt thereof, and tenofovir alafenamide hemifumarate, for the prophylactic or therapeutic treatment of a viral infection in a human.

In one embodiment, the invention provides use of cobicistat, or a pharmaceutically acceptable salt thereof, and tenofovir alafenamide hemifumarate, for the manufacture of a medicament for treating a viral infection in a human.

In one embodiment, the invention provides use of cobicistat, or a pharmaceutically acceptable salt thereof, and tenofovir alafenamide hemifumarate, for the manufacture of a medicament for inhibiting activity of a retroviral reverse transcriptase. In a further embodiment, the medicament is for inhibiting activity of a retroviral reverse transcriptase in a human.

In one embodiment, the invention provides a method of boosting an anti-viral effect of tenofovir alafenamide hemifumarate in a human comprising administering a composition comprising: cobicistat, or a pharmaceutically acceptable salt thereof; and tenofovir alafenamide hemifumarate, to the human.

In one embodiment, the invention provides a method of boosting an anti-viral effect of tenofovir alafenamide hemifumarate in a human comprising coadministering cobicistat, or a pharmaceutically acceptable salt thereof, and tenofovir alafenamide hemifumarate to the human. In a further embodiment, 50-500 mg of cobicistat, or a pharmaceutically acceptable salt thereof, is coadministered with 3-40 mg of tenofovir alafenamide hemifumarate.

In one embodiment, the invention provides a method of inhibiting Pgp-mediated intestinal secretion of tenofovir alafenamide hemifumarate in a human comprising administering a composition comprising: cobicistat, or a pharmaceutically acceptable salt thereof; and tenofovir alafenamide hemifumarate, to the human.

In one embodiment, the invention provides a method of inhibiting Pgp-mediated intestinal secretion of tenofovir alafenamide hemifumarate in a human by coadministration of cobicistat, or a pharmaceutically acceptable salt thereof, and tenofovir alafenamide hemifumarate. In a further embodiment, 50-500 mg of cobicistat, or a pharmaceutically acceptable salt thereof, is coadministered with 3-40 mg of tenofovir alafenamide hemifumarate.

In additional embodiments, the invention provides the methods and uses disclosed wherein the viral infection is human immunodeficiency virus (HIV) or Hepatitis B virus (HBV).

In one embodiment, the invention provides a composition comprising: (a) tenofovir alafenamide hemifumarate; (b) cobicistat, or a pharmaceutically acceptable salt thereof; (c) emtricitabine; and (d) elvitegravir. In a further embodiment, the composition comprises: (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir. In a further embodiment, the invention provides a method of treating a viral infection in a human comprising administering such a composition to the human.

In one embodiment, the invention provides a method of treating a viral infection in a human comprising coadministering (a) tenofovir alafenamide hemifumarate; (b) cobicistat, or a pharmaceutically acceptable salt thereof; (c) emtricitabine; and (d) elvitegravir to the human. In a further embodiment, the method comprises coadministering (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir to the human.

In one embodiment, the invention provides use of a composition comprising: (a) tenofovir alafenamide hemifumarate; (b) cobicistat, or a pharmaceutically acceptable salt thereof; (c) emtricitabine; and (d) elvitegravir, for the prophylactic or therapeutic treatment of a viral infection in a human.

In one embodiment, the invention provides use of (a) tenofovir alafenamide hemifumarate; (b) cobicistat, or a pharmaceutically acceptable salt thereof; (c) emtricitabine; and (d) elvitegravir for the manufacture of a medicament for treating a viral infection in a human. In a further embodiment, the invention provides use of (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir for the manufacture of a medicament for treating a viral infection in a human.

In one embodiment, the invention provides a composition comprising: (a) tenofovir alafenamide hemifumarate; (b) cobicistat, or a pharmaceutically acceptable salt thereof; (c) emtricitabine; and (d) elvitegravir for the treatment of a viral infection, wherein the viral infection is human immunodeficiency virus (HIV) or Hepatitis B virus (HBV).

In one embodiment, the invention provides a composition comprising: (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir for the treatment of a viral infection, wherein the viral infection is human immunodeficiency virus (HIV) or Hepatitis B virus (HBV).

In additional embodiments, the invention provides the methods and uses disclosed wherein the viral infection is human immunodeficiency virus (HIV) or Hepatitis B virus (HBV).

In one embodiment, the invention provides a composition comprising: (a) tenofovir alafenamide hemifumarate; (b) cobicistat, or a pharmaceutically acceptable salt thereof; (c) emtricitabine; and (d) darunavir. In a further embodiment, the composition comprises: (a) 3-40 mg tenofovir alafenamide hemifumarate; (b) 50-500 mg cobicistat, or a pharmaceutically acceptable salt thereof; (c) 50-500 mg emtricitabine; and (d) 400-1600 mg darunavir. In a further embodiment, the invention provides a method of treating a viral infection in a human comprising administering such a composition to the human.

In one embodiment, the invention provides a method of treating a viral infection in a human comprising coadministering (a) tenofovir alafenamide hemifumarate; (b) cobicistat, or a pharmaceutically acceptable salt thereof; (c) emtricitabine; and (d) darunavir to the human. In a further embodiment, the method comprises coadministering (a) 3-40 mg tenofovir alafenamide hemifumarate; (b) 50-500 mg cobicistat, or a pharmaceutically acceptable salt thereof; (c) 50-500 mg emtricitabine; and (d) 400-1600 mg darunavir to the human.

In one embodiment, the invention provides use of a composition comprising: (a) tenofovir alafenamide hemifumarate; (b) cobicistat, or a pharmaceutically acceptable salt thereof; (c) emtricitabine; and (d) darunavir, for the prophylactic or therapeutic treatment of a viral infection in a human.

In one embodiment, the invention provides use of (a) tenofovir alafenamide hemifumarate; (b) cobicistat, or a pharmaceutically acceptable salt thereof; (c) emtricitabine; and (d) darunavir for the manufacture of a medicament for treating a viral infection in a human. In a further embodiment, the invention provides use of (a) 3-40 mg tenofovir alafenamide hemifumarate; (b) 50-500 mg cobicistat, or a pharmaceutically acceptable salt thereof; (c) 50-500 mg emtricitabine; and (d) 400-1600 mg darunavir for the manufacture of a medicament for treating a viral infection in a human.

In one embodiment, the invention provides a composition comprising: (a) tenofovir alafenamide hemifumarate; (b) cobicistat, or a pharmaceutically acceptable salt thereof; (c) emtricitabine; and (d) darunavir for the treatment of a viral infection, wherein the viral infection is human immunodeficiency virus (HIV) or Hepatitis B virus (HBV).

In one embodiment, the invention provides a composition comprising: (a) 3-40 mg tenofovir alafenamide hemifumarate; (b) 50-500 mg cobicistat, or a pharmaceutically acceptable salt thereof; (c) 50-500 mg emtricitabine; and (d) 400-1600 mg darunavir for the treatment of a viral infection, wherein the viral infection is human immunodeficiency virus (HIV) or Hepatitis B virus (HBV).

In additional embodiments, the invention provides the methods and uses disclosed wherein the viral infection is human immunodeficiency virus (HIV) or Hepatitis B virus (HBV).

In one embodiment, the invention provides a composition comprising: tenofovir alafenamide hemifumarate and emtricitabine. In a further embodiment, the composition comprises: 3-40 mg tenofovir alafenamide hemifumarate and 50-500 mg emtricitabine. In a further embodiment, the invention provides a method of treating a viral infection in a human comprising administering such a composition to the human.

In one embodiment, the invention provides a method of treating a viral infection in a human comprising coadministering tenofovir alafenamide hemifumarate and emtricitabine to the human. In a further embodiment, the method comprises coadministering 3-40 mg tenofovir alafenamide hemifumarate and 50-500 mg emtricitabine to the human.

In one embodiment, the invention provides use of a composition comprising: tenofovir alafenamide hemifumarate and emtricitabine for the prophylactic or therapeutic treatment of a viral infection in a human.

In one embodiment, the invention provides use of tenofovir alafenamide hemifumarate and emtricitabine for the manufacture of a medicament for treating a viral infection in a human. In a further embodiment, the invention provides use of 3-40 mg tenofovir alafenamide hemifumarate and 50-500 mg emtricitabine for the manufacture of a medicament for treating a viral infection in a human.

In one embodiment, the invention provides a composition comprising: tenofovir alafenamide hemifumarate and emtricitabine for the treatment of a viral infection, wherein the viral infection is human immunodeficiency virus (HIV) or Hepatitis B virus (HBV).

In one embodiment, the invention provides a composition comprising: 3-40 mg tenofovir alafenamide hemifumarate and 50-500 mg emtricitabine for the treatment of a viral infection, wherein the viral infection is human immunodeficiency virus (HIV) or Hepatitis B virus (HBV).

In additional embodiments, the invention provides the methods and uses disclosed wherein the viral infection is human immunodeficiency virus (HIV) or Hepatitis B virus (HBV).

In one embodiment, the invention provides a composition comprising: (a) tenofovir alafenamide hemifumarate; (b) rilpivirine; and (c) emtricitabine. In a further embodiment, the composition comprises: (a) 3-40 mg tenofovir alafenamide hemifumarate; (b) 10-80 mg rilpivirine; and (c) 50-500 mg emtricitabine. In a further embodiment, the invention provides a method of treating a viral infection in a human comprising administering such a composition to the human.

In one embodiment, the invention provides a method of treating a viral infection in a human comprising coadministering (a) tenofovir alafenamide hemifumarate; (b) rilpivirine; and (c) emtricitabine to the human. In a further embodiment, the method comprises coadministering (a) 3-40 mg tenofovir alafenamide hemifumarate; (b) 10-80 mg rilpivirine; and (c) 50-500 mg emtricitabine to the human.

In one embodiment, the invention provides use of a composition comprising: (a) tenofovir alafenamide hemifumarate; (b) rilpivirine; and (c) emtricitabine, for the prophylactic or therapeutic treatment of a viral infection in a human.

In one embodiment, the invention provides use of (a) tenofovir alafenamide hemifumarate; (b) rilpivirine; and (c) emtricitabine for the manufacture of a medicament for treating a viral infection in a human. In a further embodiment, the invention provides use of (a) 3-40 mg tenofovir alafenamide hemifumarate; (b) 10-80 mg rilpivirine; and (c) 50-500 mg emtricitabine for the manufacture of a medicament for treating a viral infection in a human.

In one embodiment, the invention provides a composition comprising: (a) tenofovir alafenamide hemifumarate; (b) rilpivirine; and (c) emtricitabine for the treatment of a viral infection, wherein the viral infection is human immunodeficiency virus (HIV) or Hepatitis B virus (HBV).

In one embodiment, the invention provides a composition comprising: (a) 3-40 mg tenofovir alafenamide hemifumarate; (b) 10-80 mg rilpivirine; and (c) 50-500 mg emtricitabine for the treatment of a viral infection, wherein the viral infection is human immunodeficiency virus (HIV) or Hepatitis B virus (HBV).

In additional embodiments, the invention provides the methods and uses disclosed wherein the viral infection is human immunodeficiency virus (HIV) or Hepatitis B virus (HBV).

In one embodiment, the invention provides a composition comprising: tenofovir alafenamide hemifumarate and GS-9441. In a further embodiment, the composition comprises: 3-40 mg tenofovir alafenamide hemifumarate and 5-1500 mg GS-9441. In a further embodiment, the invention provides a method of treating a viral infection in a human comprising administering such a composition to the human.

In one embodiment, the invention provides a method of treating a viral infection in a human comprising coadministering tenofovir alafenamide hemifumarate and GS-9441 to the human. In a further embodiment, the method comprises coadministering 3-40 mg tenofovir alafenamide hemifumarate and 5-1500 mg GS-9441 to the human.

In one embodiment, the invention provides use of a composition comprising: tenofovir alafenamide hemifumarate and GS-9441 for the prophylactic or therapeutic treatment of a viral infection in a human.

In one embodiment, the invention provides use of tenofovir alafenamide hemifumarate and GS-9441 for the manufacture of a medicament for treating a viral infection in a human. In a further embodiment, the invention provides use of 3-40 mg tenofovir alafenamide hemifumarate and 5-1500 mg GS-9441 for the manufacture of a medicament for treating a viral infection in a human.

In one embodiment, the invention provides a composition comprising: tenofovir alafenamide hemifumarate and GS-9441 for the treatment of a viral infection, wherein the viral infection is human immunodeficiency virus (HIV) or Hepatitis B virus (HBV).

In one embodiment, the invention provides a composition comprising: 3-40 mg tenofovir alafenamide hemifumarate and 5-1500 mg GS-9441 for the treatment of a viral infection, wherein the viral infection is human immunodeficiency virus (HIV) or Hepatitis B virus (HBV).

In additional embodiments, the invention provides the methods and uses disclosed wherein the viral infection is human immunodeficiency virus (HIV) or Hepatitis B virus (HBV).

### BRIEF DESCRIPTION OF THE DRAWINGS

FIG. 1 shows pharmacokinetic data from patients dosed with various doses of GS-7340 and TDF.

FIG. 2 shows pharmacokinetic data from patients dosed with various doses of GS-7340 and TDF.

FIG. 3A-B shows pharmacokinetic data from patients dosed with various formulations of GS-7340.

FIG. 4A-B shows pharmacokinetic data from patients dosed with various formulations of GS-7340.

FIG. 5A-B shows pharmacokinetic data from patients dosed with various formulations of GS-7340.

FIG. 6 shows pharmacokinetic data from patients dosed with various formulations of GS-7340.

FIG. 7 shows pharmacokinetic data from patients dosed with various formulations of GS-7340.

FIG. 8 shows pharmacokinetic data from patients dosed with various formulations of GS-7340.

FIG. 9 shows pharmacokinetic data from patients dosed with various formulations of GS-7340.

FIG. 10A-B shows results of substrate assays in cells transfected with the genes for human P-glycoprotein (Pgp; MDR1) and breast cancer resistance protein (BCRP) genes.

FIG. 11A-B shows results of bidirectional permeability assays in cells transfected with the genes for human Pgp and BCRP.

FIG. 12A-F shows results of bidirectional permeability assays in cells transfected with the genes for human Pgp and BCRP.

FIG. 13 shows the X-ray powder diffraction (XRPD) pattern of tenofovir alafenamide hemifumarate.

FIG. 14 shows a graph of the DSC analysis of tenofovir alafenamide hemifumarate.

FIG. 15 shows a graph of the thermogravimetric analysis (TGA) data for tenofovir alafenamide hemifumarate.

FIG. 16 shows a graph of the dynamic vapor sorption (DVS) analysis of tenofovir alafenamide hemifumarate.

### DETAILED DESCRIPTION OF THE INVENTION

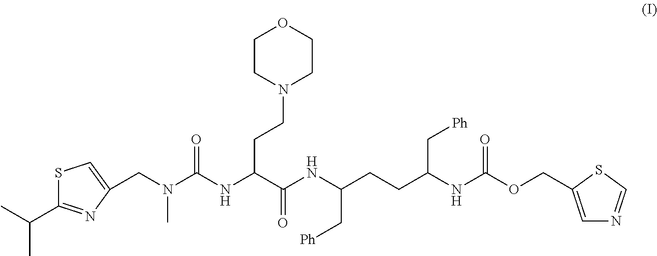 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

Cobicistat (chemical name 1,3-thiazol-5-ylmethyl (2R,5R)-(5-{[(2S)-2-[(methyl {[2-(propan-2-yl)-1,3-thiazol-4-yl]methyl}carbamoyl)amino]]-4-(morpholin-4-yl)butanamido}-1,6-diphenylhexan-2-yl)carbamate) is a chemical entity that has been shown to be a mechanism-based inhibitor that irreversibly inhibits CYP3A enzymes.

Detailed enzyme inactivation kinetic studies were performed comparing cobicistat with ritonavir. Cobicistat was found to be an efficient inactivator of human hepatic microsomal CYP3A activity with kinetic parameters similar to those of ritonavir. In addition, cobicistat is a moderate inhibitor of CYP2B6 (similar potency to ritonavir), a weak inhibitor of CYP2D6, and does not appreciably inhibit CYP1A2, CYP2C8, CYP2C9, CYP2C19, or uridine glucuronosyltransferase 1A1. In xenobiotic receptor transactivation and human hepatocyte studies, cobicistat displayed no/weak potential as an inducer of cytochrome P450, UGT1A1, or P-glycoprotein (at up to 30 μM). Permeability assays suggest that cobicistat is not a strong substrate or inhibitor of transporters including P-glycoprotein, MRP1, and MRP2. Inhibition of intestinal P-glycoprotein by cobicistat is only possible during absorption due to its high aqueous solubility, but it is not potent enough to inhibit transporters at systemic concentrations. These data indicate that, compared to ritonavir, cobicistat is a more selective inhibitor of CYP3A in vitro and a weaker inducer of CYP enzymes, which may potentially result in fewer clinically significant interactions with substrates of other CYP enzymes.

Cobicistat may also be present in compositions enriched with a stereoisomer of formula (Ia):

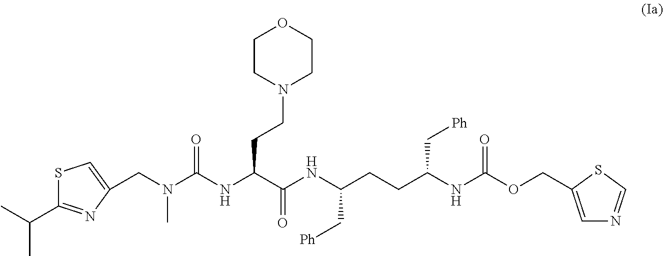 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

which is thiazol-5-ylmethyl (2R,5R)-5-((S)-2-(3-((2-isopropylthiazol-5-yl)methyl)-3-methylureido)-4-morpholinobutanamido)-1,6-diphenylhexan-2-ylcarbamate.

In one embodiment, the cobicistat has an enriched concentration of 85±5% of the stereoisomer of formula (Ia). In another embodiment, the cobicistat has an enriched concentration of 90±5% of the stereoisomer of formula (Ia). In another embodiment, the cobicistat has an enriched concentration of 95±2% of the stereoisomer of formula (Ia). In another embodiment, the cobicistat has an enriched concentration of 99±1% of the stereoisomer of formula (Ia). In another embodiment, the cobicistat is present as the pure stereoisomer of formula (Ia).

Coadministration of cobicistat with GS-7340 or tenofovir alafenamide hemifumarate boosts systemic exposure to GS-7340 or tenofovir alafenamide hemifumarate in humans, improves the pharmacokinetics of GS-7340 or tenofovir alafenamide hemifumarate (including, but not limited to, Cₘₐₓ increases), and increases blood levels of GS-7340/tenofovir alafenamide hemifumarate/tenofovir. Therefore, GS-7340 or tenofovir alafenamide hemifumarate coadministered with cobicistat may be administered in lower amounts than previously thought to achieve a therapeutic effect. Such lower amounts may be amounts that would be subtherapeutic in the absence of coadministration of cobicistat.

Without being bound by any theory of the invention, it is believed that cobicistat may be acting to inhibit intestinal Pgp-mediated intestinal secretion of GS-7340 or tenofovir alafenamide hemifumarate. In in vitro studies, cobicistat and ritonavir significantly increased the accumulation of probe substrates (such as calcein AM and Hoechst 33342) in cells transfected with P-glycoprotein (Pgp) and breast cancer resistance protein (BCRP), and cobicistat was found to be a substrate for these transporters. Cobicistat appears to be a substrate of Pgp and BCRP and likely has a competitive mode of inhibition with coadministered agents. Cobicistat appears to be a relatively weak inhibitor of Pgp and BCRP and may only have a transient effect on these transporters during intestinal absorption, facilitated by high solubility of, and resulting high concentrations of, cobicistat achievable in the gastrointestinal tract. Combined, these results suggest that cobicistat can effectively inhibit intestinal transporters and increase the absorption of coadministered substrates, including HIV protease inhibitors and GS-7340 or tenofovir alafenamide hemifumarate, contributing to its effectiveness as a pharmacoenhancer.

As used herein, the term “coadminister” (or “coadministration”) refers to administration of two or more agents within a 24-hour period of each other, for example, as part of a clinical treatment regimen. In other embodiments, “coadminister” refers to administration of two or more agents within 2 hours of each other. In other embodiments, “coadminister” refers to administration of two or more agents within 30 minutes of each other. In other embodiments, “coadminister” refers to administration of two or more agents within 15 minutes of each other. In other embodiments, “coadminister” refers to administration of two or more agents at the same time, either as part of a single formulation or as multiple formulations that are administered by the same or different routes.

The term “unit dosage form” refers to a physically discrete unit, such as a capsule, tablet, or solution, that is suitable as a unitary dosage for a human patient, each unit containing a predetermined quantity of one or more active ingredient(s) calculated to produce a therapeutic effect, in association with at least one pharmaceutically acceptable diluent or carrier, or combination thereof. Unit dosage formulations contain a daily dose or unit daily subdose or an appropriate fraction thereof, of the active ingredient(s).

The term “subtherapeutic amount” of a compound is any amount of the compound that upon dosing is insufficient to achieve the desired therapeutic benefit.

The term “boosting amount” or “boosting dose” is the amount of a compound needed to improve the pharmacokinetics of a second compound (or increase availability or exposure). The boosting amount or boosting dose may improve the pharmacokinetics (or increase availability or exposure) of the second compound to a level that is therapeutic in a subject. In other words, a subtherapeutic amount of the second compound (i.e., subtherapeutic when administered without coadministration of the boosting amount) reaches a therapeutic level(s) in a subject due to improved pharmacokinetics (or increased availability or exposure) upon coadministration of the boosting amount.

The present invention also provides a method for the treatment or prophylaxis of diseases, disorders, and conditions. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV), human T-lymphotrophic virus (HTLV), rous sarcoma virus (RSV), and the avian leukosis virus. In general, three genes of the retrovirus genome code for the proteins of the mature virus: gag (group-specific antigen) gene, which codes for the core and structural proteins of the virus; pol (polymerase) gene, which codes for the enzymes of the virus, including reverse transcriptase, protease, and integrase; and env (envelope) gene, which codes for the retrovirus surface proteins.

Retroviruses attach to and invade a host cell by releasing a complex of RNA and the pol products, among other things, into the host cell. The reverse transcriptase then produces double-stranded DNA from the viral RNA. The double-stranded DNA is imported into the nucleus of the host cell and integrated into the host cell genome by the viral integrase. A nascent virus from the integrated DNA is formed when the integrated viral DNA is converted into mRNA by the host cell polymerase, and the proteins necessary for virus formation are produced by the action of the virus protease. The virus particle undergoes budding and is released from the host cell to form a mature virus.

The active agents may be administered to a human in any conventional manner. While it is possible for the active agents to be administered as raw compounds, they are preferably administered as a pharmaceutical composition. The salt, carrier, or diluent should be acceptable in the sense of being compatible with the other ingredients and not deleterious to the recipient thereof. Examples of carriers or diluents for oral administration include cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), and sodium lauryl sulfate.

The pharmaceutical compositions may be prepared by any suitable method, such as those methods well known in the art of pharmacy, for example, methods such as those described in Gennaro et al., Remington's Pharmaceutical Sciences (18th ed., Mack Publishing Co., 1990), especially Part 8: Pharmaceutical Preparations and their Manufacture. Such methods include the step of bringing into association GS-7340 or tenofovir alafenamide hemifumarate with the carrier or diluent and optionally one or more accessory ingredients. Such accessory ingredients include those conventional in the art, such as, fillers, binders, excipients, disintegrants, lubricants, colorants, flavoring agents, sweeteners, preservatives (e.g., antimicrobial preservatives), suspending agents, thickening agents, emulsifying agents, and/or wetting agents.

The term “GS-7340, or pharmaceutically acceptable salt thereof” or the like includes any amorphous, crystalline, co-crystalline, complex, or other physical form thereof. In one embodiment, a composition comprising a pharmaceutically acceptable coformer and GS-7340 is administered. The pharmaceutically acceptable coformer can be any pharmaceutically acceptable compound that is capable of forming a “pharmaceutically acceptable salt” with GS-7340. For example, the pharmaceutically acceptable coformer can be a pharmaceutically acceptable acid (e.g. adipic acid, L-aspartic acid, citric acid, fumaric acid, maleic acid, malic acid, malonic acid, succinic acid, tartaric acid, or oxalic acid). In one embodiment of the invention, the pharmaceutically acceptable coformer is a bis-acid. In another embodiment, the pharmaceutically acceptable coformer is fumaric acid. In another embodiment, a composition comprising a coformer and GS-7340 in a ratio of about 0.5±0.05 can be administered. One form of GS-7340 is a hemifumarate form (tenofovir alafenamide hemifumarate), as described further herein.

The pharmaceutical compositions may provide controlled, slow release or sustained release of the agents (e.g., GS-7340 or tenofovir alafenamide hemifumarate) over a period of time. The controlled, slow release or sustained release of the agents (e.g., GS-7340 or tenofovir alafenamide hemifumarate) may maintain the agents in the bloodstream of the human for a longer period of time than with conventional formulations. Pharmaceutical compositions include, but are not limited to, coated tablets, pellets, solutions, powders, capsules, and dispersions of GS-7340 or tenofovir alafenamide hemifumarate in a medium that is insoluble in physiologic fluids, or where the release of the therapeutic compound follows degradation of the pharmaceutical composition due to mechanical, chemical, or enzymatic activity.

The pharmaceutical compositions of the invention may be, for example, in the form of a pill, capsule, solution, powder, or tablet, each containing a predetermined amount of GS-7340 or tenofovir alafenamide hemifumarate. In an embodiment of the invention, the pharmaceutical composition is in the form of a tablet comprising GS-7340 or tenofovir alafenamide hemifumarate. In another embodiment of the invention, the pharmaceutical composition is in the form of a tablet comprising GS-7340 and the components of the tablet utilized and described in the Examples provided herein.

For oral administration, fine powders or granules may contain diluting, dispersing, and or surface active agents and may be present, for example, in water or in a syrup, in capsules or sachets in the dry state, or in a nonaqueous solution or suspension wherein suspending agents may be included, or in tablets wherein binders and lubricants may be included.

When administered in the form of a liquid solution or suspension, the formulation may contain GS-7340 or tenofovir alafenamide hemifumarate and purified water. Optional components in the liquid solution or suspension include suitable sweeteners, flavoring agents, preservatives (e.g., antimicrobial preservatives), buffering agents, solvents, and mixtures thereof. A component of the formulation may serve more than one function. For example, a suitable buffering agent also may act as a flavoring agent as well as a sweetener.

Suitable sweeteners include, for example, saccharin sodium, sucrose, and mannitol. A mixture of two or more sweeteners may be used. The sweetener or mixtures thereof are typically present in an amount of from about 0.001% to about 70% by weight of the total composition. Suitable flavoring agents may be present in the pharmaceutical composition to provide a cherry flavor, cotton candy flavor, or other suitable flavor to make the pharmaceutical composition easier for a human to ingest. The flavoring agent or mixtures thereof are typically present in an amount of about 0.0001% to about 5% by weight of the total composition.

Suitable preservatives include, for example, methylparaben, propylparaben, sodium benzoate, and benzalkonium chloride. A mixture of two or more preservatives may be used. The preservative or mixtures thereof are typically present in an amount of about 0.0001% to about 2% by weight of the total composition.

Suitable buffering agents include, for example, citric acid, sodium citrate, phosphoric acid, potassium phosphate, and various other acids and salts. A mixture of two or more buffering agents may be used. The buffering agent or mixtures thereof are typically present in an amount of about 0.001% to about 4% by weight of the total composition.

Suitable solvents for a liquid solution or suspension include, for example, sorbitol, glycerin, propylene glycol, and water. A mixture of two or more solvents may be used. The solvent or solvent system is typically present in an amount of about 1% to about 90% by weight of the total composition.

The pharmaceutical composition may be coadministered with adjuvants. For example, nonionic surfactants such as polyoxyethylene oleyl ether and n-hexadecyl polyethylene ether may be administered with or incorporated into the pharmaceutical composition to artificially increase the permeability of the intestinal walls. Enzymatic inhibitors may also be administered with or incorporated into the pharmaceutical composition.

### GS-7340

In one embodiment of the invention, a dose of 3 mg, 3±2 mg, or 3±1 mg of GS-7340, or a pharmaceutically acceptable salt thereof, is administered.

In one embodiment of the invention, a dose of 8±3 mg, 8±2 mg or 8±1 mg of GS-7340, or a pharmaceutically acceptable salt thereof, is administered.

In one embodiment of the invention, a unit dosage form comprises a dose of 8±2 mg of GS-7340, or a pharmaceutically acceptable salt thereof.

In various embodiments of the invention, a dose of 8±3 mg; 25±10 mg; 10±5 mg; 25±5 mg; 25±2 mg; 40±10 mg; 40±5 mg; 40±2 mg; 60±20 mg; 60±10 mg; 100±20 mg; 100±10 mg; 125±20 mg; 125±10 mg; 150±20 mg; 150±10 mg; 200±40 mg; or 200±15 mg of GS-7340, or a pharmaceutically acceptable salt thereof, is administered.

The desired daily dose of GS-7340 also may be administered as two, three, four, five, six, or more subdoses administered separately at appropriate intervals throughout the day, optionally, in unit dosage forms.

The concentration of tenofovir/GS-7340 in the bloodstream may be measured as the plasma concentration (e.g., ng/mL). Pharmacokinetic parameters for determining the plasma concentration include, but are not limited to, the maximum observed plasma concentration (Cₘₐₓ), observed plasma concentration at the end of the dosing interval or “trough” concentration (Cₜₐᵤ or Cₘᵢₙ), area under the plasma concentration time curve (AUC) from time zero up to the last quantifiable time point (AUC₀₋ₗₐₛₜ), AUC from time zero to infinity (AUC_{0-inf}), AUC over the dosing interval (AUCₜₐᵤ), time of maximum observed plasma concentration after administration (tₘₐₓ), and half-life of GS-7340 in plasma (t_{1/2}).

Administration of GS-7340 with food according to the methods of the invention may also increase absorption of GS-7340. Absorption of GS-7340 may be measured by the concentration attained in the bloodstream over time after administration of GS-7340. An increase in absorption by administration of GS-7340 with food may also be evidenced by an increase in Cₘₐₓ and/or AUC of GS-7340 as compared to the values if GS-7340 was administered without food. Typically protease inhibitors are administered with food.

### Tenofovir Alafenamide Hemifumarate

In one embodiment, there is provided a hemifumarate form of tenofovir alafenamide (i.e., tenofovir alafenamide hemifumarate). This form may have a ratio (i.e., a stoichiometric ratio or mole ratio) of fumaric acid to tenofovir alafenamide of 0.5±0.1, 0.5±0.05, 0.5±0.01, or about 0.5, or the like.

In one embodiment, tenofovir alafenamide hemifumarate consists of fumaric acid and tenofovir alafenamide in a ratio of 0.5±0.1.

In one embodiment, tenofovir alafenamide hemifumarate consists essentially of fumaric acid and tenofovir alafenamide in a ratio of 0.5±0.1.

In one embodiment, tenofovir alafenamide hemifumarate has an XRPD pattern comprising 2theta values of 6.9±0.2°, 8.6±0.2°, 10.0±0.2°, 11.0±0.2°, 12.2±0.2°, 15.9±0.2°, 16.3±0.2°, 20.2±0.2°, and 20.8±0.2°.

In one embodiment, tenofovir alafenamide hemifumarate has an XRPD pattern comprising at least four 2theta values selected from 6.9±0.2°, 8.6±0.2°, 10.0±0.2°, 11.0±0.2°, 12.2±0.2°, 15.9±0.2°, 16.3±0.2°, 20.2±0.2°, and 20.8±0.2°.

In one embodiment, tenofovir alafenamide hemifumarate has a DSC onset endotherm of 131±2° C., or 131±1° C.

In various embodiments, a tenofovir alafenamide hemifumarate composition comprises less than about 5%; 1%; or 0.5% by weight of tenofovir alafenamide monofumarate.

In one embodiment, a tenofovir alafenamide hemifumarate composition comprises no detectable tenofovir alafenamide monofumarate.

Tenofovir alafenamide (i.e., the compound 9-[(R)-2-[[(S)-[[(S)-1-(isopropoxycarbonyl)ethyl]amino]phenoxyphosphinyl]methoxy]propyl]adenine) can be prepared as described in U.S. Pat. No. 7,390,791.

In various embodiments of the invention, a dose of 3 mg; 3±2 mg; 3±1 mg; 8±3 mg; 8±2 mg; 8±1 mg;

In one embodiment of the invention, a unit dosage form comprises a dose of 8±2 mg of tenofovir alafenamide hemifumarate.

25±10 mg; 10±5 mg; 10 mg; 25±5 mg; 25±2 mg; 40±10 mg; 40±5 mg; 40±2 mg; 60±20 mg; 60±10 mg; 100±20 mg; 100±10 mg; 125±20 mg; 125±10 mg; 150±20 mg; 150±10 mg; 200±40 mg; or 200±15 mg of tenofovir alafenamide hemifumarate is administered.

The desired daily dose of tenofovir alafenamide hemifumarate also may be administered as two, three, four, five, six, or more subdoses administered separately at appropriate intervals throughout the day, optionally, in unit dosage forms.

The concentration of tenofovir, GS-7340, or tenofovir alafenamide hemifumarate in the bloodstream may be measured as the plasma concentration (e.g., ng/mL). Pharmacokinetic parameters for determining the plasma concentration include, but are not limited to, the maximum observed plasma concentration (Cₘₐₓ), observed plasma concentration at the end of the dosing interval or “trough” concentration (Cₜₐᵤ or Cₘᵢₙ), area under the plasma concentration time curve (AUC) from time zero up to the last quantifiable time point (AUC₀₋ₗₐₛₜ), AUC from time zero to infinity (AUC_{0-inf}), AUC over the dosing interval (AUCₜₐᵤ), time of maximum observed plasma concentration after administration (tₘₐₓ), and half-life of tenofovir, GS-7340, or tenofovir alafenamide hemifumarate in plasma (t_{1/2}).

Administration of GS-7340 or tenofovir alafenamide hemifumarate with food according to the methods of the invention may also increase absorption of GS-7340 or tenofovir alafenamide hemifumarate. Absorption of GS-7340 or tenofovir alafenamide hemifumarate may be measured by the concentration attained in the bloodstream over time after administration of GS-7340 or tenofovir alafenamide hemifumarate. An increase in absorption by administration of GS-7340 or tenofovir alafenamide hemifumarate with food may also be evidenced by an increase in Cₘₐₓ and/or AUC of GS-7340 or tenofovir alafenamide hemifumarate as compared to the values if GS-7340 or tenofovir alafenamide hemifumarate was administered without food. Typically protease inhibitors are administered with food.

### Selective Crystallization—Tenofovir Alafenamide Hemifumarate

In one embodiment, tenofovir alafenamide hemifumarate can be prepared using selective crystallization. An example of a scheme for this preparation method is as follows.

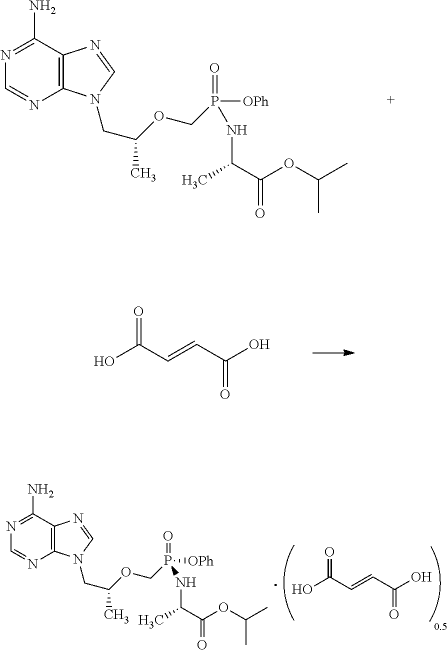

The method can be carried out by subjecting a solution comprising: a) a suitable solvent; b) fumaric acid; c) tenofovir alafenamide; and, optionally, d) one or more seeds comprising tenofovir alafenamide hemifumarate, to conditions that provide for the crystallization of fumaric acid and tenofovir alafenamide. The starting solution can contain the single diastereomer of tenofovir alafenamide or a mixture of tenofovir alafenamide and one or more of its other diastereomers (e.g., GS-7339, as described in U.S. Pat. No. 7,390,791).

The selective crystallization can be carried out in any suitable solvent. For example, it can be carried out in a protic solvent or in an aprotic organic solvent, or in a mixture thereof. In one embodiment, the solvent comprises a protic solvent (e.g., water or isopropyl alcohol). In another embodiment, the solvent comprises an aprotic organic solvent (e.g., acetone, acetonitrile (ACN), toluene, ethyl acetate, isopropyl acetate, heptane, tetrahydrofuran (THF), 2-methyl THF, methyl ethyl ketone, or methyl isobutyl ketone, or a mixture thereof). In one embodiment, the solvent comprises ACN or a mixture of ACN and up to about 50% methylene chloride (by volume). The selective crystallization also can be carried out at any suitable temperature, for example, a temperature in the range of from about 0° C. to about 70° C. In one specific embodiment, the resolution is carried out at a temperature of about 0° C.

One major advantage of the hemifumarate form of tenofovir alafenamide over the monofumarate form is its exceptional capability to purge GS-7339 (i.e., 9-[(R)-2-[[(R)-[[(S)-1-(isopropoxycarbonyl)ethyl]amino]phenoxyphosphinyl]methoxy]propyl]adenine; described in, e.g., U.S. Pat. No. 7,390,791), which is the major diastereomeric impurity in the active pharmaceutical ingredient. Thus, the hemifumarate form of tenofovir alafenamide can be more readily and easily separated from impurities than the monofumarate form. Other major advantages of tenofovir alafenamide hemifumarate over the monofumarate form include improved thermodynamic and chemical stability (including long-term storage stability), superior process reproducibility, superior drug product content uniformity, and a higher melting point.

Tenofovir alafenamide hemifumarate is useful in the treatment and/or prophylaxis of one or more viral infections in man or animals, including infections caused by DNA viruses. RNA viruses, herpesviruses (e.g., CMV, HSV 1, HSV 2, VZV), retroviruses, hepadnaviruses (e.g., HBV), papillomavirus, hantavirus, adenoviruses and HIV. U.S. Pat. No. 6,043,230 (incorporated by reference herein in its entirety) and other publications describe the anti-viral specificity of nucleotide analogs, such as tenofovir disoproxil. Like tenofovir disoproxil, tenofovir alafenamide is another prodrug form of tenofovir, and can be used in the treatment and/or prophylaxis of the same conditions.

Tenofovir alafenamide hemifumarate can be administered by any route appropriate to the condition to be treated. Suitable routes include oral, rectal, nasal, topical (including ocular, buccal, and sublingual), vaginal, and parenteral (including subcutaneous, intramuscular, intravenous, intradermal, intrathecal, and epidural). Generally, tenofovir alafenamide hemifumarate is administered orally, but it can be administered by any of the other routes noted herein.

Accordingly, pharmaceutical compositions include those suitable for topical or systemic administration, including oral, rectal, nasal, buccal, sublingual, vaginal, or parenteral (including subcutaneous, intramuscular, intravenous, intradermal, intrathecal, and epidural) administration. The formulations are in unit dosage form and are prepared by any of the methods well known in the art of pharmacy.

For oral therapeutic administration, the tenofovir alafenamide hemifumarate may be combined with one or more excipients and used in the form of ingestible tablets, buccal tablets, troches, capsules, elixirs, suspensions, syrups, wafers, and the like. Such pharmaceutical compositions and preparations will typically contain at least 0.1% of tenofovir alafenamide hemifumarate. The percentage of this active compound in the compositions and preparations may, of course, be varied and may conveniently be between about 2% to about 60% or more of the weight of a given unit dosage form. The amount of active compound in such therapeutically useful pharmaceutical compositions is preferably such that an effective dosage level will be obtained upon administration of a single-unit dosage (e.g., tablet). Other dosage formulations may provide therapeutically effective amounts of tenofovir alafenamide hemifumarate upon repeated administration of subclinically effective amounts of the same. Preferred unit dosage formulations include those containing a daily dose (e.g., a single daily dose), as well as those containing a unit daily subclinical dose, or an appropriate fraction thereof (e.g., multiple daily doses), of tenofovir alafenamide hemifumarate.

Pharmaceutical compositions suitable for oral administration may be presented as discrete units such as capsules, cachets, or tablets, each containing a predetermined amount of tenofovir alafenamide hemifumarate; as a powder or granules; as a solution or a suspension in an aqueous liquid or a nonaqueous liquid; or as an oil-in-water liquid emulsion or a water-in-oil liquid emulsion. Tenofovir alafenamide hemifumarate may also be presented as a bolus, electuary, or paste.

Tenofovir alafenamide hemifumarate is preferably administered as part of a pharmaceutical composition or formulation. Such pharmaceutical composition or formulation comprises tenofovir alafenamide hemifumarate together with one or more pharmaceutically acceptable carriers/excipients, and optionally other therapeutic ingredients. The excipient(s)/carrier(s) must be “acceptable” in the sense of being compatible with the other ingredients of the formulation and not deleterious to the patient. Excipients include, but are not limited to, substances that can serve as a vehicle or medium for tenofovir alafenamide hemifumarate (e.g., a diluent carrier). They may be enclosed in hard or soft shell gelatin capsules, may be compressed into tablets, or may be incorporated directly with the food of the patient's diet.

Accordingly, the tablets, troches, pills, capsules, and the like may also contain, without limitation, the following: a binder(s), such as hydroxypropyl cellulose, povidone, or hydroxypropyl methylcellulose; a filler(s), such as microcrystalline cellulose, pregelatinized starch, starch, mannitol, or lactose monohydrate; a disintegrating agent(s), such as croscarmellose sodium, cross-linked povidone, or sodium starch glycolate; a lubricant(s), such as magnesium stearate, stearic acid, or other metallic stearates; a sweetening agent(s), such as sucrose, fructose, lactose, or aspartame; and/or a flavoring agent(s), such as peppermint, oil of wintergreen, or a cherry flavoring. When the unit dosage form is a capsule, it may contain, in addition to materials of the above types, a liquid carrier, such as a vegetable oil or a polyethylene glycol. Various other materials may be present as coatings or to otherwise modify the physical form of the solid unit dosage form. For instance, tablets, pills, or capsules may be coated with gelatin, polymers, wax, shellac, or sugar and the like. Of course, any material used in preparing any unit dosage form typically will be pharmaceutically acceptable and substantially nontoxic in the amounts employed. In addition, tenofovir alafenamide hemifumarate may be incorporated into sustained-release preparations and devices.

For infections of the eye or other external tissues, e.g., mouth and skin, the pharmaceutical compositions are preferably applied as a topical ointment or cream containing tenofovir alafenamide hemifumarate in an amount of, for example, 0.01 to 10% w/w (including active ingredient in a range between 0.1% and 5% in increments of 0.1% w/w such as 0.6% w/w, 0.7% w/w, etc.), preferably 0.2 to 3% w/w and most preferably 0.5 to 2% w/w. When formulated in an ointment, the active ingredient may be employed with either a paraffinic or a water-miscible ointment base. Alternatively, the active ingredient may be formulated in a cream with an oil-in-water cream base.

Pharmaceutical compositions suitable for topical administration in the mouth include lozenges comprising tenofovir alafenamide hemifumarate in a flavored basis, for example, sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising, for example, cocoa butter or a salicylate.

Pharmaceutical formulations suitable for parenteral administration are sterile and include aqueous and nonaqueous injection solutions that may contain antioxidants, buffers, bacteriostats, and solutes that render the formulation isotonic with the blood of the intended recipient; and aqueous and nonaqueous sterile suspensions that may include suspending agents and thickening agents. The formulations may be presented in unit-dose or multi-dose containers, for example, sealed ampoules and vials with elastomeric stoppers, and may be stored in a freeze-dried (lyophilized) condition requiring only the addition of the sterile liquid carrier (e.g., water for injections) immediately prior to use. Injection solutions and suspensions may be prepared from sterile powders, granules, and tablets of the kind previously described.

In addition to the ingredients particularly mentioned above, the pharmaceutical compositions/formulations may include other ingredients conventional in the art, having regard to the type of formulation in question.

In another embodiment, there is provided veterinary compositions comprising tenofovir alafenamide hemifumarate together with a veterinary carrier therefor. Veterinary carriers are materials useful for the purpose of administering the composition to cats, dogs, horses, rabbits, and other animals, and may be solid, liquid, or gaseous materials that are otherwise inert or acceptable in the veterinary art and are compatible with the active ingredient. These veterinary compositions may be administered orally, parenterally, or by any other desired route.

The tenofovir alafenamide hemifumarate can be used to provide controlled release pharmaceutical formulations containing a matrix or absorbent material and an active ingredient of the invention, in which the release of the active ingredient can be controlled and regulated to allow less frequent dosing or to improve the pharmacokinetic or toxicity profile of the compound. Controlled release formulations adapted for oral administration, in which discrete units comprising a compounds of the invention, can be prepared according to conventional methods.

Useful dosages of tenofovir alafenamide hemifumarate can be determined by comparing in vitro activities, and the in vivo activities in animal models. Methods for the extrapolation of effective amounts/dosages in mice and other animals to therapeutically effective amounts/dosages in humans are known in the art.

The amount of tenofovir alafenamide hemifumarate required for use in treatment will vary with several factors, including but not limited to the route of administration, the nature of the condition being treated, and the age and condition of the patient; ultimately, the amount administered will be at the discretion of the attendant physician or clinician. The therapeutically effective amount/dose of tenofovir alafenamide hemifumarate depends, at least, on the nature of the condition being treated, any toxicity or drug interaction issues, whether the compound is being used prophylactically (e.g., sometimes requiring lower doses) or against an active disease or condition, the method of delivery, and the pharmaceutical formulation, and will be determined by the clinician using conventional dose escalation studies.

In one embodiment, the oral dose of tenofovir alafenamide hemifumarate may be in the range from about 0.0001 to about 100 mg/kg body weight per day, for example, from about 0.01 to about 10 mg/kg body weight per day, from about 0.01 to about 5 mg/kg body weight per day, from about 0.5 to about 50 mg/kg body weight per day, from about 1 to about 30 mg/kg body weight per day, from about 1.5 to about 10 mg/kg body weight per day, or from about 0.05 to about 0.5 mg/kg body weight per day. As a nonlimiting example, the daily candidate dose for an adult human of about 70 kg body weight will range from about 0.1 mg to about 1000 mg, or from about 1 mg to about 1000 mg, or from about 5 mg to about 500 mg, or from about 1 mg to about 150 mg, or from about 5 mg to about 150 mg, or from about 5 mg to about 100 mg, or about 10 mg, and may take the form of single or multiple doses. In one embodiment, the oral dose of tenofovir alafenamide hemifumarate may be in the form of a combination of agents (e.g., tenofovir alafenamide hemifumarate/emtricitabine/elvitegravir/cobicistat).

The pharmaceutical compositions described herein may further include one or more therapeutic agents in addition to tenofovir alafenamide hemifumarate. In one specific embodiment of the invention, the additional therapeutic agent can be selected from the group consisting of HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

Therapeutic methods include administering tenofovir alafenamide hemifumarate to a subject/patient in need of the same as a therapeutic or preventative treatment. Thus, tenofovir alafenamide hemifumarate may be administered to a subject/patient having a medical disorder or to a subject who may acquire the disorder. One of ordinary skill will appreciate that such treatment is given in order to ameliorate, prevent, delay, cure, and/or reduce the severity of a symptom or set of symptoms of a disorder (including a recurring disorder). The treatment may also be given to prolong the survival of a subject, e.g., beyond the survival time expected in the absence of such treatment. The medical disorders that may be treated with tenofovir alafenamide hemifumarate include those discussed herein, including without limitation, HIV infection (including, without limitation, HIV-1 and HIV-2 infections; preferably HIV-1 infection) and HBV infection.

### Formulation of Cobicistat

When cobicistat or a pharmaceutically acceptable salt thereof is combined with certain specific solid carrier particles (e.g. silica derivatives), the resulting combination possesses improved physical properties. Even though cobicistat is hygroscopic in nature, the resulting combination has comparatively low hygroscopicity. Additionally, the resulting combination is a free-flowing powder, with high loading values for cobicistat, acceptable physical and chemical stability, rapid drug release properties, and excellent compressibility. Thus, the resulting combination can readily be processed into solid dosage forms (e.g. tablets), which possess good drug release properties, low tablet friability, good chemical and physical stability, and a low amount of residual solvents. The compositions of the invention represent a significant advance that facilitates the commercial development of cobicistat for use in treating viral infections such as HIV.

Cobicistat can be combined with any suitable solid carrier, provided the resulting combination has physical properties that allow it to be more easily formulated than the parent compound. For example, suitable solid carriers include kaolin, bentonite, hectorite, colloidal magnesium-aluminum silicate, silicon dioxide, magnesium trisilicate, aluminum hydroxide, magnesium hydroxide, magnesium oxide and talc. In one embodiment of the invention, the solid carrier can comprise calcium silicate (such as ZEOPHARM), or magnesium aluminometasilicate (such as NEUSILIN). As used herein, “loaded” on a solid carrier includes, but is not limited to a compound being coated in the pores and on the surface of a solid carrier.

Suitable silica derivatives for use in the compositions of the invention and methods for preparing such silica derivatives include those that are described in international patent application publication number WO 03/037379 and the references cited therein. A specific silica material that is particularly useful in the compositions and methods of the invention is AEROPERL® 300 (fumed silica), which is available from Evonik Degussa AG, Dusseldorf, Germany. Other materials having physical and chemical properties similar to the silica materials described herein can also be used.

### Ritonavir

Ritonavir (1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) was developed as an inhibitor of retroviral (HIV) protease; however, it is now used in a manner similar to cobicistat to inhibit the action of certain cytochrome P450 proteases (specifically Cyp3A4) thereby allowing greater circulating levels of drugs for treatment of HIV than would be obtained by administration of the drugs alone. Although none of GS-7340, tenofovir, or tenofovir alafenamide hemifumarate apparently is metabolized by cytochrome P450 proteases, it is contemplated that ritonavir may be used in the manner that cobicistat is used to boost the circulating levels of GS-7340, tenofovir, or tenofovir alafenamide hemifumarate, to improve the pharmacokinetics of GS-7340, tenofovir, or tenofovir alafenamide hemifumarate and achieve the other advantages of the use of cobicistat as disclosed herein.

### Combination Treatment

The compounds and methods of the invention may also be used with any of the following compounds:

1) amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, GS-8374, PPL-100, DG35, and AG 1859;

2) an HIV nonnucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirine), BILR 355 BS, VRX 840773, UK-453061, and RDEA806;

3) an HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (±-emtricitabine), D-d4FC, phosphazide, fozivudine tidoxil, apricitibine (AVX754), GS-7340, KP-1461, and fosalvudine tidoxil (formerly HDP 99.0003);

4) an HIV nucleotide inhibitor of reverse transcriptase, e.g., tenofovir disoproxil fumarate and adefovir dipivoxil;

5) an HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), elvitegravir, BMS-538158, GSK364735C, BMS-707035, MK-2048, and BA 011;

6) a gp41 inhibitor, e.g., enfuvirtide, sifuvirtide, FB006M, and TRI-1144;

7) a CXCR4 inhibitor, e.g., AMD-070;

8) an entry inhibitor, e.g., SP01A;

9) a gp120 inhibitor, e.g., BMS-488043 or BlockAide/CR;

10) a G6PD and NADH-oxidase inhibitor, e.g., immunitin;

11) a CCR5 inhibitor, e.g., aplaviroc, vicriviroc, maraviroc, PRO-140, INCB15050, PF-232798 (Pfizer), and CCR5 mAb004;

12) other drugs for treating HIV, e.g., BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDX010 (ipilimumab), PBS 119, ALG 889, and PA-1050040 (PA-040);

13) an interferon, e.g., pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen+actimmune, IFN-omega with DUROS, albuferon, locteron, Albuferon, Rebif, oral interferon alpha, IFNalpha-2b XL, AVI-005, PEG-Infergen, and pegylated IFN-beta;

14) a ribavirin analog, e.g., rebetol, copegus, viramidine (taribavirin);

15) an NS5b polymerase inhibitor, e.g., NM-283, valopicitabine, R1626, PSI-6130 (R1656), HCV-796, BILB 1941, XTL-2125, MK-0608, NM-107, R7128 (R4048), VCH-759, PF-868554, and GSK625433;

16) an NS3 protease inhibitor, e.g., SCH-503034 (SCH-7), VX-950 (telaprevir), BILN-2065, BMS-605339, and ITMN-191;

17) an alpha-glucosidase 1 inhibitor, e.g., MX-3253 (celgosivir), UT-231B;

18) hepatoprotectants, e.g., IDN-6556, ME 3738, LB-84451, and MitoQ;

19) a nonnucleoside inhibitor of HCV, e.g., benzimidazole derivatives, benzo-1,2,4-thiadiazine derivatives, phenylalanine derivatives, A-831, GS-9190, and A-689; and

20) other drugs for treating HCV, e.g., zadaxin, nitazoxanide (alinea), BIVN-401 (virostat), PYN-17 (altirex), KPE02003002, actilon (CPG-10101), KRN-7000, civacir, GI-5005, ANA-975, XTL-6865, ANA 971, NOV-205, tarvacin, EHC-18, NIM811, DEBIO-025, VGX-410C, EMZ-702, AVI 4065, Bavituximab, Oglufanide, and VX-497 (merimepodib).

Exemplary combinations (including, but not limited to, single tablet regimens) include (a) emtricitabine/darunavir/cobicistat/GS-7340; (b) emtricitabine/darunavir/cobicistat/tenofovir alafenamide hemifumarate; (c) emtricitabine/darunavir/cobicistat/tenofovir disoproxil fumarate (TDF); (d) emtricitabine/elvitegravir/cobicistat/GS-7340; (e) emtricitabine/elvitegravir/cobicistat/tenofovir alafenamide hemifumarate; (f) emtricitabine/elvitegravir/cobicistat/TDF; (g) cobicistat/GS-7340; (h) cobicistat/tenofovir alafenamide hemifumarate; and (i) cobicistat/TDF. The combinations listed above may contain various dosages of the component agents; as nonlimiting examples, combination (b) above can include 200 mg of emtricitabine, 800 mg of darunavir, 150 mg of cobicistat, and 10 mg of tenofovir alafenamide hemifumarate, and combination (e) above can include 200 mg of emtricitabine, 150 mg of elvitegravir, 150 mg of cobicistat, and 10 mg of tenofovir alafenamide hemifumarate.

An alternative exemplary combination is emtricitabine and tenofovir alafenamide hemifumarate. The combination of emtricitabine and TDF is currently marketed as TRUVADA®. See also U.S. Patent Application Publication No. 2004/0224916, the content of which is hereby incorporated by reference herein in its entirety. The present invention provides the combination of emtricitabine and tenofovir alafenamide hemifumarate. This combination may contain various dosages of the two component agents; as a nonlimiting example, this combination can include 200 mg of emtricitabine and 10 mg of tenofovir alafenamide hemifumarate.

An additional alternative exemplary combination is emtricitabine, rilpivirine, and tenofovir alafenamide hemifumarate. The combination of emtricitabine, rilpivirine (a nonnucleoside reverse transcriptase inhibitor), and TDF is currently marketed as COMPLERA®. The present invention provides the combination of emtricitabine, rilpivirine, and tenofovir alafenamide hemifumarate. This combination may contain various dosages of the three component agents; as a nonlimiting example, this combination can include 200 mg of emtricitabine, 25 mg of rilpivirine, and 10 mg of tenofovir alafenamide hemifumarate.

A further additional alternative exemplary combination is GS-9441 and tenofovir alafenamide hemifumarate. The combination of GS-9441 (a reverse transcriptase inhibitor) and GS-7340 is disclosed in U.S. Patent Application Publication No. 2009/0075939 and U.S. Pat. No. 8,354,421, the content of each of which is hereby incorporated by reference herein in its entirety. The present invention provides the combination of GS-9441 and tenofovir alafenamide hemifumarate. This combination may contain various dosages of the two component agents; as a nonlimiting example, this combination can include 5-1500 mg of GS-9441 and 10 mg of tenofovir alafenamide hemifumarate.

Exemplary amounts of agents in various combinations include, but are not limited to, the following: (1) cobicistat: 10-500 mg, 50-500 mg, 75-300 mg, 100-200 mg, or 150 mg; (2) tenofovir alafenamide hemifumarate: 1-60 mg, 3-40 mg, 5-30 mg, 8-20 mg, or 10 mg; (3) emtricitabine: 10-500 mg, 50-500 mg, 75-300 mg, 150-250 mg, or 200 mg; (4) elvitegravir: 10-500 mg, 50-500 mg, 75-300 mg, 100-200 mg, or 150 mg; (5) darunavir: 300-1800 mg, 400-1600 mg, 500-1200 mg, 600-1000 mg, or 800 mg; and (6) rilpivirine: 5-100 mg, 10-80 mg, 15-60 mg, 20-40 mg, or 25 mg. One of skill in the art will know that, in the case of administering a pharmaceutically acceptable salt or complex of an agent, the amount administered will be adjusted relative to the weight of the component added to produce the salt or complex.

The invention will now be illustrated by the following nonlimiting Examples. The Synthetic Examples provided herein describe the synthesis of compounds of the invention as well as intermediates used to prepare compounds of the invention.

### Synthetic Examples

### Synthetic Example 1

### Preparation of Diastereomeric Mixture of 9-[(R)-2-[[(R,S)-1-[[(S)-(isopropoxycarbonyl)ethyl]amino]phenoxyphosphinyl]methoxy]propyl]adenine (15)

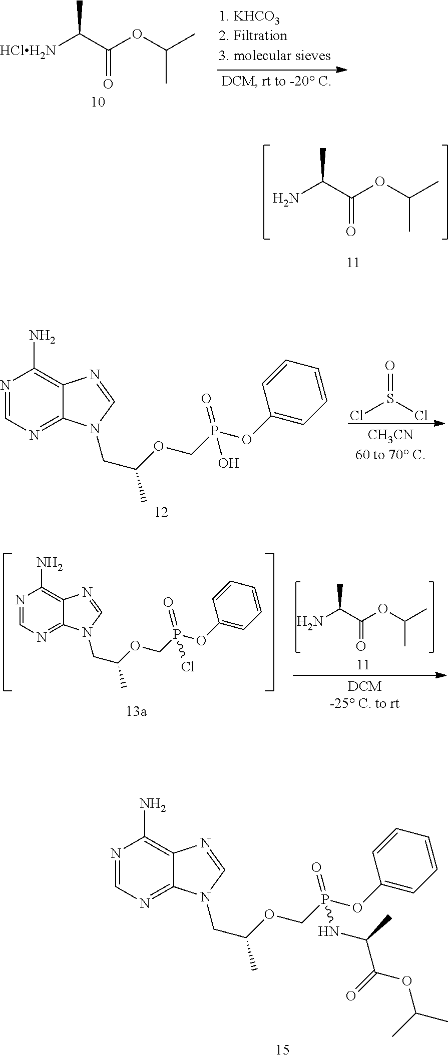

### a. Preparation of Compound 11

Isopropyl L-alanine ester hydrochloride 10 (1 kg, 5.97 mol, 1.0 equiv) and potassium bicarbonate (1.45 kg, 14.5 mol, 2.43 equiv) were agitated in DCM (4 kg) for 10-14 hours with maximum agitation, maintaining the pot temperature between 19 and 25° C. The mixture was then filtered and rinsed forward with DCM (2 kg). The filtrate was dried over a bed of 4 Å molecular sieves until the water content of the solution was ≦0.05%. The resultant stock solution containing compound 11 was then cooled to a pot temperature of −20° C. and held for further use.

### b. Preparation of Compound 13a

To a solution of thionyl chloride (0.72 kg, 6.02 mol, 2.19 equiv) in acetonitrile (5.5 kg) at 60° C. was added compound 12 (1 kg, 2.75 mol, 1.00 equiv) in 10 equal portions over 2 hours. The pot temperature was then adjusted to 70° C. and stirred for 1-3 hours until deemed complete by ³¹P NMR analysis (Target: >97.0% conversion of starting material signal at 12.6 ppm to product signal at 22.0 ppm). The pot temperature was then adjusted to 40° C. and vacuum applied. The mixture was distilled to dryness, maintaining a maximum jacket temperature of 40° C. The dry residue was then taken up in dichloromethane (30 kg) and the pot temperature adjusted to 19-25° C. The resultant slurry containing compound 13a was held for further use.

### c. Preparation of Compound 15

To the stock solution of isopropyl L-alanine ester 11 (4.82 equiv) at −25° C. was added slurry containing compound 13a (1.0 equiv) over a minimum of 2 hours, maintaining the pot temperature≦−10° C. The mixture was then held at a temperature ≦−10° C. for at least 30 minutes, then the pH checked using water wet pH paper. If the pH was <4, adjustment with triethylamine to pH 4-7 was performed. The pot temperature was then adjusted to room temperature (19-25° C.). In a separate vessel, a solution of sodium phosphate monobasic (2.2 kg, 18 mol, 6.90 equiv) in water (16 kg) was prepared. Half of the sodium phosphate monobasic solution was charged to the phosphonamidate reactor, and vigorously stirred. The layers were settled and partitioned. The organic layer was washed again with the remaining half of sodium phosphate monobasic solution. In a separate vessel, a solution of potassium bicarbonate (1.1 kg, 11 mol, 4.22 equiv) in water (5.5 kg) was prepared. Half of the potassium bicarbonate solution was charged to the organic phase, and vigorously stirred. The layers were settled and partitioned. The organic layer was washed again with the remaining half of the potassium bicarbonate solution followed by a final water (3.3 kg) wash. The organic phase was then retained and distilled to a volume of ca. 6 L. The resultant solution was analyzed for water content. If the water content was >1.0%, DCM could be charged and the distillation to ca. 6 L repeated. When the solution water content was less than or about 1.0%, the pot temperature was adjusted to 19-25° C. prior to discharge of the stock solution in DCM to provide the diastereomeric mixture of 9-[(R)-2-[[(R,S)-1-[[(S)-(isopropoxycarbonyl)ethyl]amino]phenoxyphosphinyl]methoxy]propyl]adenine (15). ¹H NMR (400 MHz, CDCl₃): δ 1.20-1.33 (m, 12H), 3.62-3.74 (m, 1H), 3.86-4.22 (m, 5H), 4.30-4.44 (m, 1H), 4.83-5.10 (m, 1H), 6.02 (br s, 3H), 7.18-7.34 (m, 5H), 7.98-8.02 (m, 1H), 8.32-8.36 (m, 1H); ³¹P NMR (162 MHz, CDCl₃): δ. 21.5, 22.9.

### Synthetic Example 2

### Crystallization-Induced Dynamic Resolution of Diastereomeric Mixture of 9-[(R)-2-[[(R,S)-1-[[(S)-(isopropoxycarbonyl)ethyl]amino]phenoxyphosphinyl]methoxy]propyl]adenine (15) to provide 9-[(R)-2-[[(S)-[[(S)-1-(isopropoxycarbonyl)ethyl]amino]phenoxyphosphinyl]methoxy]propyl]adenine (16)

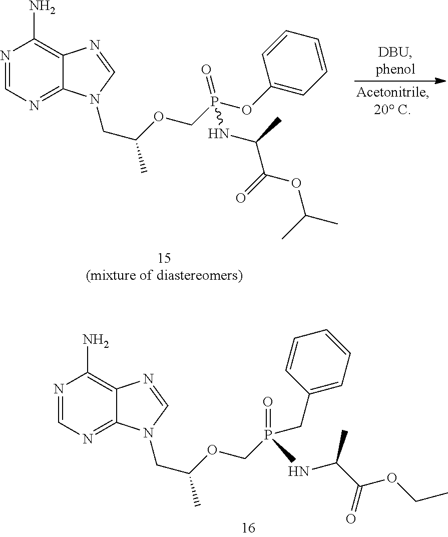 [SMILES: CC(C)OC(=O)[C@H](C)NP(=O)(CO[C@H](C)Cn1cnc2c(N)ncnc21)Oc1ccccc1 ; CCOC(=O)[C@H](C)N[P@](=O)(CO[C@H](C)Cn1cnc2c(N)ncnc21)Cc1ccccc1]

A 22 wt % solution of diastereomeric mixture of 9-[(R)-2-[[(R,S)-1-[[(S)-(isopropoxycarbonyl)ethyl]amino]phenoxyphosphinyl]methoxy]propyl]adenine (15) in acetonitrile (2.3 kg solution, 0.51 kg 15, 1.1 mol, 1 equiv) was charged to a vessel equipped with an overhead stirrer, distillation apparatus, and nitrogen inlet. The mixture was concentrated by distillation at 100-300 mbar over a temperature range of 45-55° C. to a final concentration of 30-35 wt %. The distillation apparatus was then removed and the solution was cooled to 20° C. The solution was seeded with 2.0% compound 16 and allowed to stir for one hour at 20° C. Phenol (9.9 g, 0.11 mol, 0.1 equiv) and DBU (16 g, 0.11 mol, 0.1 equiv) were added and the mixture was stirred for an additional 24 hours or until the weight percent of compound 16 remaining in solution was less than 12%. The slurry was then cooled to 0° C. and stirred for an additional 18 hours at 0° C. The slurry was filtered and washed with a 1:1 solution of isopropyl acetate:acetonitrile (1.5 L) at 0° C. The solids were dried in a vacuum oven at 50° C. to give 0.40 kg of compound 16 (80% yield) as a white solid. ¹H NMR (400 MHz, CDCl₃): δ 1.21 (m, 9H), 1.28 (d, J=7.0 Hz, 3H), 3.65 (dd, J=13.1, 10.7, 1H) 4.00 (m, 4H), 4.33 (dd, J=14.4, 3.1 Hz, 1H), 5.00 (m, 1H) 6.00 (bs, 2H), 6.99 (m, 2H), 7.07 (m, 1H), 7.19 (m, 2H), 7.97 (s, 1H), 8.33 (s, 1H). ³¹P NMR (162 MHz, CDCl₃): δ. 20.8.

### Synthetic Example 3

### Preparation of Compound 13a in High Diastereomeric Purity

To a slurry of compound 12 (10.0 g, 27.5 mmol, 1.00 equiv) in toluene (60 mL) at ambient temperature was added thionyl chloride (3.0 mL, 41 mmol, 1.5 equiv). The slurry was heated to 70° C. and agitated for 48-96 hours until reaction and diastereomeric enrichment were deemed complete by HPLC (Target: >97.0% conversion of compound 12 to compound 13a and >90:10 diastereomeric ratio of compound 13a). The mixture was concentrated to dryness by vacuum distillation, and the dry residue was taken up in toluene (50 mL). The resultant slurry containing compound 13a was held at ambient temperature for further use.

### Synthetic Example 4

### Preparation of 9-[(R)-2-[[(R,S)-1-[[(S)-(isopropoxycarbonyl)ethyl]amino]phenoxyphosphinyl]methoxy]propyl]adenine (15) in High Diastereomeric Purity

To a solution of isopropyl L-alanine ester 11 (4.50 equiv) in DCM (80 mL) at −25° C. was added a slurry containing compound 13a (1.00 equiv) that is at least 90% diastereomerically pure in toluene (50 mL) over a minimum of 45 minutes, maintaining the internal temperature ≦−20° C. The mixture was then held at a temperature ≦−20° C. for at least 30 minutes, and the pH checked using water wet pH paper. If the pH was <4, it was adjusted with triethylamine to pH 4-7. The pot temperature was adjusted to room temperature (19-25° C.). The mixture was transferred to a separatory funnel and washed sequentially with 10% w/v aqueous solution of sodium phosphate monobasic (2×50 mL), 15% w/v aqueous solution of potassium bicarbonate (2×20 mL), and water (50 mL). The final organic layer was dried over anhydrous sodium sulfate, filtered, and concentrated in vacuo to a viscous amber oil. The oil was dissolved in toluene/acetonitrile (4:1) (50 mL), and the solution was seeded with 9-[(R)-2-[[(R,S)-1-[[(S)-(isopropoxycarbonyl)ethyl]amino]phenoxyphosphinyl]methoxy]propyl]adenine (about 1 mg, 99:1 diastereomeric ratio) and stirred for 2 hours at ambient temperature. The resultant slurry was filtered and the filter cake was washed with toluene/acetonitrile (4:1) (15 mL) and dried in a vacuum oven at 40° C. for 16 hours to give the product, 9-[(R)-2-[[(R,S)-1-[[(S)-(isopropoxycarbonyl)ethyl]amino]phenoxyphosphinyl]methoxy]propyl]adenine (15), as a white solid (10.0 g, 76.4%, 97.5:2.5 diastereomeric ratio). ¹H NMR (400 MHz, CDCl₃): δ 1.20-1.33 (m, 12H), 3.62-3.74 (m, 1H), 3.86-4.22 (m, 5H), 4.30-4.44 (m, 1H), 4.83-5.10 (m, 1H), 6.02 (br s, 3H), 7.18-7.34 (m, 5H), 7.98-8.02 (m, 1H), 8.32-8.36 (m, 1H); ³¹P NMR (162 MHz, CDCl₃): δ. 21.5, 22.9.

### Synthetic Example 5

### Preparation of Compound 12

PMPA (100.0 g, 0.35 mol, 1 equiv) was charged to a vessel equipped with an overhead stirrer, reflux condenser and nitrogen inlet followed by acetonitrile (800 mL). To the vessel was added triethylamine (71.0 g, 0.70 mol, 2 equiv) followed by DMAP (42.6 g, 0.35 mol, 1 equiv) and triphenylphosphite (162.1 g, 0.52 mol, 1.5 equiv). The mixture was heated to 80° C. and agitated for ≧48 hours at 80° C. or until the reaction was complete by ³¹P NMR. (A sample directly from the reaction is taken and an insert containing 10% H₃PO₂ in D₂O is added. The intermediate formed is the PMPA anhydride and is at 6 ppm; the product is at 11 ppm. The reaction is deemed complete when less than 5% anhydride is present). The reaction mixture was distilled to ˜1.5 volumes of acetonitrile and diluted with ethyl acetate (200 mL) and water (300 mL). The aqueous layer was separated and washed with ethyl acetate (200 mL) twice. The aqueous layer was recharged to the vessel and pH adjusted to pH 3 using 12.1 M HCl (21.0 mL). The reaction was then seeded with 0.05% of compound 12 seed and allowed to stir at 25° C. Additional 12.1 M HCl was added over 20 minutes (7.0 mL) until pH 2 was achieved. The crystallization was allowed to stir at ambient temperature for 30 minutes and then cooled to 10° C. over 2 hours. Once at 10° C. the crystallization was allowed to stir for 2.5 hours at 10° C. The slurry was filtered and washed with pH 1.5 water (200 g). After drying in the vacuum oven, 102.2 g of compound 12 (81% yield) was obtained as a white solid. ¹H NMR (400 MHz, D₂O): δ 1.31 (d, J=6.1 Hz, 3H), 3.59 (dd, J=14.0, 9.0 Hz, 1H), 3.85 (dd, J=14.0, 9.0 Hz, 1H), 4.1 (m, 1H), 4.3 (dd, J=15.0, 9.0 Hz, 1H), 4.5 (dd, J=15.0, 2 Hz, 1H), 6.75 (d, J=7 Hz, 2H), 7.15 (t, J=7 Hz, 1H), 7.25 (t, J=7 Hz, 2H), 8.26 (s, 1H), 8.35 (s, 1H). ³¹P NMR (162 MHz, D₂O): δ. 14.8.

### Synthetic Examples

### Tenofovir Alafenamide Hemifumarate

### Synthetic Example 6

Tenofovir alafenamide monofumarate solids (5.0 g) and 9-[(R)-2-[[(R)-[[(S)-1-(isopropoxycarbonyl)ethyl]amino]phenoxyphosphinyl]methoxy]propyl]adenine (GS-7339) monofumarate solids (0.75 g) were charged into 35 g MTBE at 22° C. and the mixture was stirred for 1 hour. A slurry was formed and was dried in a rotary evaporator. 58 g acetonitrile (ACN) was charged into the solids and the mixture was heated to reflux to dissolve the solids. The resulting solution was allowed to cool naturally while agitated. A slurry was formed, and the slurry was further cooled by an ice-water bath. The solids were isolated by filtration and washed with 5 g ACN. The solids were dried in a vacuum oven at 40° C. overnight. 5.52 g off-white solids were obtained. The solids were analyzed by XRPD and found to contain tenofovir alafenamide monofumarate, GS-7339 monofumarate, and tenofovir alafenamide hemifumarate.

### Synthetic Example 7

### Preparation of Tenofovir Alafenamide Hemifumarate Via Selective Crystallization

9-[(R)-2-[[[[(S)-1-(isopropoxycarbonyl)ethyl]amino]phenoxyphosphinyl]methoxy]propyl]adenine as a slurry in ACN (9.7 kg slurry, 13.8 wt %, a diastereomeric mixture of 1.0 kg (2.10 mol, 1 mol equiv) of 9-[(R)-2-[[(S)-[[(S)-1-(isopropoxycarbonyl)ethyl]amino]phenoxyphosphinyl]methoxy]propyl]adenine and 0.35 kg of 9-[(R)-2-[[(R)-[[(S)-1-(isopropoxycarbonyl)ethyl]amino]phenoxyphosphinyl]methoxy]propyl]adenine was charged into a reactor and rinsed forward with dichloromethane (5 kg). The mixture was concentrated under vacuum to about 3 L with jacket temperature below 40° C. The concentrate was then coevaporated with ACN (6 kg) under vacuum to about 3 L with jacket temperature below 40° C. The concentrate was diluted with ACN (8.5 kg) and warmed to 40-46° C. The warm mixture was filtered into a second reactor and the filtrate was cooled to 19-25° C.

To the above solution was charged fumaric acid (0.13 kg, 1.12 mol, 0.542 mole equiv) followed by ACN (1 kg), and the mixture was heated to 67-73° C. The hot mixture was transferred into a reactor via a polishing filter, and then adjusted to 54-60° C. Seed crystals (5 g) of the hemifumarate form of tenofovir alafenamide were charged (for example, the mixture can be seeded with tenofovir alafenamide hemifumarate formed in Synthetic Example 6 or a subsequent production), and the resulting mixture was agitated at 54-60° C. for about 30 minutes. The mixture was cooled over a minimum of 4 hours to 0-6° C., and then agitated at 0-6° C. for a minimum of 1 hour. The resulting slurry was filtered and rinsed with chilled (0-6° C.) ACN (2 kg). The product was dried under vacuum below 45° C. until loss on drying (LOD) and organic volatile impurities (OVI) limits were met (LOD ≦1.0%, dichloromethane content ≦0.19%, acetonitrile content ≦0.19%) to afford the final compound of the hemifumarate form of tenofovir alafenamide as a white to off-white powder (typical yield is about 0.95 kg). ¹H NMR (400 MHz, d6 DMSO): δ 1.06 (d, J=5.6 Hz, 3H), 1.12-1.16 (m, 9H), 3.77 (dd, J=10.4, 11.6 Hz, 1H), 3.84-3.90 (m, 2H), 3.94 (m, 1H), 4.14 (dd, J=6.8, 14.8 Hz, 1H), 4.27 (m, 1H), 4.85 (heptet, J=6.0 Hz, 1H), 5.65 (t, J=11.2 Hz, 1H), 6.63 (s, 1H), 7.05 (d. J=7.6 Hz, 2H), 7.13 (t, J=7.2 Hz, 1H), 7.24 (s, 2H), 7.29 (t, J=7.6 Hz, 2H), 8.13 (t, J=13.6 Hz, 2H), ³¹P NMR (162 MHz, d6 DMSO): δ 23.3.

### Synthetic Example 8

### Preparation of Tenofovir Alafenamide Hemifumarate

To a jacketed reactor equipped with overhead agitator, was charged 9-[(R)-2-[[(S)-[[(S)-1-(isopropoxycarbonyl)ethyl]amino]phenoxyphosphinyl]methoxy]propyl]adenine (10 g), fumaric acid (1.22 g), and ACN (100 mL). The mixture was heated to 70-75° C. to dissolve the solids. Any undissolved particulates were removed by filtration through a cartridge filter. The filtered solution was cooled to 60-65° C., and seeded with 1% (by weight) of tenofovir alafenamide hemifumarate. The slurry was aged for 30 minutes and cooled to 0-5° C. over 2 hours. The temperature was maintained for 1-18 hours, and the resulting slurry was filtered and washed with 2 ml of cold ACN (0-5° C.). The solids were dried under vacuum at 50° C. to provide the hemifumarate form of tenofovir alafenamide, which was characterized as described below.

Characterization of Tenofovir Alafenamide Hemifumarate from Synthetic Example 8

Tenofovir alafenamide hemifumarate from Synthetic Example 8 consists of 9-[(R)-2-[[(S)-[[(S)-1-(isopropoxycarbonyl)ethyl]amino]phenoxyphosphinyl]methoxy]propyl]adenine and one-half an equivalent of fumaric acid. Tenofovir alafenamide hemifumarate is anhydrous, nonhygroscopic, and has a DSC onset endotherm of about 131° C.

### X-ray Powder Diffraction

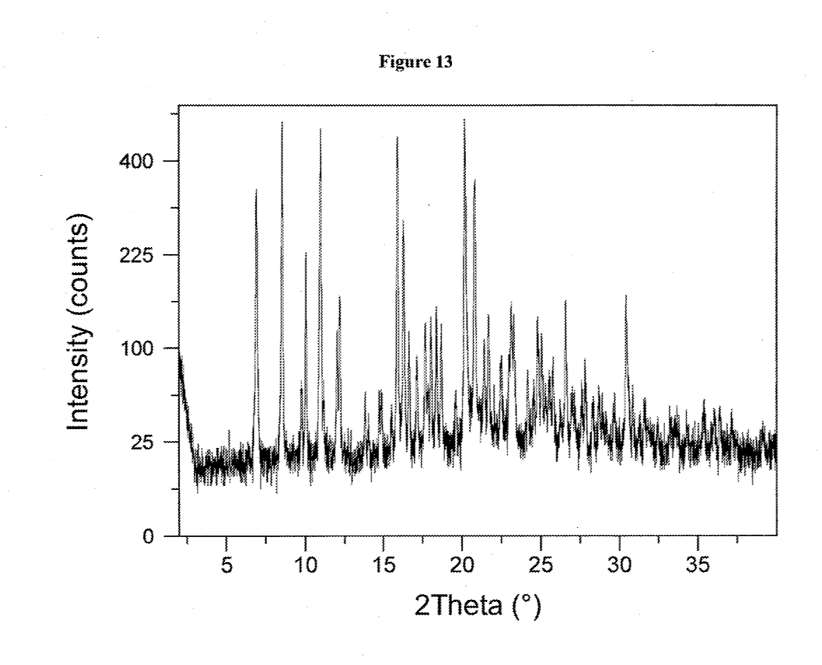

The XRPD pattern of tenofovir alafenamide hemifumarate was obtained in the following experimental setting: 45 KV, 45 mA, Kα1=1.5406 Å, scan range 2-40°, step size 0.0084°, counting time: 8.25 s. The XRPD pattern for tenofovir alafenamide hemifumarate is shown in FIG. 13. The characteristic peaks include: 6.9±0.2°, 8.6±0.2°, 10.0±0.2°, 11.0±0.2°, 12.2±0.2°, 15.9±0.2°, 16.3±0.2°, 20.2±0.2°, and 20.8±0.2°.

### Single-Crystal X-Ray Diffraction

The crystal size was 0.32×0.30×0.20 mm³. The sample was held at 123 K and the data was collected using a radiation source with a wavelength of 0.71073 Å in the theta range of 1.59 to 25.39°. Conditions of and data collected from the single-crystal X-ray diffraction are shown in Table 1.

TABLE 1
Single-Crystal X-ray Diffraction

| | | |
|---|---|---|
| Empirical formula | C₂₃H₃₁N₆O₇P | |
| Formula weight | 534.50 | |
| Temperature | 123(2) K | |
| Crystal size | 0.32 × 0.30 × 0.20 mm³ | |
| Theta range for data collection | 1.59 to 25.39° | |
| Wavelength | 0.71073 Å | |
| Crystal system | Tetragonal | |
| Space group | P4(2)2(1)2 | |
| Unit cell dimensions | a = 18.1185(12) Å | α = 90° |
| | b = 18.1185(12) Å | β = 90° |
| | c = 17.5747(11) Å | γ = 90° |
| Volume | 5769.4(6) Å³ | |
| Z | 8 | |
| Density (calculated) | 1.231 g/cm³ | |

### DSC Analysis

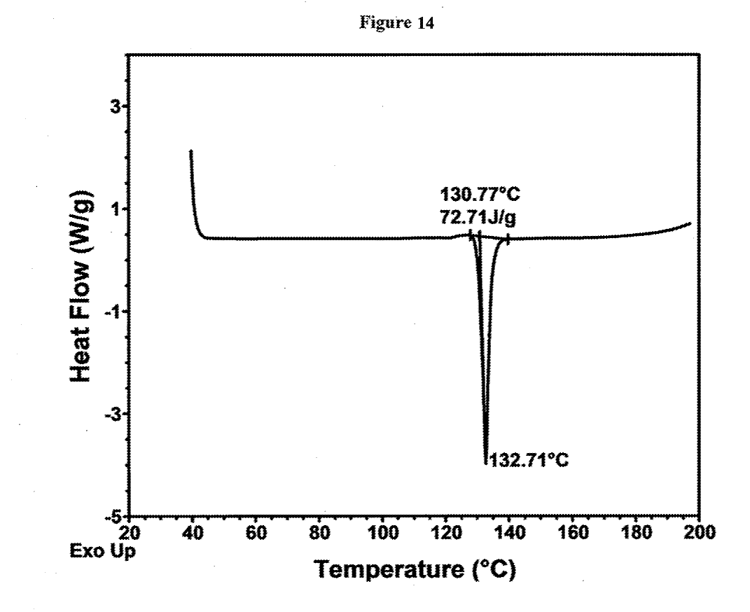

The DSC analysis was conducted using 2.517 mg of tenofovir alafenamide hemifumarate. It was heated at 10° C./min over the range of 40-200° C. The onset endotherm was found to be about 131° C. (FIG. 14).

### TGA Data

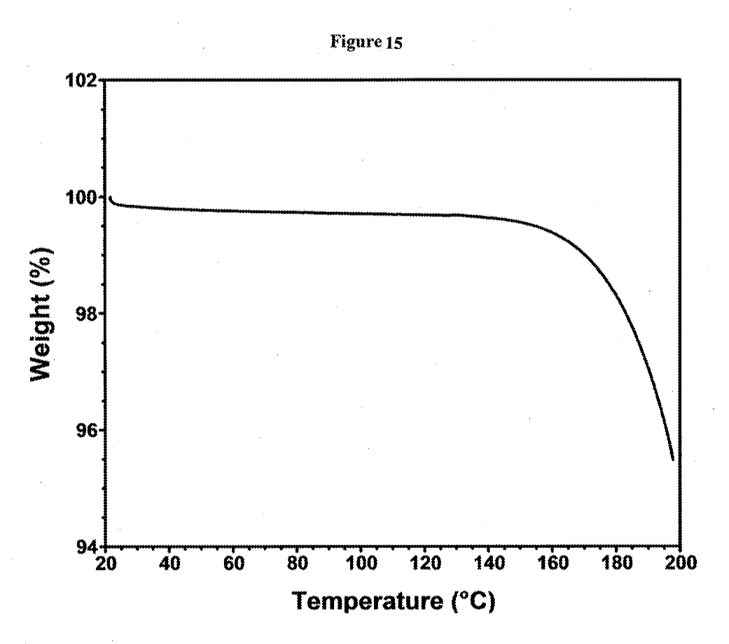

The TGA data were obtained using 4.161 mg of tenofovir alafenamide hemifumarate. It was heated at 10° C./min over the range of 25-200° C. The sample lost 0.3% weight before melting (FIG. 15). It was determined to be an anhydrous form.

### DVS Analysis

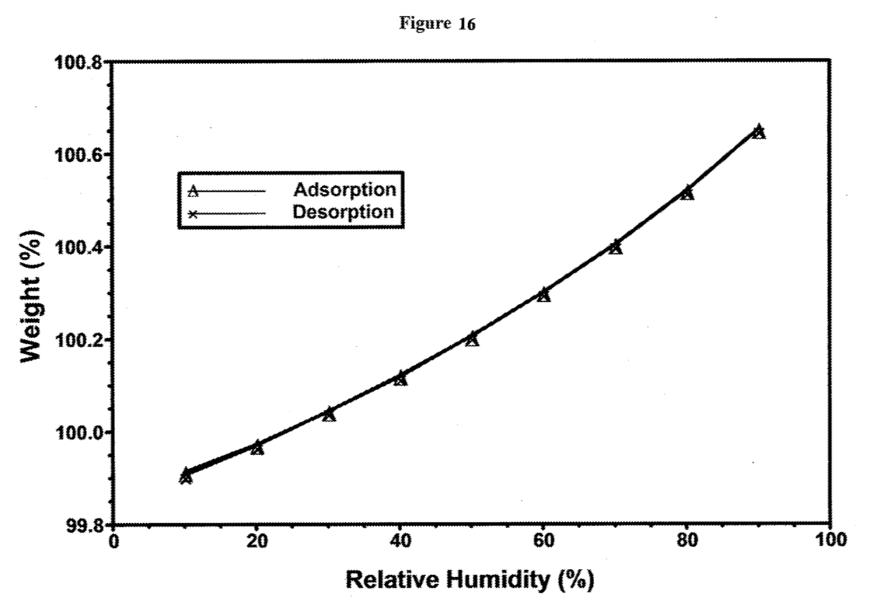

DVS analysis was conducted using 4.951 mg of tenofovir alafenamide hemifumarate. The material was kept at 25° C. in nitrogen at humidities ranging from 10% to 90% relative humidity; each step was equilibrated for 120 minutes. The sorption isotherm is shown at FIG. 16. The material was found to be nonhygroscopic, and to absorb 0.65% water at a relative humidity of 90%.

### Purging of Diastereomeric Impurity

In the prior syntheses of tenofovir alafenamide, one of the major impurities is typically the diastereomer 9-[(R)-2-[[(R)-[[(S)-1-(isopropoxycarbonyl)ethyl]amino]phenoxyphosphinyl]methoxy]propyl]adenine. The hemifumarate form of tenofovir alafenamide from Synthetic Example 8 has an exceptional capability to purge this diastereomeric impurity, as compared with the capability of the monofumarate form (described in, e.g., U.S. Pat. No. 7,390,791). The data in Table 2 (below) demonstrates that tenofovir alafenamide hemifumarate (Batch 2) purged the diastereomeric impurity to less than one-tenth of the starting concentration, whereas the monofumarate form of tenofovir alafenamide (Batch 1) only slightly purged the diastereomeric impurity.

TABLE 2
Purging Capability Comparison

| | Diastereo- | | Fumaric | | |
|---|---|---|---|---|---|
| | meric | | acid | | Diastereo- |
| | Impurity in | | charge | | meric |
| | Starting | | (mole | Product | Impurity in |
| Batch | Material | Solvent | equivalent) | obtained | Product |
| 1 | 9.3% | ACN | 0.9 | Monofumarate | 7.6% |
| | | | | form | |
| 2 | 10.0% | ACN | 0.5 | Hemifumarate | 0.65% |
| | | | | form | |

### Chemical Stability

Chemical stability of the hemifumarate form of tenofovir alafenamide was compared with the monofumarate form. As shown in Table 3 (below), under identical conditions, the hemifumarate form of tenofovir alafenamide was chemically more stable and exhibited better long-term storage stability, with significantly less degradation (% Total Deg. Products) than the monofumarate form. Conditions evaluated include temperature, relative humidity (RH), and the open or closed state of the container cap.

TABLE 3
Chemical Stability Comparison

| | | Monofumarate form | | Hemifumarate form | |
|---|---|---|---|---|---|
| | Time | % TA* | % Total | % TA | % Total |
| Storage | Points | Area | Deg. | Area | Deg. |
| Condition | (weeks) | Normalized | Products | Normalized | Products |
| 40° C./75% | 0 | 97.1 | 0.69 | 98.4 | 0.05 |
| RH Cap Closed | 1 | 97.0 | 0.87 | 98.4 | 0.14 |
| | 2 | 96.6 | 1.18 | 98.5 | 0.14 |
| | 4 | 96.4 | 1.49 | 98.4 | 0.25 |
| | 8 | 95.4 | 2.36 | 98.0 | 0.49 |
| 40° C./75% | 0 | 97.1 | 0.69 | 98.4 | 0.05 |
| RH Cap Open | 1 | 96.9 | 0.90 | 98.5 | 0.15 |
| | 2 | 96.6 | 1.10 | 98.5 | 0.14 |
| | 4 | 96.2 | 1.67 | 98.4 | 0.26 |
| | 8 | 95.0 | 2.74 | 98.1 | 0.50 |
| 70° C. | 0 | 97.1 | 0.69 | 98.4 | 0.05 |
| Cap Closed | 2 | 96.2 | 1.83 | 98.5 | 0.22 |
| | 4 | 93.3 | 4.78 | 98.4 | 0.33 |

*TA is tenofovir alafenamide

### Thermodynamic Stability

Stable form screening of tenofovir alafenamide hemifumarate showed that it is thermodynamically stable in most solvents, such as ACN, toluene, ethyl acetate, methyl tert-butyl ether (MTBE), acetone, THF, and 2-methyl THF. A similar stable form screening of the monofumarate form showed that this form is not thermodynamically stable in the above-listed solvents. When suspended in these solvents, the monofumarate form of tenofovir alafenamide fully converts to the hemifumarate form in THF and 2-methyl THF, and partially converts to the hemifumarate form in ACN, ethyl acetate, MTBE, and acetone, as well as at ambient temperatures.

### Thermal Stability

As shown by the DSC data, the hemifumarate form of tenofovir alafenamide has a melting point that is about 10° C. higher than that of the monofumarate form, indicating that the hemifumarate form has improved thermal stability as compared with the monofumarate form.

### Biological Example 1

### Transport Studies

Caco-2 transepithelial transport studies: Caco-2 cells between passage 43 and 69 were grown to confluence over at least 21 days on 24-well polyethylene-terephthalate (PET) transwell plates (BD Biosciences, Bedford, Mass.). Experiments were conducted using Hank's Buffered Salt Solution (HBSS) containing 10 mM HEPES and 15 mM Glucose obtained from Life Technologies (Grand Island, N.Y.). Donor and receiver buffers had their pH adjusted to pH 6.5 and 7.4, respectively. The receiver well used HBSS buffer supplemented with 1% bovine serum albumin. In studies done to determine transport inhibition, monolayers were preincubated for 60 minutes in the presence of assay buffer and inhibitor in order to saturate any transporter binding sites. Following preincubation, fresh assay buffer containing inhibitor and the test compound were added. Test compound concentrations in assay chambers were analyzed by liquid chromatography coupled to tandem mass spectrometry (LC/MS/MS). Transepithelial electrical resistance (TEER) and lucifer yellow permeability were determined to assure membrane integrity. Each individual experiment was done in duplicate and the permeation of control compounds atenolol (low permeability), propranolol (high permeability), and vinblastine (efflux transport) were determined to meet acceptance criteria for each batch of assay plates.

Pgp and BCRP inhibition assays in transfected Madin-Darby canine kidney (MDCKII) cells: Inhibition of Pgp-mediated transport was studied using the Pgp substrate calcein AM and MDCKII cells transfected with the human MDR1 (ABCB1) gene (encoding Pgp). Similarly, inhibition of BCRP-mediated transport was studied using the BCRP substrate Hoechst 33342 and MDCKII cells transfected with the human ABCG2 gene (encoding BCRP). Briefly, MDCKII cells were seeded in 96-well black cell culture plates with clear bottoms at a density of 5×10⁴ cells/well and grown to confluence overnight. Test compounds were diluted in cell culture medium containing 10 μM Hoechst 33342 and incubated for 3 hours with MDCKII-BCRP and nontransfected cells. Following removal of media containing Hoechst 33342 and test compound, cells were washed twice with warm medium and lysed at room temperature for 5-10 minutes in a buffer containing 20 mM Tris-HCl pH 9.0 and 0.4% Triton X-100. Wells were analyzed for Hoechst 33342 fluorescence at an excitation of 353 nm and an emission of 460 nm.

Pgp and BCRP substrate assays in transfected MDCKII cells: MDCKII cells were grown to confluence over 4-6 days on 24-well PET transwell plates (BD Biosciences). The same buffers were used in the donor and receiver wells as described above for caco-2 studies. Experiments were conducted as described above for caco-2 transepithelial transport studies and samples analyzed by LC/MS/MS. Similar quality control and acceptance criteria were used as those described above for caco-2 studies. TEER values and the permeability of lucifer yellow, atenolol, and propranolol were determined to meet acceptance criteria for each batch of assay plates. Efflux ratios were determined to be at least 3-fold higher in transfected versus nontransfected monolayers for the model Pgp substrate vinblastine and BCRP substrate prazosin.

Data analysis: The 50% inhibition constants (IC₅₀) values for transporters in the fluorescent accumulation studies done in MDCKII cells, defined as the test article concentration needed to inhibit the maximal transporter specific transport by 50%, were calculated using nonlinear curve fitting of inhibition versus concentration to a sigmoidal curve with a variable Hill coefficient using GraphPad Prism 5 (GraphPad Software Inc., San Diego, Calif.). Apparent permeability coefficients and efflux ratios (ER) from transcellular experiments in caco-2 or MDCKII cells were calculated as previously described (Tong et al. (2007) Antimicrob Agents Chemother 51:3498-504). Where appropriate, the statistical significance of differences observed between test conditions was assessed using paired two-tailed Student's t tests.

Inhibition of Pgp and BCRP in transfected MDCKII cells: The inhibition of Pgp and BCRP by cobicistat relative to ritonavir and the known transport inhibitors cyclosporin A (CSA) and fumitremorgin C was studied by monitoring the effects of coincubation on the Pgp- and BCRP-dependent accumulation of the fluorescent probe substrates calcein AM and Hoechst 33342 in MDCKII-MDR1 and MDCKII-ABCG2 cells, respectively. Cobicistat inhibited Pgp and BCRP with IC₅₀ values of 36±10 μM and 59±28 μM, respectively. Ritonavir, when incubated at its approximate solubility limit in assay buffers (20 μM) showed 35% inhibition of Pgp and 21% inhibition of BCRP. Higher concentrations of cobicistat were achievable in assays because of its >35-fold higher aqueous solubility at neutral pH. Greater differences in the concentrations of cobicistat and ritonavir may exist in the gastrointestinal (GI) tract based on their respective solubility under acidic conditions. Taken together, the solubility and inhibition results indicate that cobicistat should have similar inhibition of Pgp and BCRP in the GI tract relative to ritonavir.

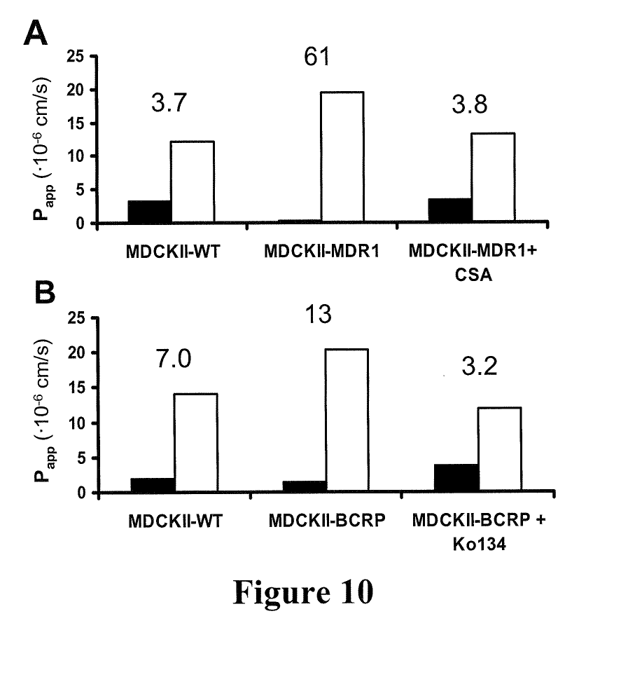

Pgp and BCRP substrate assays in transfected MDCKII cells: To further characterize the mechanism interaction of cobicistat with Pgp (multidrug resistance protein 1; MDR1) and BCRP, bidirectional permeability assays were completed in cells transfected with the genes for the human transport proteins to determine if cobicistat is a substrate for these efflux transporters (FIG. 10). Bidirectional permeability of cobicistat (10 μM) was assessed in MDCKII-WT, MDCKII-MDR1 (FIG. 10A) and MDCKII-BCRP cells (FIG. 10B). The black bars show apical to basolateral (A-B) permeability, and the open bars show basolateral to apical (B-A) permeability. Efflux ratios are indicated above graphs for each experimental condition. CSA (10 μM) and Ko134 (10 μM) were used as known inhibitors of Pgp and BCRP, respectively. Results are the average of duplicate wells from a representative side by side experiment done comparing wild type MDCKII (MDCKII-WT) to MDCKII-MDR1 or MDCKII-BCRP cells in the presence or absence of respective inhibitors. The overexpression of Pgp or BCRP in MDCKII cells increased the efflux ratios of cobicistat. These increased efflux ratios reflected a decrease in the forward permeability and an increase in the reverse permeability of cobicistat. Consistent with Pgp- and BCRP-dependent transport, cobicistat efflux was decreased in the presence of the Pgp inhibitor CSA and the BCRP inhibitor Ko 134. These results illustrate that cobicistat is a substrate for both Pgp and BCRP, suggesting that the observed inhibition may be due to competition for the binding sites of the respective transporters.

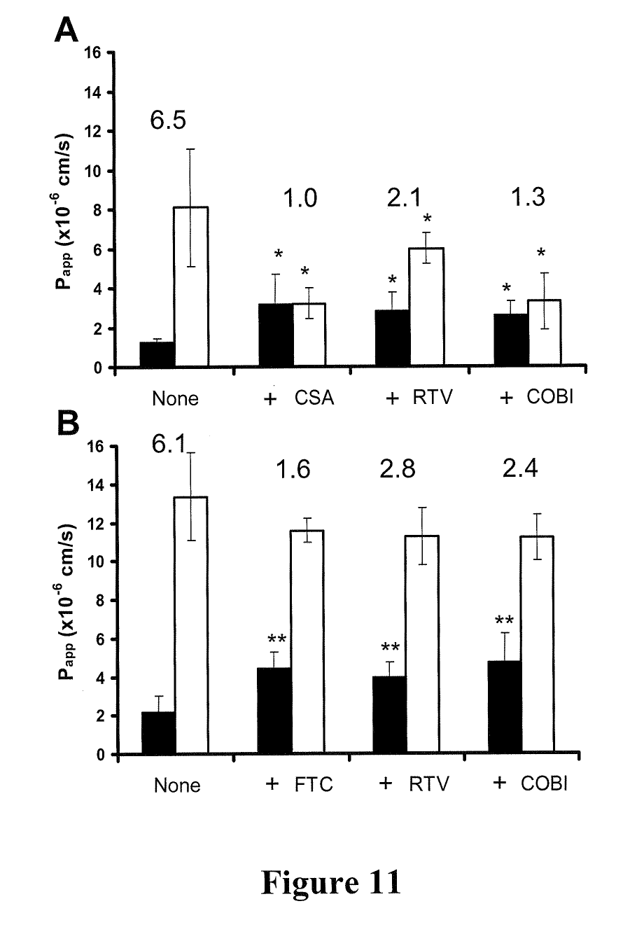

Effect of cobicistat on the bidirectional permeability of model Pgp and BCRP substrates through caco-2 cell monolayers: Caco-2 cells have been reported as a physiologically relevant model system of GI absorption that supports the polarized expression of intestinal transporters including Pgp and BCRP. The effect of cobicistat (COBI; 90 μM) and ritonavir (RTV; 20 μM) on the bidirectional permeability through monolayers of caco-2 cells of 10 μM of the Pgp substrate digoxin (FIG. 11A) and BCRP substrate prazosin (FIG. 11B) were studied. Digoxin and prazosin were chosen as model substrates of Pgp and BCRP, respectively, based on recommendations from the FDA and by the International Transporter Consortium. The known Pgp inhibitor CSA (10 μM) and BCRP inhibitor fumitremorgin C (2 μM; noted in FIG. 11B as “FTC”) were used as positive controls. The black bars show apical to basolateral (A-B) and the open bars basolateral to apical (B-A) permeability, and efflux ratios are indicated above graphs for each experimental condition. Results are the mean±standard deviation of at least four independent experiments done in duplicate, and statistical significance was assessed by comparing results to no cotreatment wells using paired two-tailed Student's t tests (*, P<0.05; **, P<0.01). Similar to the known Pgp inhibitor CSA, cobicistat and ritonavir markedly reduced the efflux ratio and significantly increased the apical to basolateral (A-B) permeability of digoxin (FIG. 11A). Similar effects were observed in experiments studying the effect of cobicistat and ritonavir relative to the known BCRP inhibitor fumitremorgin C on the permeability of the BCRP substrate prazosin (FIG. 11B). These data suggest similar inhibitory effects of cobicistat and ritonavir on the Pgp-mediated transport of digoxin- and BCRP-mediated transport of prazosin.

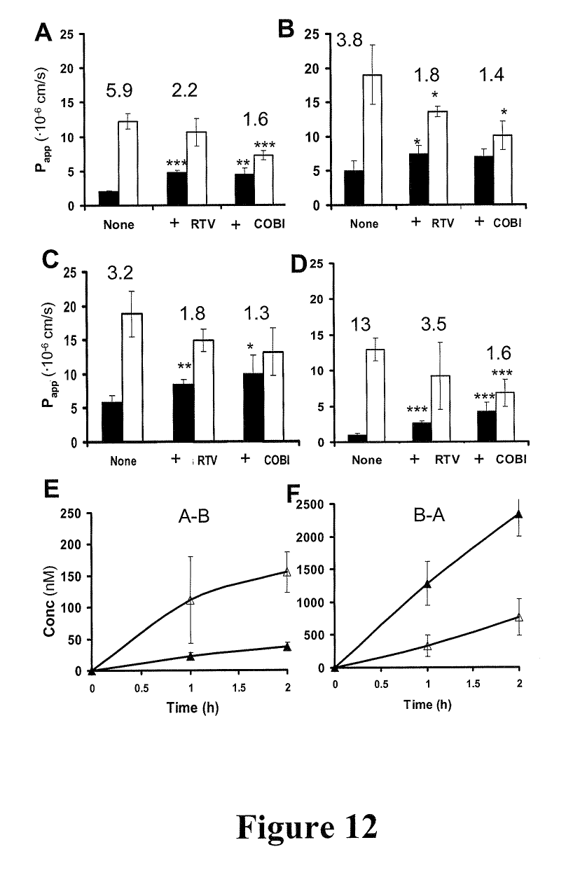

Effect of cobicistat on the bidirectional permeability of HIV protease inhibitors and GS-7340 through caco-2 cell monolayers: The effect of cobicistat (90 μM) and ritonavir (20 μM) on the bidirectional permeability of the HIV protease inhibitors (PIs) atazanavir, darunavir, lopinavir, and GS-8374, an experimental HIV PI, through caco-2 cell monolayers was assessed. The effect of RTV and COBI was assessed with 10 μM of the HIV PIs atazanavir (FIG. 12A), darunavir (FIG. 12B), lopinavir (FIG. 12C) and GS-8374 (FIG. 12D). The black bars show apical to basolateral (A-B) and the open bars basolateral to apical (B-A) permeability, and efflux ratios are indicated above graphs for each experimental condition. Results are the mean±standard deviation of at least four independent experiments done in duplicate, and statistical significance was assessed comparing directional results to no cotreatment wells by using paired two-tailed Student's t tests (*, P<0.05; **, P<0.01; ***, P<0.001). The effect of COBI (90 μM) was assessed on the bidirection permeability of GS-7340 (10 μM) through caco-2 monolayers over a 2 hour time course in the A-B (FIG. 12E) and B-A (FIG. 12F) directions. Open symbols depict presence and solid symbols depict absence of COBI. Results are the mean±standard deviation of duplicate measurements from two independent experiments. Consistent with previous studies reporting these compounds as Pgp substrates, significant efflux was observed for each of the protease inhibitors. Coadministration of cobicistat and ritonavir comparably reduced the efflux ratios by increasing the A-B flux and decreasing the B-A flux of the protease inhibitors (FIG. 12A-D). The effect of cobicistat on GS-7340 permeability across caco-2 monolayers was monitored over 2 hours, and cobicistat increased the A-B flux of GS-7340 while concomitantly reducing B-A flux (FIG. 12E-F).

These results support the hypothesis that cobicistat may be acting to inhibit Pgp-mediated intestinal secretion of GS-7340.

### Biological Example 2

Pharmacokinetic studies were done in humans to determine exposure to GS-7340 at three dose levels. Eligible subjects were randomized to receive either GS-7340 dose of 8 mg, GS-7340 dose of 25 mg, GS-7340 dose of 40 mg, tenofovir (as TDF) 300 mg or placebo-to-match GS-7340 for 10 days. (Note: Doses of GS-7340 are given as the mass of free base of GS-7340, even where other forms of GS-7340 were dosed.) GS-7340 was administered in a blinded fashion, unless a subject was randomized to receive tenofovir which was given on an open-label basis.

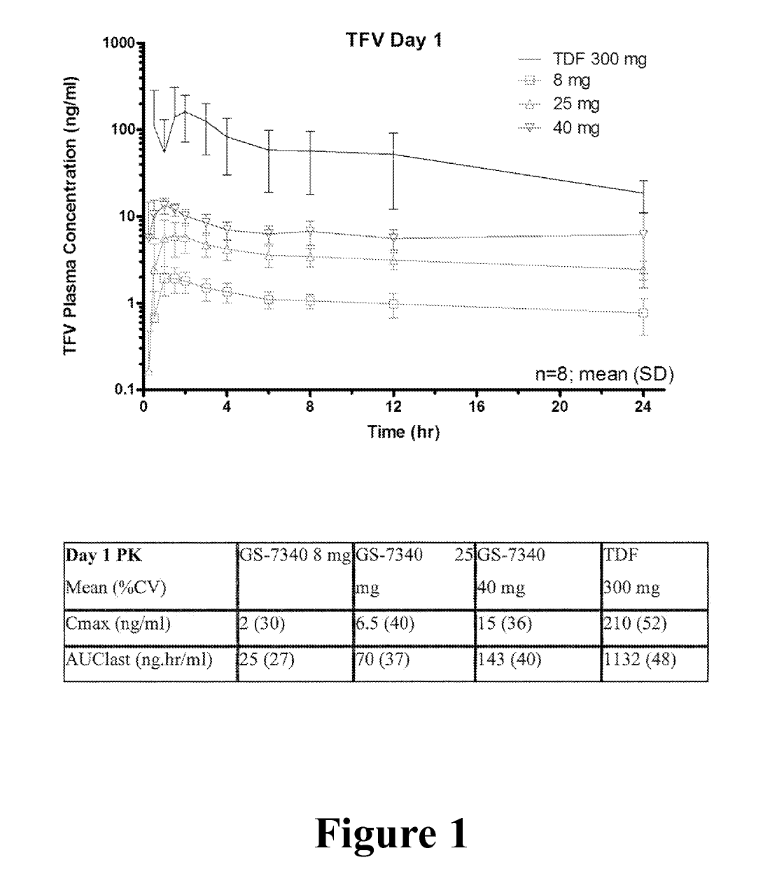

FIG. 1 shows tenofovir plasma concentrations in patients on Day 1 of the study. The top line (no symbol) shows the concentration of tenofovir in patients dosed with 300 mg tenofovir (as TDF). The next line down (triangles pointed down) shows the concentration of tenofovir in patients dosed with 40 mg GS-7340. The next line down (triangles pointed up) shows the concentration of tenofovir in patients dosed with 25 mg GS-7340. The bottom line (squares) shows the concentration of tenofovir in patients dosed with 8 mg GS-7340. The table below the graph shows Cmax and AUC values obtained.

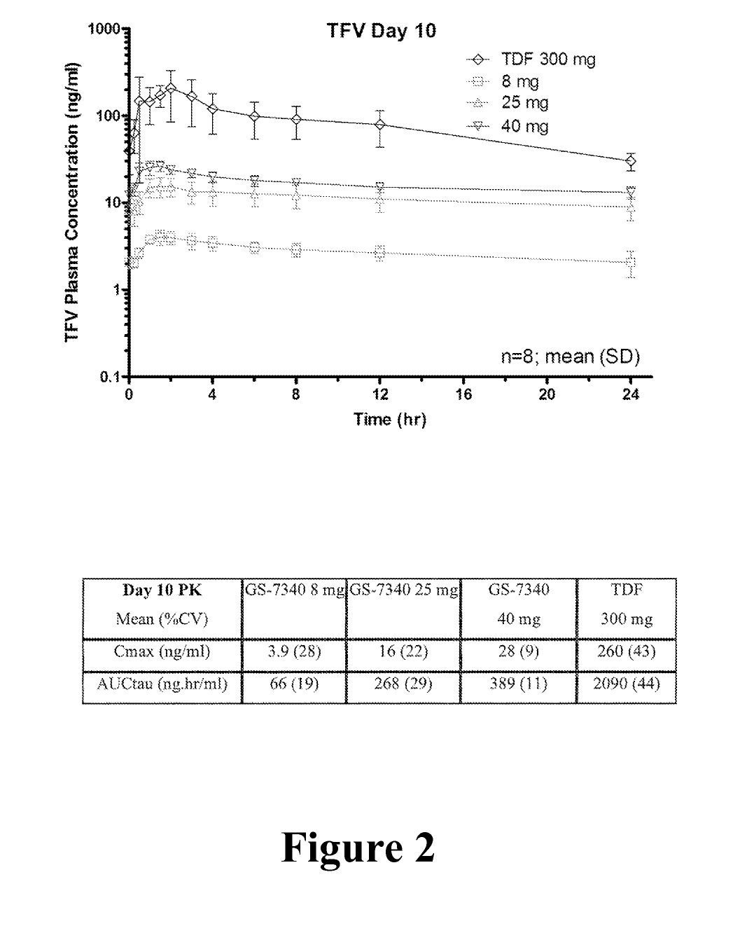

FIG. 2 shows tenofovir plasma concentrations in patients on Day 10 of the study. The top line (diamonds) shows the concentration of tenofovir in patients dosed with 300 mg tenofovir. The next line down (triangles pointed down) shows the concentration of tenofovir in patients dosed with 40 mg GS-7340. The next line down (triangles pointed up) shows the concentration of tenofovir in patients dosed with 25 mg GS-7340. The bottom line (squares) shows the concentration of tenofovir in patients dosed with 8 mg GS-7340. The table below the graph shows Cmax and AUC values obtained.

### Biological Example 3

Drug interaction potential between once-daily emtricitabine (FTC)/GS-7340 fixed dose combination, cobicistat boosted darunavir plus GS-7340 as a single agent, and efavirenz or cobicistat-boosted darunavir was evaluated in an open-label, crossover, single-center, multiple-dose, multiple-cohort study.

Table 4 shows the dosing regimen and schedule for the study.

TABLE 4
Cohort 1 (n = 12)

| Cohort | Day 1-12 | Day 13-26 |
|---|---|---|
| | Treatment A: FTC/GS-7340 FDC (200/40 | Treatment B: FTC/GS-7340 FDC (200/40 mg) plus |
| | mg) administered once-daily in the | efavirenz (EFV) 600 mg administered once-daily in |
| | morning under fasted condition | the morning under fasted condition |

Cohort 2 (n = 12)

| Cohort | Day 1-12 | Day 13-22 |
|---|---|---|
| | Treatment C: FTC/GS-7340 FDC (200/25 | Treatment D: FTC/GS-7340 FDC (200/25 mg) plus |
| | mg) administered once-daily in the | cobicistat-boosted darunavir (DRV/co; 800/150 mg) |
| | morning under fed condition | administered once-daily in the morning under fed |
| | | condition |

Cohort 3 (n = 14)

| Cohort | Day 1-10 | Day 11-22 |
|---|---|---|
| | Treatment E: Cobicistat boosted | Treatment F: FTC/GS-7340 FDC (200/25 mg) plus |
| | darunavir (DRV/co; 800/150 mg) | cobicistat boosted darunavir (DRV/co, 800/150 mg) |
| | administered once-daily in the morning | adminstered once daily in the morning under fed |
| | under fed condtion | condition |

Cohort 4 (n = 12)

| Cohort | Day 1-12 | Day 13-22 |
|---|---|---|
| | Treatment G: GS-7340 (8 mg) single | Treatment H: GS-7340 (8 mg) single agent PLUS |
| | agent administered once daily in the | cobicistat (150 mg) administered once daily in the |
| | morning under fed conditions | morning under fed Conditions |

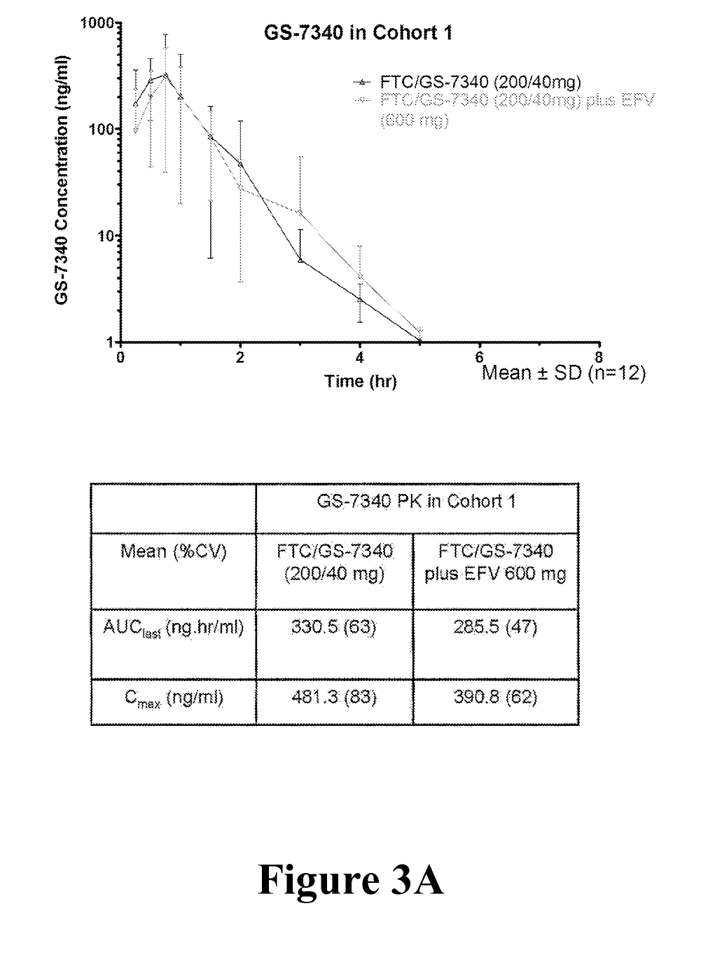

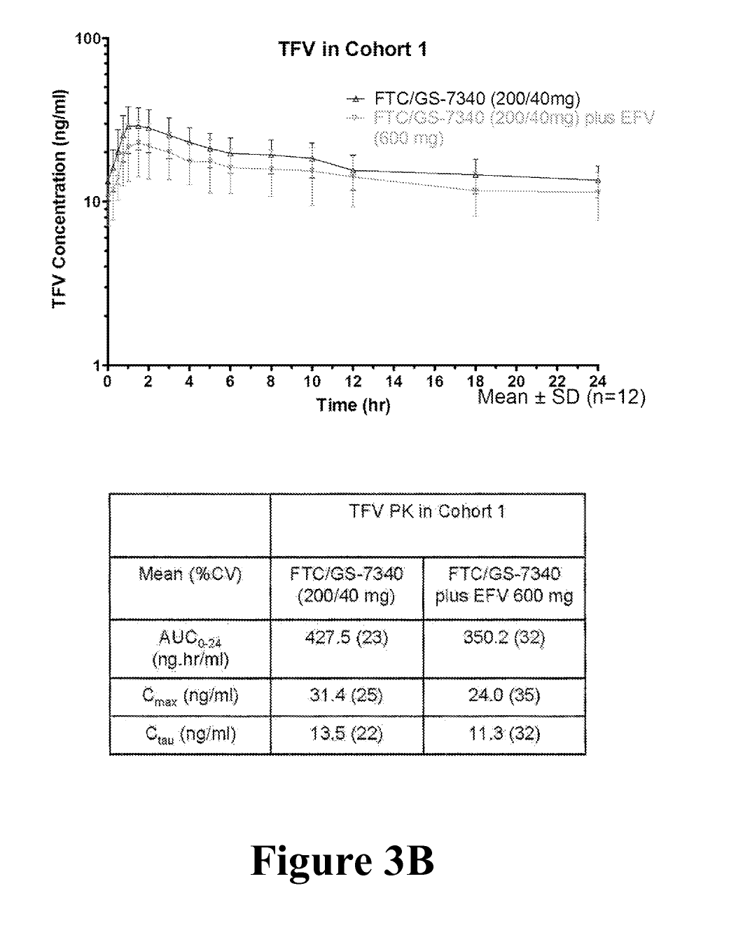

Results of the pharmacokinetic analysis in this study are shown in FIGS. 3-5. (Note: Doses of GS-7340 are given as the mass of free base of GS-7340, even where other forms of GS-7340 were dosed.)

FIG. 3A shows GS-7340 (tenofovir alafenamide) concentrations (ng/ml) for doses of emtricitabine and GS-7340 (triangles pointed up) and emtricitabine, GS-7340 and efavirenz ((initial value=100 ng/ml); triangles pointed down) in patients from Cohort 1. Cₘₐₓ and AUC results are displayed in the table below for GS-7340 exposure. Tenofovir (TFV) concentrations are shown in FIG. 3B for doses of emtricitabine and GS-7340 (upper line; triangles pointed up) and emtricitabine, GS-7340 and efavirenz (lower line: triangles pointed down). Cₘₐₓ and AUC results are displayed in the table below for tenofovir exposure.

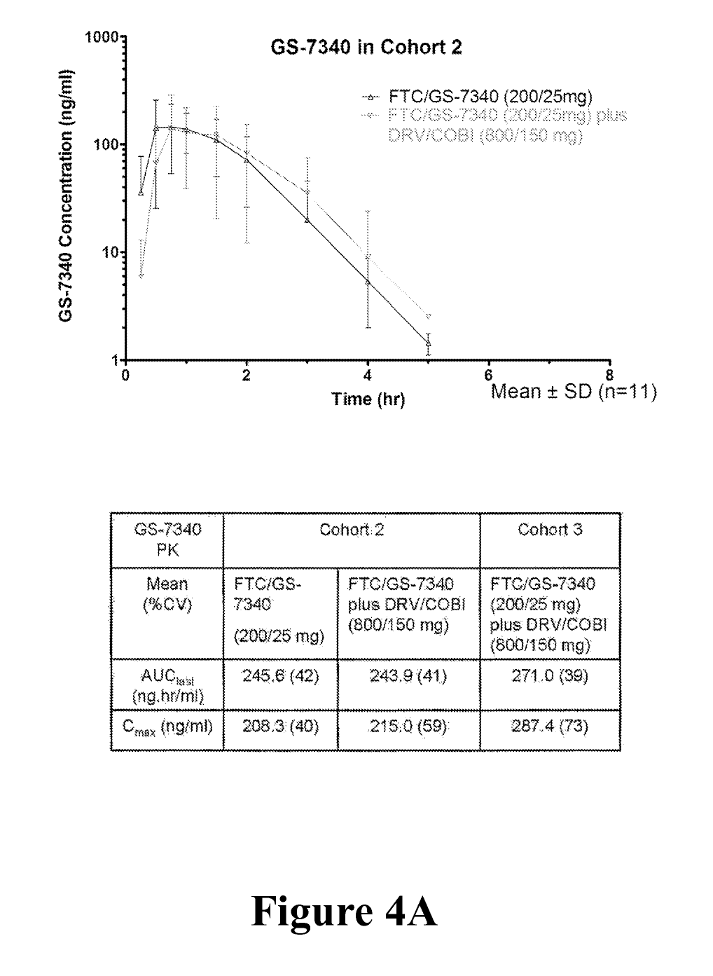

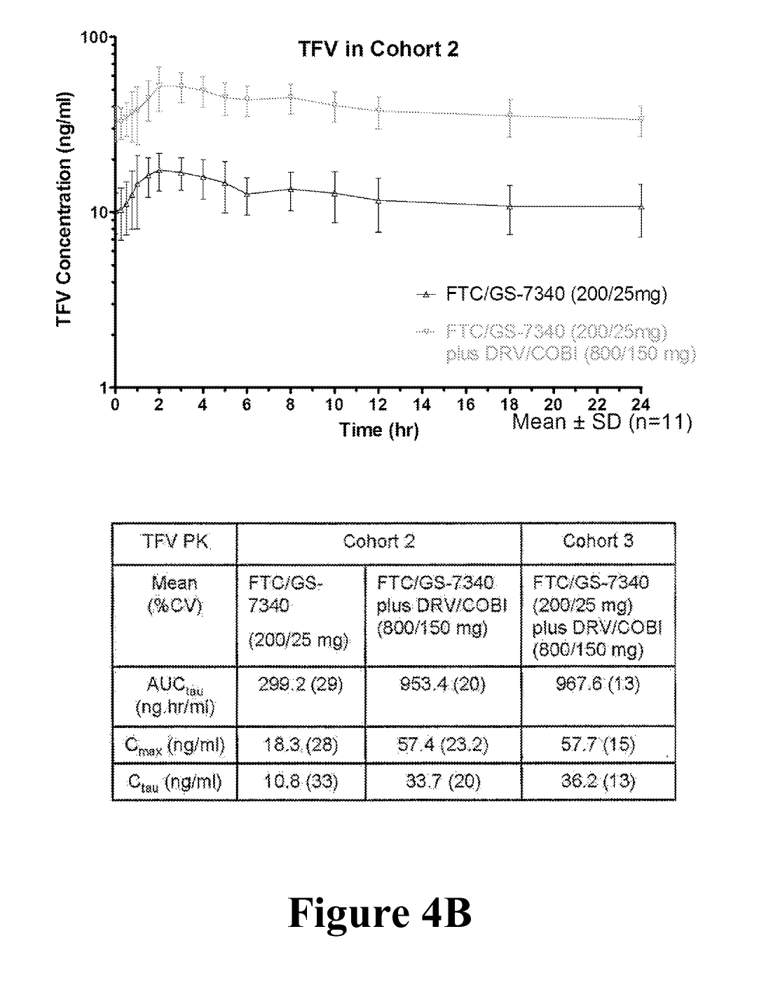

FIG. 4A shows GS-7340 concentrations (ng/ml) for doses of emtricitabine and GS-7340 (triangles pointed up) and emtricitabine, GS-7340, darunavir, and cobicistat (triangles pointed down) in patients from Cohort 2. Cₘₐₓ and AUC results are displayed in the table below for GS-7340 exposure. Tenofovir (TFV) concentrations are shown in FIG. 4B for doses of emtricitabine and GS-7340 (triangles pointed up) and emtricitabine, GS-7340, darunavir, and cobicistat (triangles pointed down). Cₘₐₓ and AUC results are displayed in the table below for tenofovir exposure.

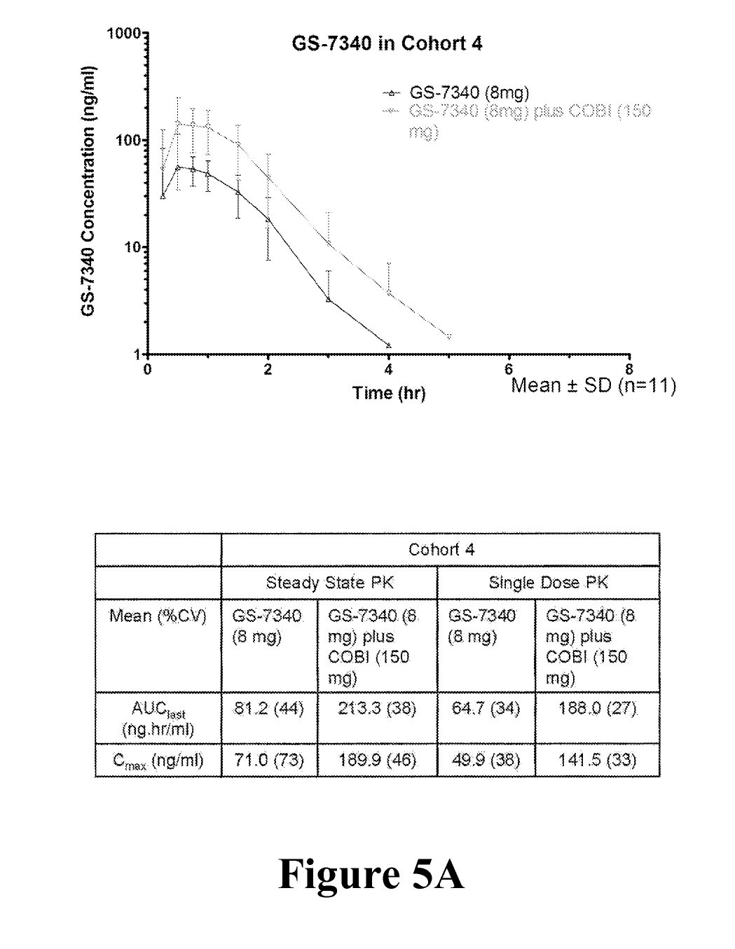

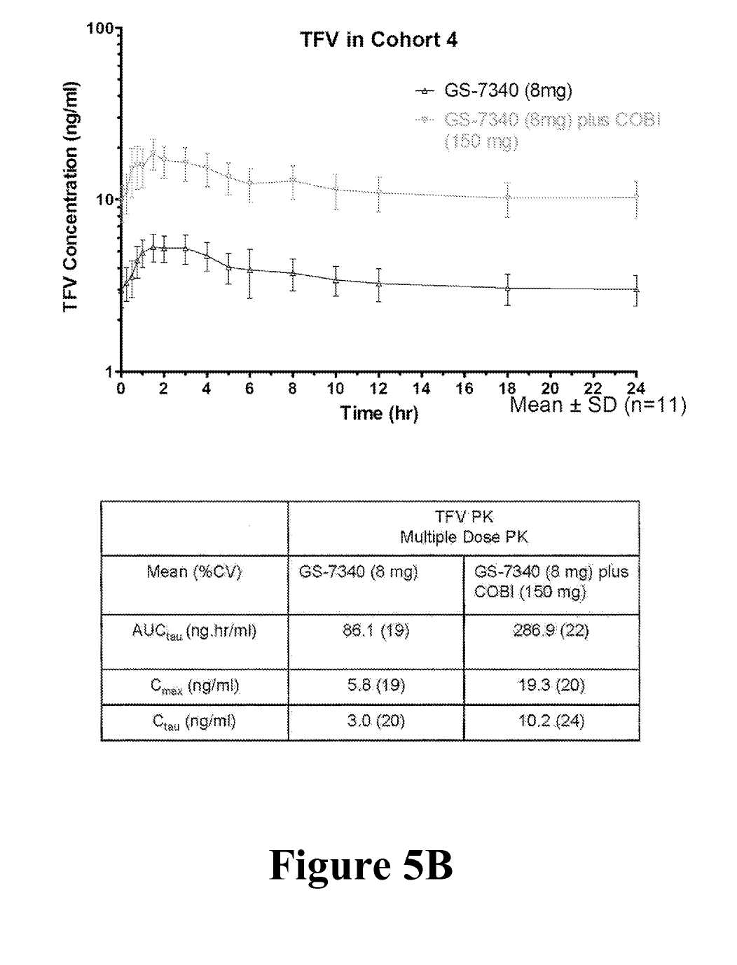

FIG. 5A shows GS-7340 concentrations (ng/ml) for doses of GS-7340 alone and GS-7340 and cobicistat (triangles pointed up). Cₘₐₓ and AUC results are displayed in the table below for GS-7340 exposure. Tenofovir (TFV) concentrations are shown in FIG. 5B for doses of GS-7340 alone (triangles pointed up) and GS-7340 and cobicistat (triangles pointed down). Cₘₐₓ and AUC results are displayed in the table below for tenofovir exposure.

Increases in exposures were observed for GS-7340 (tenofovir alafenamide) and TFV when dosed as GS-7340 (8 mg) plus COBI (150 mg) versus GS-7340 (8 mg) as a stand-alone agent. GS-7340 AUCₗₐₛₜ and Cₘₐₓ were ˜2.7- and 2.8-fold higher, respectively, whereas TFV AUCₜₐᵤ and Cₘₐₓ were ˜3.3- and 3.3-fold higher, respectively. These data suggest that the interaction is COBI-mediated, likely due to inhibition of Pgp-mediated intestinal secretion of tenofovir alafenamide (GS-7340).

### Biological Example 4

GS-7340 and cobicistat were administered in conjunction with elvitegravir and emtricitabine in a clinical trial to determine the relative bioavailability of these compounds. The compounds were administered using a 25 mg or 40 mg dose of GS-7340 (test) relative to exposures (elvitegravir, cobicistat, emtricitabine) from elvitegravir/cobicistat/emtricitabine/tenofovir (reference) or GS-7340 (TFV) (reference). A second cohort with a similar design evaluated an alternate formulation of elvitegravir/cobicistat/emtricitabine/GS-7340 STR. (Note: Doses of Compound are given as the mass of free base of GS-7340, even where other forms of GS-7340 were dosed.) Elvitegravir/cobicistat/emtricitabine/GS-7340 (monolayer) tablets were manufactured by blending of emtricitabine/GS-7340 granulation with elvitegravir granulation and cobicistat, tablet compression, tablet film-coating, and packaging. Elvitegravir/cobicistat/emtricitabine/GS-7340 bilayer tablets are manufactured by compression of the elvitegravir/cobicistat layer and emtricitabine/GS-7340 layer, tablet film-coating, and packaging. In order to provide a robust assessment of pharmacokinetic comparisons between test versus reference treatments, a balanced Williams 4×4 design was used in each cohort.

The dose of elvitegravir (150 mg), the boosting dose of cobicistat (150 mg), and dosage of emtricitabine (200 mg) in elvitegravir/cobicistat/emtricitabine/GS-7340 represent current investigational doses (elvitegravir, cobicistat) or marketed dose (emtricitabine) with demonstrated durable efficacy and long-term safety in HIV-infected patients.

The evaluation used two cohorts of twenty patients. In Cohort 1, the following study treatments were administered.

Treatment A: 1×Single Tablet Regimen (STR) of Formulation 1 (150 mg elvitegravir plus 150 mg cobicistat plus 200 mg emtricitabine plus 25 mg GS-7340 (as 31.1 mg of the fumarate salt GS-7340-02)) QD, administered in A.M. for 12 days.

Treatment B: 1×STR Formulation 1 (150 mg elvitegravir plus 150 mg cobicistat plus 200 mg emtricitabine plus 40 mg GS-7340 (as 49.7 mg of the fumarate salt GS-7340-02)) QD, administered in A.M. for 12 days.

Treatment C: 1×STR (150 mg elvitegravir plus 150 mg cobicistat plus 200 mg emtricitabine plus 300 mg tenofovir (as tenofovir disoproxil fumarate) QD, administered in A.M. for 12 days.

Treatment D: 1×25 mg GS-7340 tablet QD, administered in A.M. for 12 days.

Patients were randomized to one of four sequences (I, II, III, or IV).

| | Day 1-12 | Day 15-26 | Day 29-40 | Day 43-54 |
|---|---|---|---|---|
| Sequence I | A | B | C | D |
| Sequence II | B | D | A | C |
| Sequence III | C | A | D | B |
| Sequence IV | D | C | B | A |

Formulation 1 (monolayer) was prepared by blending of emtricitabine/GS-7340 granulation with elvitegravir granulation and cobicistat, tablet compression, tablet film-coating, and packaging. The EVG/COBI/FTC/GS-7340 STR tablet cores contain colloidal silicon dioxide, croscarmellose sodium, hydroxypropyl cellulose, lactose monohydrate, microcrystalline cellulose, sodium lauryl sulfate, and magnesium stearate as inactive ingredients and are film-coated with polyvinyl alcohol, polyethylene glycol, talc, and titanium dioxide.

In Cohort 2, the following study treatments were administered:

Treatment E: 1×STR Formulation 2 (150 mg elvitegravir plus 150 mg cobicistat plus 200 mg emtricitabine plus 25 mg GS-7340 (as 31.1 mg of the fumarate salt GS-7340-02)) QD, administered in A.M. for 12 days.

Treatment F: 1×STR Formulation 2 (150 mg elvitegravir plus 150 mg cobicistat plus 200 mg emtricitabine plus 40 mg GS-7340 (as 49.7 mg of the fumarate salt GS-7340-02)) QD, administered in A.M. for 12 days.

Treatment C: 1×STR (150 mg elvitegravir plus 150 mg cobicistat plus 200 mg emtricitabine plus 300 mg tenofovir) QD, administered in A.M. for 12 days.

Treatment D: 1×25 mg GS-7340 tablet QD, administered in A.M. for 12 days.

Patients were randomized to one of four sequences (I, II, III, or IV).

| | Day 1-12 | Day 15-26 | Day 29-40 | Day 43-54 |
|---|---|---|---|---|
| Sequence I | E | F | C | D |
| Sequence II | F | D | E | C |
| Sequence III | C | E | D | F |
| Sequence IV | D | C | F | E |

Formulation 2 was prepared as bilayer tablets that were manufactured by compression of the elvitegravir/cobicistat layer and emtricitabine/GS-7340 layer, tablet film-coating, and packaging. The EVG/COBI/FTC/GS-7340 STR tablet cores contain colloidal silicon dioxide, croscarmellose sodium, hydroxypropyl cellulose, lactose monohydrate, microcrystalline cellulose, sodium lauryl sulfate, and magnesium stearate as inactive ingredients and are film-coated with polyvinyl alcohol, polyethylene glycol, talc, and titanium dioxide.

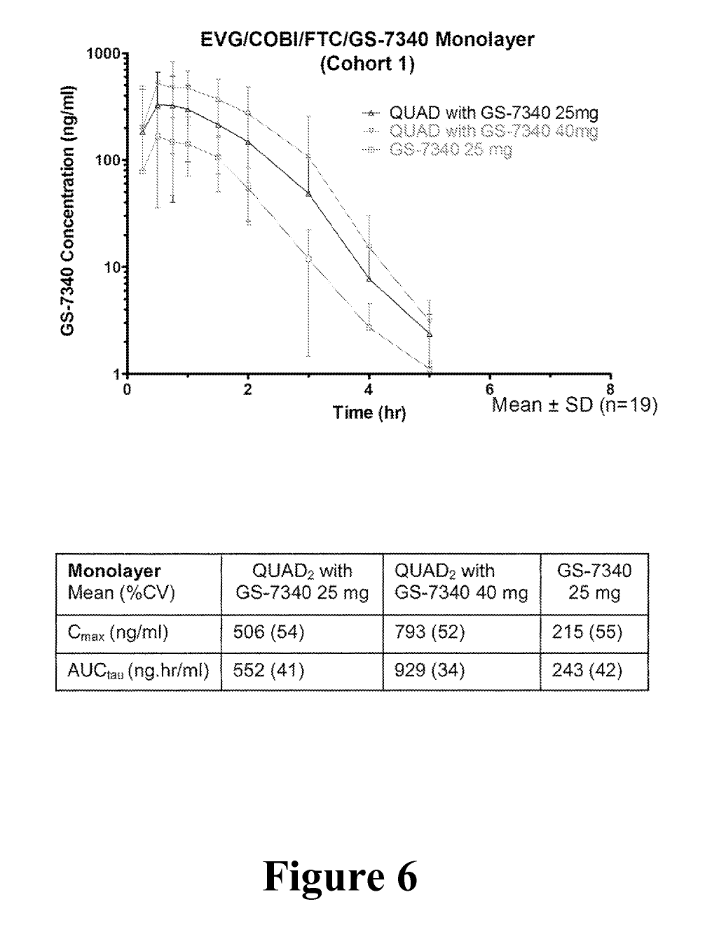

FIG. 6 shows pharmacokinetic data for GS-7340 from patients treated in Cohort 1 (Formulation 1, monolayer). The top line (triangles pointed down) shows GS-7340 concentration (ng/ml) when 40 mg GS-7340 is administered with cobicistat. The middle line (triangles pointed up) shows GS-7340 concentration (ng/ml) when 25 mg GS-7340 is administered with cobicistat. The bottom line (squares) shows GS-7340 concentration (ng/ml) when 25 mg GS-7340 is administered alone. These results show GS-7340 levels that are 2.2-fold higher for dosing at the 25 mg level when GS-7340 is administered with cobicistat.

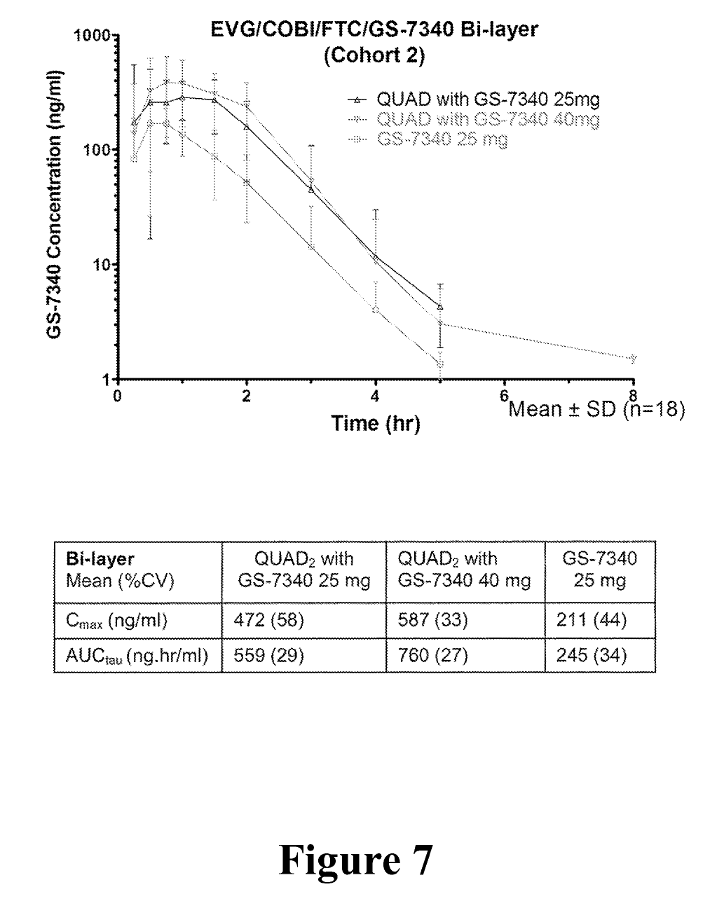

FIG. 7 shows pharmacokinetic data for GS-7340 from patients treated in Cohort 2 (Formulation 2, bilayer). The top line (triangles pointed down) shows GS-7340 concentration (ng/ml) when 40 mg GS-7340 is administered with cobicistat. The middle line (triangles pointed up) shows GS-7340 concentration (ng/ml) when 25 mg GS-7340 is administered with cobicistat. The bottom line (squares) shows GS-7340 concentration (ng/ml) when 25 mg GS-7340 is administered alone. These results also show GS-7340 levels that are 2.2-fold higher for dosing at the 25 mg level when GS-7340 is administered with cobicistat.

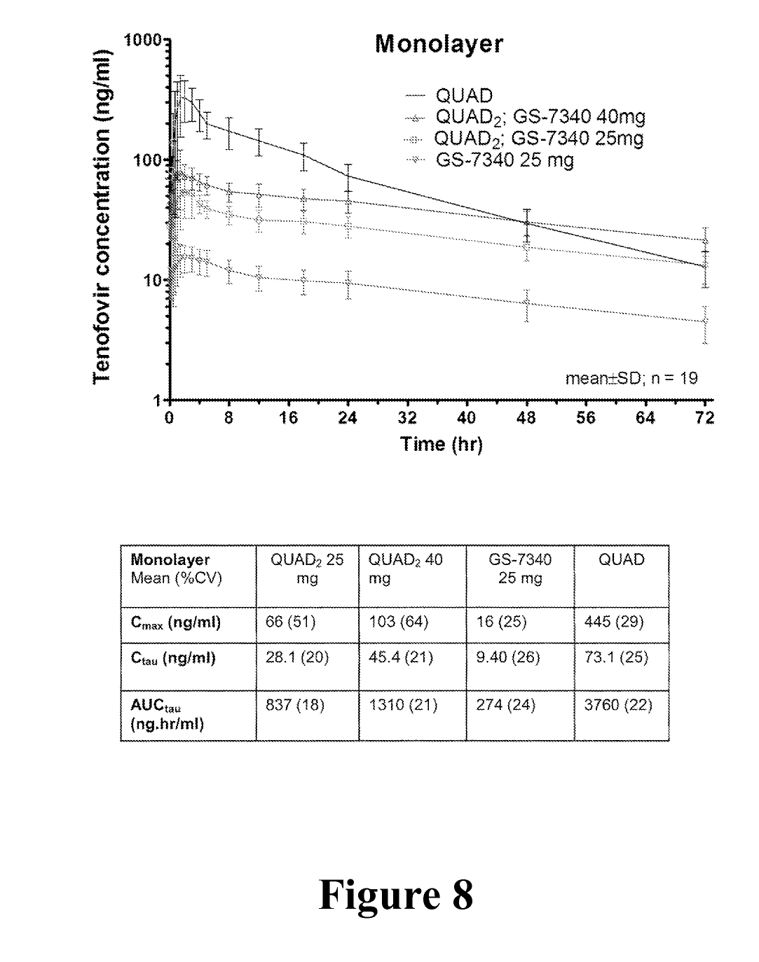

FIG. 8 shows pharmacokinetic data for tenofovir from patients treated in Cohort 1 (Formulation 1, monolayer). The top line (no symbol) shows tenofovir concentration (ng/ml) when 300 mg tenofovir is administered with cobicistat. The next line down (triangles pointed up) shows tenofovir concentration (ng/ml) when 40 mg GS-7340 is administered with cobicistat. The next line down (squares) shows tenofovir concentration (ng/ml) when 25 mg GS-7340 is administered with cobicistat. The bottom line (triangles pointed down) shows tenofovir concentration (ng/ml) when 25 mg GS-7340 is administered alone. These results also show tenofovir levels that are 3-4 fold higher for dosing at the 25 mg level when tenofovir or GS-7340 is administered with cobicistat.

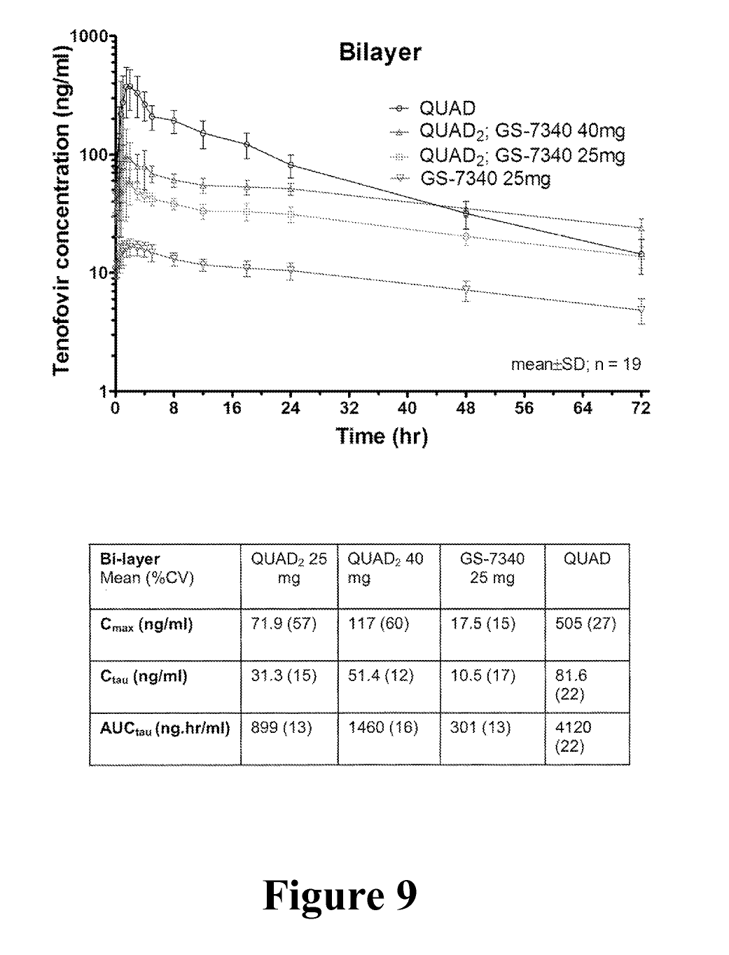

FIG. 9 shows pharmacokinetic data for tenofovir from patients treated in Cohort 2 (Formulation 2, bilayer). The top line (circles) shows tenofovir concentration (ng/ml) when 300 mg tenofovir is administered with cobicistat. The next line down (triangles pointed up) shows tenofovir concentration (ng/ml) when 40 mg GS-7340 is administered with cobicistat. The next line down (squares) shows tenofovir concentration (ng/ml) when 25 mg GS-7340 is administered with cobicistat. The bottom line (triangles pointed down) shows tenofovir concentration (ng/ml) when 25 mg GS-7340 is administered alone. These results also show GS-7340 levels that are 3-4 fold higher for dosing at the 25 mg level when tenofovir or GS-7340 is administered with cobicistat.

Following administration of EVG/COBI/FTC/GS-7340 (25 mg) Formulations 1 and 2, geometric mean GS-7340 and TFV exposures were substantially higher, relative to GS-7340 (25 mg) as a stand-alone agent. With both formulations of EVG/COBI/FTC/GS-7340 (25 mg), GS-7340 AUCₗₐₛₜ and Cₘₐₓ were ˜2.2- and 2.3-fold higher, respectively, whereas TFV AUCₜₐᵤ and Cₘₐₓ were ˜3.1- and 3.7-fold higher, respectively. GS-7340 and TFV exposures were generally dose-proportional following EVG/COBI/FTC/GS-7340 (40 mg) versus EVG/COBI/FTC/GS-7340 (25 mg).

### Biological Example 5

GS-7340 was coformulated with elvitegravir (EVG), cobicistat (COBI), and emtricitabine (FTC) into a single tablet regimen (STR). Across three healthy subject studies, the multiple dose pharmacokinetics (PK) of EVG/COBI/FTC/GS-7340 STR and/or interaction potential between GS-7340 and COBI were evaluated to facilitate GS-7340 dose selection for STR clinical development.

In Study 1 (n=20), subjects received EVG/COBI/FTC/GS-7340 (150/150/200/40 or 150/150/200/25 mg), EVG/COBI/FTC/TDF (150/150/200/300 mg) or GS-7340 25 mg stand alone (SA), 12 days/treatment in a balanced Williams 4×4 design. In Study 2 (n=12), subjects sequentially received GS-7340 (8 mg) SA (Reference) for 12 days and GS-7340 plus COBI (8/150 mg) (Test) for 10 days. In Study 3 (n=34), across two cohorts (each 2×2 crossover design), subjects received EVG/COBI/FTC/GS-7340 (150/150/200/10 mg) (Test, both cohorts), EVG plus COBI (150/150 mg) (Reference, Cohort 1), and FTC plus GS-7340 (200/25 mg) (Reference, Cohort 2), each treatment dosed for 12 days. Statistical comparisons of GS-7340 and TFV were made using geometric mean ratios (GMR), with 90% confidence intervals (CI) of 70-143% (Study 1: Test=EVG/COBI/FTC/GS-7340, Reference=GS-7340 SA). Safety assessments were performed throughout dosing and follow up.

All treatments were generally well tolerated. Study 1 entailed 19/20 completers with one discontinuation from adverse events (AEs) (rhabdomyolysis (Grade 2) while receiving GS-7340 SA). All subjects completed Study 2, while 33 of 34 subjects completed Study 3. No Grade 3 or 4 AE was observed in the studies. In Study 1, when dosed as EVG/COBI/FTC/GS-7340, GS-7340 (25 mg) and resulting TFV exposures were substantially higher versus GS-7340 SA (GMR (90% CI) GS-7340 AUCₗₐₛₜ: 222 (200, 246) and Cₘₐₓ: 223 (187, 265); TFV AUCₜₐᵤ: 307 (290, 324), Cₘₐₓ: 368 (320, 423)). In Study 2, when dosed as GS-7340 plus COBI versus GS-7340 SA, GS-7340 exposures were similarly high, suggesting that the interaction observed in Study 1 was COBI-mediated (GMR (90% CI) GS-7340 AUCₗₐₛₜ: 265 (229, 307) and Cₘₐₓ: 283 (220, 365, TFV AUCₜₐᵤ: 331 (310, 353), Cₘₐₓ: 334 (302, 370), and Cₜₐᵤ: 335 (312, 359)). In Study 3, upon dose adjustment of GS-7340 to 10 mg, EVG/COBI/FTC/GS-7340 (150/150/200/10 mg) versus Reference resulted in comparable GS-7340 and TFV exposures. (GMR (90% CI) GS-7340 AUCₗₐₛₜ: 89.0 (76.7, 103) and Cₘₐₓ: 97.3 (82.1, 115), TFV AUCₗₐₛₜ: 124 (113, 136), Cₘₐₓ: 113 (98.8, 129), and Cₜₐᵤ: 120 (103, 140)). EVG/COBI/FTC/GS-7340 STR provided similar EVG, COBI, and FTC exposures versus reference treatments and historical data.

GS-7340 and TFV exposures increase ˜2-3 fold following coadministration with COBI or as EVG/COBI/FTC/GS-7340 dosing, which may be due to COBI inhibition of Pgp-mediated intestinal secretion of GS-7340. With a 10 mg dose of GS-7340, EVG/COBI/FTC/GS-7340 provided comparable GS-7340 and TFV exposures as GS-7340 at 25 mg and ˜90% lower TFV exposure versus EVG/COBI/FTC/TDF.

### Biological Example 6

EVG/COBI/FTC/TDF and EVG/COBI/FTC/tenofovir alafenamide hemifumarate were administered as single tablet regimens (STR) in a Phase 2 clinical trial evaluating safety and efficacy in HIV+treatment-naïve adults. All subjects had HIV-1 RNA >5000 c/ml. Week 24 data indicated that treatment with the two STRs resulted in 87% of subjects on EVG/COBI/FTC/tenofovir alafenamide hemifumarate and 90% of subjects on EVG/COBI/FTC/TDF having HIV-1 RNA <50 c/ml. The EVG/COBI/FTC/tenofovir alafenamide hemifumarate STR was well tolerated, and relative to the known safety profile of EVG/COBI/FTC/TDF, no new or unexpected adverse drug reactions were identified.

Renal function was assessed in the subjects at week 24. When compared with subjects taking EVG/COBI/FTC/TDF, subjects taking EVG/COBI/FTC/tenofovir alafenamide hemifumarate had significantly less reduction in the estimated glomerular filtration rate (eGFR), a trend towards less proteinuria, and statistically less tubular proteinuria. These differences may represent a reduction in subclinical tenofovir-associated nephrotoxicity.

To assess bone mineral density, dual-energy X-ray absorptiometry scans were performed at baseline and week 24. Subjects taking EVG/COBI/FTC/tenofovir alafenamide hemifumarate experienced a significantly smaller reduction in bone mineral density at both spine and hip after 24 weeks, compared with subjects taking EVG/COBI/FTC/TDF. Importantly, the proportion of subjects with >3% decrease from baseline in hip bone mineral density was 10-fold lower in the EVG/COBI/FTC/tenofovir alafenamide hemifumarate group than the EVG/COBI/FTC/TDF group (3.0% vs. 31.6%).

Together, these data support the hypothesis that TDF-associated renal and bone toxicity is driven by circulating tenofovir, as tenofovir levels are reduced by 90% in subjects administered EVG/COBI/FTC/tenofovir alafenamide hemifumarate.

All references, publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

The use of the terms “a,” “an,” “the,” and similar articles and the like in the context of describing the invention (including the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”), unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention, unless otherwise claimed. No language in the specification should be construed as indicating any nonclaimed element as essential to the practice of the invention.

The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1. A composition comprising: cobicistat, or a pharmaceutically acceptable salt thereof; and tenofovir alafenamide hemifumarate.

2. The composition of claim 1 comprising: 50-500 mg of cobicistat, or a pharmaceutically acceptable salt thereof; and 3-40 mg of tenofovir alafenamide hemifumarate.

3. The composition of claim 1, further comprising a pharmaceutically acceptable carrier or diluent.

4. A method of treating a viral infection in a human comprising administering a composition of claim 1 to the human.

5. A method of treating a viral infection in a human comprising coadministering cobicistat, or a pharmaceutically acceptable salt thereof, and tenofovir alafenamide hemifumarate, to the human.

6. A method of inhibiting activity of a retroviral reverse transcriptase comprising coadministering cobicistat, or a pharmaceutically acceptable salt thereof, and tenofovir alafenamide hemifumarate.

7. The method of claim 6, wherein the coadministering of cobicistat, or a pharmaceutically acceptable salt thereof, and tenofovir alafenamide hemifumarate, is in a human.

8-11. (canceled)

12. A method of boosting an anti-viral effect of tenofovir alafenamide hemifumarate in a human comprising administering a composition of claim 1 to the human.

13. A method of boosting an anti-viral effect of tenofovir alafenamide hemifumarate in a human comprising coadministering cobicistat, or a pharmaceutically acceptable salt thereof, and tenofovir alafenamide hemifumarate to the human.

14. The method of claim 13, wherein 50-500 mg of cobicistat, or a pharmaceutically acceptable salt thereof, is coadministered with 3-40 mg of tenofovir alafenamide hemifumarate.

15. A method of inhibiting Pgp-mediated intestinal secretion of tenofovir alafenamide hemifumarate in a human comprising administering a composition of claim 1 to the human.

16. A method of inhibiting Pgp-mediated intestinal secretion of tenofovir alafenamide hemifumarate in a human by coadministration of cobicistat, or a pharmaceutically acceptable salt thereof, and tenofovir alafenamide hemifumarate.

17. The method of claim 16, wherein 50-500 mg of cobicistat, or a pharmaceutically acceptable salt thereof, is coadministered with 3-40 mg of tenofovir alafenamide hemifumarate.

18. The method of claim 5, wherein the viral infection is human immunodeficiency virus (HIV) or Hepatitis B virus (HBV).

19. (canceled)

20. The method of claim 13, wherein the virus is human immunodeficiency virus (HIV) or Hepatitis B virus (HBV).

21. A composition comprising: (a) tenofovir alafenamide hemifumarate; (b) cobicistat, or a pharmaceutically acceptable salt thereof; (c) emtricitabine; and (d) elvitegravir.

22. A composition comprising: (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir.

23. A method of treating a viral infection in a human comprising administering a composition of claim 21 to the human.

24. A method of treating a viral infection in a human comprising coadministering (a) tenofovir alafenamide hemifumarate; (b) cobicistat, or a pharmaceutically acceptable salt thereof; (c) emtricitabine; and (d) elvitegravir to the human.

25. The method of claim 24 comprising coadministering (a) 3-40 mg tenofovir alafenamide hemifumarate; (b) 50-500 mg cobicistat, or a pharmaceutically acceptable salt thereof; (c) 50-500 mg emtricitabine; and (d) 50-500 mg elvitegravir to the human.

26-30. (canceled)

31. The method of claim 24, wherein the viral infection is human immunodeficiency virus (HIV) or Hepatitis B virus (HBV).

32. (canceled)
